(19) United States
(12) Patent Application Publication
(10) Pub. No.: US 2026/0355471 A1
(43) Pub. Date: Nov. 12, 2026

(54) METHODS FOR THE ENDOVASCULAR TREATMENT OF HYDROCEPHALUS AND ELEVATED INTRACRANIAL PRESSURE

(71) Applicant: CEREVASC, INC., Charlestown, MA (US)

(72) Inventors: Adel M. Malek, Charlestown, MA (US); Stephen Conroy, Charlestown, MA (US); Travis Carbonneau, Charlestown, MA (US)

(73) Assignee: CEREVASC, INC., Charlestown, MA (US)

(21) Appl. No.: 19/673,986

(22) Filed: May 11, 2026

Related U.S. Application Data
(60) Provisional application No. 63/804,314, filed on May 12, 2025.

(51) Int. Cl. A61M 25/06; A61B 17/00; A61B 17/34; A61B 34/20; A61B 90/00; A61M 25/00; A61M 27/00

(52) U.S. Cl. CPC A61M 25/0662; A61B 17/00234; A61B 17/3403; A61B 17/3417; A61B 34/20; A61B 90/37; A61M 25/0021; A61M 27/002; A61B 2017/00238; A61B 2017/00305; A61B 2017/3454; A61B 2034/2046; A61B 2090/376; A61M 2025/0042; A61M 2025/0681; A61M 2210/0687; A61M 2210/12

(57) ABSTRACT

Methods for endovascularly deploying a shunt in a patient are provided. The methods comprise introducing a delivery catheter and a microcatheter in a patient. The method further comprises navigating the delivery catheter and microcatheter through a jugular vein, into a venous sinus of the patient, removing the microcatheter from the lumen of the delivery catheter, navigating a distal portion of the delivery catheter to a target penetration site within the venous sinus of the patient, introducing a shunt into the lumen of the delivery catheter, advancing the shunt through the lumen of the delivery catheter within the venous sinus of the patient, distally advancing a penetrating element coupled to a distal portion of the delivery catheter through a wall in the venous sinus at the target penetration site to create an anastomosis between an SAS and the venous sinus, and implanting the shunt from the delivery catheter into the anastomosis.

### RELATED APPLICATIONS

The present application claims the benefit of priority under 35 U.S.C. §119 to U.S. Provisional Application Ser. No. 63/804,314, filed May 12, 2025.

### FIELD OF THE INVENTION

The inventions disclosed herein relate to improved methods for accessing subarachnoid spaces, and for endovascularly deploying a shunt to drain cerebrospinal fluid (CSF), (e.g., to relieve elevated intracranial pressure or treat normal pressure hydrocephalus). More particularly, the present disclosure pertains to methods for treatment of hydrocephalus, pseudotumor cerebri, and/or intracranial hypertension.

### BACKGROUND

There has been significant progress made in recent decades in the development and practice of minimally invasive surgical devices and procedures. As used herein, “minimally invasive” refers to the use of surgical devices and implants that access the body via the vasculature via arterial or venous access in the groin, arm or neck area, as opposed to more invasive traditional procedures that access the body more directly through percutaneous/solid tissue incisions and cutting and/or boring through bones, as needed to access the internal area of the body on which the procedure is performed. Minimally invasive may also refer to the to the use of surgical devices and implants that access the body via other natural body orifices, cavities and tubular structures, such as the esophagus, intestines, bronchial passageways, etc. Percutaneous access through the skin into solid tissue, e.g., for accessing the liver, prostate or lungs, e.g., using a trocar and stylet, may also be considered minimally invasive.

Examples of minimally invasive surgical devices and procedures to relieve elevated intracranial pressure or treat normal pressure hydrocephalus are described in United States Patents Nos. 10,272,230 and 10,765,846 and United States Patent Application Publication No. US20200030588A1, the entire disclosures of which are incorporated herein by reference, as though set forth in full.

There is an ongoing need, however, to improve minimally invasive neurosurgical devices that can access the intracranial subarachnoid space (“SAS”) and deploy devices designed to drain cerebrospinal fluid from the SAS to the venous system. The complexity of the device preparation steps, impact of the preparation steps to the devices, and time required to prepare the minimally invasive system for clinical use are important considerations, and it is desirable to simplify and complete such preparation in less than an hour, and preferably less than 15 minutes. Improvements to enhance the user experience by simplifying the device deployment procedure and mitigating risks to patient safety during the minimally invasive procedure are also needed. Further, improvements are needed to enhance the manufacturability of the minimally invasive neurosurgical devices such that the devices can be manufactured at reduced costs and at larger volumes to enable commercialization of the technology.

One minimally invasive surgical technique, referred to as an endovascular CSF shunt delivery procedure, involves treating hydrocephalus, pseudotumor cerebri, and/or intracranial hypertension, which is caused by excess cerebrospinal fluid (CSF) fluid in the SAS of the patient—a naturally occurring separation between the pia mater and the arachnoid layer in the cranium of the patient where CSF pools. Typically, such CSF is passed into the SAS over the cerebral hemispheres and then into the venous system by arachnoid granulations, where the CSF is reabsorbed into the body of the patient. The endovascular CSF shunt delivery procedure involves endovascularly creating an anastomosis between the lumen of the inferior petrosal sinus (IPS) (or other dural venous sinus) and the SAS of the patient, implanting an endovascular shunt device within the anastomosis, and draining the excess CSF from the SAS into the venous system of the patient via the implanted shunt device. Examples of endovascular devices and procedures to drain CSF from the CSF-filled SAS into the venous system of a patient are described in U.S. Patent Nos. 10,272,230 and 10,765,846 and U.S. Patent Publication Nos. 2020/0030588 and 2024/0390643, the entire disclosures of which are expressly incorporated herein by reference as though set forth in full.

Conventional techniques for establishing endovascular access between a venous sinus, such as the IPS, and the SAS often utilize a distal anchoring construct to facilitate navigation and positioning of a delivery system within the venous anatomy. In such approaches, an anchor is first introduced through a microcatheter and deployed distal to the intended penetration site within the IPS. A stent or anchoring element may then be deployed on the anchor to provide a distal reference and support structure to assist advancement of a delivery catheter to the target site. The microcatheter is subsequently exchanged for a delivery catheter and shunt delivery system, which is advanced to the target IPS location for creation of the anastomosis and deployment of the shunt into the SAS. In conventional techniques, a needle associated with the distal end of the delivery catheter or a stylet advanced from a lumen of the delivery catheter is used to create the anastomosis between the IPS and the SAS. Following deployment, additional device exchanges may be performed, including reintroduction of a microcatheter and removal of the delivery catheter, and eventual retrieval of the distal anchor via the microcatheter.

This multi-step sequence requires multiple device exchanges across delicate neurovascular anatomy, increasing procedural complexity, fluoroscopy time, and the potential for catheter instability during transitions. Shorter procedure time can also decrease the total time that patients require general anesthesia, which may further mitigate patient risk, particularly in patients over 60 years of age and/or those with comorbidities that increase the risks associated with general anesthesia. Additionally, the presence of an expanded anchoring or stent structure distal to the target site may introduce radial forces or mechanical interaction with adjacent neuroanatomical structures, including cranial nerves, which may contribute to irritation or adverse neurological effects.

Accordingly, there remains a need for simplified delivery systems that reduce overall procedure time and device exchanges, and eliminate reliance on distal anchoring constructs for navigation, while maintaining precise control of access and deployment at the IPS–SAS interface.

Access to the SAS via the IPS requires precise navigation of delivery catheters, microcatheters, guide wires, and/or needles through complex venous anatomy. It is critical to ensure that the needle that traverses the wall of the IPS to create the anastomosis be appropriately positioned within the SAS to ensure that the resulting anastomosis is sufficient for subsequent deployment of the shunt into the SAS. In addition, it is important that penetration of the IPS wall occurs at a correct and intended location along the IPS to avoid entry at anatomically unfavorable or unsafe points that may increase the risk of misdirection into adjacent arterial, venous, or critical central nervous system structures or reduce the likelihood of successful downstream delivery. Inadequate penetration, incorrect entry location, or misinterpretation of needle position may result in partial engagement of the IPS wall or incorrect termination within adjacent venous structures, thereby compromising subsequent deployment of the shunt.

Significantly, the IPS follows an obliquely oriented, three-dimensional course that is not aligned with standard fluoroscopic imaging planes. In particular, in conventional biplane angiographic techniques, the frontal (anteroposterior) projection and orthogonal lateral projection are commonly used to visualize neurovascular structures. However, when applied to the IPS, these standard projections are suboptimal because they do not accurately represent the true longitudinal course of the segment of the IPS that is targeted for penetration into the SAS. As a result, the targeted IPS segment may appear foreshortened in conventional frontal and lateral projections, leading to an inaccurate representation of its apparent length and obscuring the relationship between the true anatomical course of the targeted IPS segment and the apparent position of a catheter or needle within it. Accordingly, the extent of device advancement within the targeted IPS segment as shown in conventional projections may not reliably correspond to its actual intravascular position, and precise determination of a penetration location along the targeted IPS segment may be limited. In addition, conventional frontal and lateral projections do not provide a coordinated set of complementary views that enable accurate assessment of needle trajectory relative to the IPS wall. For example, the lateral projection may suffer from superimposition of bilateral structures and limited ability to distinguish ipsilateral from contralateral anatomy, while the frontal projection alone does not provide sufficient orthogonal perspective to localize the axial position of a needle exit point. As a result, conventional imaging does not reliably permit confirmation of whether the needle has fully traversed the IPS wall or whether the distal portion of the needle has entered and advanced sufficiently within the SAS.

Accordingly, there is a need for imaging projection techniques that reduce foreshortening of the IPS, improve visualization of its longitudinal course, and enable more accurate assessment of delivery catheter and needle position, IPS wall penetration location, and confirmation of traversal into the SAS from the IPS.

### SUMMARY

In accordance with a first aspect of the present inventions, a method for deploying a shunt having a lumen and a valve in a patient is provided.

The method comprises introducing a delivery catheter and one or more elongate navigation members (e.g., a microcatheter and a guide wire disposed within a lumen of the microcatheter) into a venous access point in the patient. The elongate navigation member(s) are disposed within a lumen of the delivery catheter. The method comprises navigating the delivery catheter and elongate navigation member(s) through a jugular vein and into a venous sinus of the patient and removing the elongate navigation member(s) from the lumen of the delivery catheter. The method further comprises distally advancing a distal portion of the delivery catheter distal to a targeted segment of the venous sinus.

The method further comprises introducing the shunt into the lumen of the delivery catheter. In one method, introducing the shunt into the lumen of the delivery catheter may comprise connecting a transfer device to a handle of delivery catheter, and disposing a shroud assembly within a lumen of the transfer device. The shroud assembly may comprise a shroud having a lumen in which the shunt is disposed and a delivery wire connected to the shroud. This method may further comprise distally advancing the shroud assembly, while the shunt is disposed within the lumen of the shroud, from the lumen of the transfer device into the lumen of the delivery catheter. This method may further comprise flushing a lumen of the shunt and performing a valve patency confirmation test on the shunt before distally advancing the shunt assembly from the lumen of the transfer device into the lumen of the delivery catheter.

The method further comprises distally advancing a shunt through the lumen of the delivery catheter within the venous sinus of the patient, exposing a penetrating element coupled to a distal portion of the delivery catheter when the distal portion of the delivery catheter is distal to the targeted segment of the venous sinus, and proximally withdrawing the distal portion of the delivery catheter to the targeted segment of the venous sinus subsequent to exposing the penetrating element.

The method further comprises distally advancing the exposed penetrating element through a wall in the venous sinus at the targeted segment of the venous sinus to create an anastomosis between an intracranial subarachnoid space (SAS) and the venous sinus, and implanting the shunt from the delivery catheter into the anastomosis. In one method, the venous sinus is an inferior petrosal sinus (IPS), in which case, the anastomosis may be created between the IPS and a cerebellopontine (CP) angle cistern of the SAS. In one method, the shunt comprises a self-expanding distal anchoring mechanism, in which case, implanting the shunt from the delivery catheter into the anastomosis may comprise deploying the distal anchoring mechanism in the SAS. An optional method may further comprise, after deploying the distal anchoring mechanism in the SAS, applying a proximal force to the delivery catheter while the shunt remains engaged with a proximal retention feature of a shroud disposed within the lumen of the delivery catheter, and assessing sufficient deployment of the distal anchoring mechanism in the SAS based on a resistance to the proximal force applied to the delivery catheter. If a shroud assembly is used, it may comprise a shroud retention feature that retains a proximal portion of the shunt when the shroud assembly is disposed within the lumen of the delivery catheter, in which case, the method may further comprise unsheathing the shroud assembly from the lumen of the delivery catheter lumen, thereby releasing the proximal portion of the shunt from the retention feature.

In accordance with a second aspect of the present inventions, a method for fluoroscopic visualization during a medical procedure that accesses a subarachnoid space (SAS) from the inferior petrosal sinus (IPS) of a patient is provided.

The method comprises configuring a fluoroscopic imaging system to acquire images of the IPS during the medical procedure. The method comprises acquiring a first fluoroscopic image of the IPS in a first oblique projection including a rotational angulation in the range of 25°-35° relative to an anteroposterior (AP) reference projection and a cranial angulation relative to a cranial-caudal axis of the patient in the range of 25°-35°. As one example, the first oblique projection may comprise a right anterior (RAO)/cranial projection if the IPS is a right IPS and a left anterior (LAO)/cranial projection if the IPS is a left IPS. The method further comprises acquiring a second fluoroscopic image of the IPS in a second oblique projection different from the first oblique projection to provide a complementary view of the IPS. In one method, the second oblique projection may include a rotational angulation in the range of 25°-35° relative to a lateral or transverse axis and a cranial or caudal angulation relative to the cranial-caudal axis of the patient in the range of 25°-35°. In another method, the first and second oblique projections are orthogonal to each other.

In one method, the fluoroscopic imaging system is a single-plane fluoroscopic imaging system, in which case, the first and second fluoroscopic images are sequentially acquired. In another method, the fluoroscopic imaging system may be a bi-plane fluoroscopic imaging system, in which case, the first and second fluoroscopic images may be simultaneously acquired. For example, the biplane fluoroscopic imaging system may comprise a frontal imaging arm and a lateral imaging arm, in which case, the frontal imaging arm may acquire the first fluoroscopic image and the lateral imaging arm may acquire the second fluoroscopic image.

The method further comprises visualizing the first and second fluoroscopic image while performing the medical procedure. In one method, visualizing the first and second fluoroscopic images while performing the medical procedure may comprise guided advancement of a delivery catheter and a penetrating element associated with the delivery catheter. The guided advancement of the delivery catheter and penetrating element may comprise determining a location of a penetration of a wall of the IPS along a longitudinal course of the IPS based on the first fluoroscopic image and/or assessing whether the penetrating element has traversed a wall of the IPS and entered the SAS based on the first and second fluoroscopic images.

In accordance with a third aspect of the present inventions, a method for fluoroscopic visualization during a medical procedure that accesses a subarachnoid space (SAS) from an inferior petrosal sinus (IPS) of a patient is provided.

The method comprises configuring a fluoroscopic imaging system to acquire images of the IPS during the medical procedure. The method further comprises acquiring a first fluoroscopic image of the IPS in a first oblique projection configured for reducing foreshortening of a targeted segment of the IPS relative to a conventional anteroposterior (AP) projection. As one example, the first oblique projection may comprise a right anterior (RAO)/cranial projection if the IPS is a right IPS and a left anterior (LAO)/cranial projection if the IPS is a left IPS. As another example, the first oblique projection includes a rotational angulation in the range of 25°-35° relative to an anteroposterior (AP) reference projection and a cranial angulation relative to a cranial-caudal axis of the patient in the range of 25°-35°.

The method further comprises acquiring a second fluoroscopic image of the IPS in a second oblique projection different from the first oblique projection and configured to provide a complementary, at least partially orthogonal, view of the targeted segment of the IPS. In one method, the second oblique projection may include a rotational angulation in the range of 25°-35° relative to a lateral or transverse axis and a cranial or caudal angulation relative to the cranial-caudal axis of the patient in the range of 25°-35°. In another method, the first and second oblique projections are orthogonal to each other.

The method further comprises visualizing the first and second fluoroscopic image while performing the medical procedure. For example, the method may comprise collectively using the first fluoroscopic image and the second fluoroscopic image to improve visualization of a longitudinal course of the IPS and a spatial relationship of the delivery catheter and penetrating element relative to the IPS and SAS. In one method, the first oblique projection enhances visualization of a delivery catheter and a penetrating element associated with the delivery catheter within a longitudinal course of the IPS and facilitating assessment of a trajectory of the penetrating element along the targeted segment of the IPS, while the second oblique projection facilitates confirmation that the penetrating element has traversed a wall of the IPS and identification of an exit location of the penetrating element in the targeted segment of the IPS.

In another method, the fluoroscopic imaging system is a single-plane fluoroscopic imaging system, in which case, the first and second fluoroscopic images are sequentially acquired. In another method, the fluoroscopic imaging system may be a bi-plane fluoroscopic imaging system, in which case, the first and second fluoroscopic images may be simultaneously acquired. For example, the biplane fluoroscopic imaging system may comprise a frontal imaging arm and a lateral imaging arm, in which case, the frontal imaging arm may acquire the first fluoroscopic image and the lateral imaging arm may acquire the second fluoroscopic image.

In accordance with a fourth aspect of the present inventions, a method for deploying a shunt comprising a lumen and a valve in a patient is provided.

The method comprises introducing a delivery catheter and one or more elongate navigation members (e.g., a microcatheter and a guide wire disposed within a lumen of the microcatheter) into a venous access point in the patient. The elongate navigation member(s) are disposed within a lumen of the delivery catheter. The method further comprises navigating the delivery catheter and one or more elongate navigation members through a jugular vein and into a venous sinus of the patient, navigating a distal portion of the delivery catheter within a targeted segment of the venous sinus of the patient, and removing the elongate navigation member(s) from the lumen of the delivery catheter.

The method further comprises introducing the shunt into the lumen of the delivery catheter. In one method, introducing the shunt into the lumen of the delivery catheter may comprise connecting a transfer device to a handle of delivery catheter, and disposing a shroud assembly within a lumen of the transfer device. The should assembly may comprise a shroud having a lumen in which the shunt is disposed and a delivery wire connected to the shroud. This method may further comprise distally advancing the shroud assembly, while the shunt is disposed within the lumen of the shroud, from the lumen of the transfer device into the lumen of the delivery catheter. This method may further comprise flushing a lumen of the shunt and performing a valve patency confirmation test on the shunt before distally advancing the shunt assembly from the lumen of the transfer device into the lumen of the delivery catheter.

The method further comprises distally advancing a shunt through the lumen of the delivery catheter within the venous sinus of the patient, distally advancing a penetrating element coupled to a distal portion of the delivery catheter through a wall in the venous sinus at the targeted segment of the venous sinus to create an anastomosis between an intracranial subarachnoid space (SAS) and the venous sinus, and implanting the shunt from the delivery catheter into the anastomosis.

In one method, the venous sinus is an inferior petrosal sinus (IPS), in which case, the anastomosis may be created between the IPS and a cerebellopontine (CP) angle cistern of the SAS. In one method, the shunt comprises a self-expanding distal anchoring mechanism, in which case, implanting the shunt from the delivery catheter into the anastomosis may comprise deploying the distal anchoring mechanism in the SAS. An optional method may further comprise, after deploying the distal anchoring mechanism in the SAS, applying a proximal force to the delivery catheter while the shunt remains engaged with a proximal retention feature of a shroud disposed within the lumen of the delivery catheter, and assessing sufficient deployment of the distal anchoring mechanism in the SAS based on a resistance to the proximal force applied to the delivery catheter. If a shroud assembly is used, it may comprise a shroud retention feature that retains a proximal portion of the shunt when the shroud assembly is disposed within the lumen of the delivery catheter, in which case, the method may further comprise unsheathing the shroud assembly from the lumen of the delivery catheter lumen, thereby releasing the proximal portion of the shunt from the retention feature.

Other and further aspects and features of embodiments will become apparent from the ensuing detailed description in view of the accompanying figures.

### BRIEF DESCRIPTION OF THE DRAWINGS

FIG. 1 is a plan view of one embodiment of a delivery assembly constructed in accordance with the present inventions;

FIGS. 2A-2D are perspective, top and side views of a delivery catheter of the delivery assembly of FIG. 1;

FIGS. 3A- 3F are side, perspective and cross-sectional views of the distal portion of the delivery catheter of FIGS. 2A-2D;

FIGS. 4A-4B are side views illustrating an interaction between a penetrating and guard member of the distal portion of the delivery catheter of FIGS. 2A-2D;

FIGS. 5A-5C are axial views of the distal portion of the delivery catheter of FIGS. 2A-2D;

FIGS. 6A-6N are side and perspective views of different embodiments of penetrating elements of the delivery catheter of FIGS. 2A-2D;

FIGS. 7A-7D are side views of one embodiment of an assembly of a shunt and a shroud that can be delivered into the vasculature of a patient by the delivery assembly of FIG. 1;

FIGS. 8A-8 F are side and detailed views illustrating the interaction between the shunt and shroud assembly of FIGS. 7A-7D and the delivery catheter of FIGS. 2A-2D;

FIGS. 9A-9E are perspective and cross-sectional views of the shroud of FIGS. 8A-8F;

FIGS. 10A-10E are perspective, detailed and cross-sectional views of the distal end of the shunt of FIGS. 7A-7D;

FIGS. 11A-11C are perspective and axial views of one embodiment of an anchoring mechanism, a proximal radiopaque marker, and an elongate guide member of the shunt of FIGS. 7A-7D;

FIGS. 12A-12B are perspective and axial views of the proximal radiopaque marker of FIGS. 11A-11C;

FIG. 13 is a cross-sectional view of one embodiment of a handle of the delivery catheter of FIGS. 2A-2D;

FIGS. 14A-14D are perspective and cross-sectional views of an alternative embodiment of a handle the delivery catheter of FIGS. 2A-2D;

FIGS. 15A-15G are perspective, cross-sectional and detailed views of one embodiment of a transfer device that can be used to transfer the shunt and shroud assembly of FIGS. 7A-7D and the delivery catheter of FIGS. 2A-2D into a guide catheter of the delivery assembly of FIG. 1;

FIGS. 16A-16B is a perspective view of the transfer device of FIGS. 15A-15F coupled to the delivery catheter of FIGS. 2A-2D;

FIGS. 17A-17B are perspective and top views of one embodiment of a reservoir assembly interacting with the transfer device of FIGS. 15A-15C and 16A-16B;

FIG. 18 is a flow chart of one method of performing a patency test on the shunt of FIGS. 7A-7D using the reservoir assembly of FIGS. 17A-17B and the transfer device of FIGS. 15A-15D and 16A-16B;

FIG. 19 is a plan view of another embodiment of a delivery assembly constructed in accordance with the present inventions;

FIG. 20 is an anterior view of a head of a human patient, particularly showing relevant vascular structures of the head;

FIG. 21 is a median sagittal view of a head of the human patient, particularly showing relevant vascular structures of the head;

FIG. 22 is a flow chart of an endovascular shunt deployment procedure using the other delivery assembly of FIG. 19, according to embodiments of the disclosed inventions;

FIGS. 23A-23F are plan views showing the use of the endovascular shunt deployment procedure for deploying the shunt of FIGS. 7A-7D within the anatomy of patient;

FIGS. 24A-24F are plan views of actual fluoroscopic images corresponding to the use of the endovascular shunt deployment procedure for deploying the shunt of FIGS. 7A-7D within in vitro anatomic model;

FIG. 25 is perspective view of one embodiment of a bi-plane fluoroscopic imaging system that can be used to image a patient during an endovascular shunt deployment procedure;

FIGS. 26A-26B are schematics of two orthogonal image planes that can be used to fluoroscopically image a patient;

FIGS. 27A-27B are schematics illustrating imaging of an anatomy at the base of a skull of the patient in a conventional anterior-posterior (AP) and lateral projections, particularly showing a delivery catheter and associated penetrating element within a targeted inferior petrosal sinus (IPS) segment of the patient;

FIGS. 28A-28B are schematics illustrating imaging of an anatomy at the base of a skull of a patient in an AP oblique projection and lateral oblique projection performed in accordance with the present inventions, particularly showing a delivery catheter and associated penetrating element within a targeted IPS of the patient;

FIGS. 29A-29B are schematics illustrating an AP right anterior oblique (RAO)-cranial projection and a lateral RAO-cranial projection of an acquired three-dimensional angiogram of an anatomy at the base of a skull of a patient;

FIGS. 30A-30B are schematics illustrating an AP left anterior oblique (LAO)-cranial projection and a lateral LAO-caudal projection of an acquired three-dimensional angiogram of an anatomy at the base of a skull of a patient; and

FIGS. 31A-31F are different imaging views of a skull of a patient for creating the projections of FIGS. 27A-27B, FIGS. 29A- 29B, and FIGS. 30A- 30B.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skilled in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Various embodiments are described hereinafter with reference to the figures. The figures are not necessarily drawn to scale, the relative scale of select elements may have been exaggerated for clarity, and elements of similar structures or functions are represented by like reference numerals throughout the figures. It should also be understood that the figures are only intended to facilitate the description of the embodiments, and are not intended as an exhaustive description of the invention or as a limitation on the scope of the invention, which is defined only by the appended claims and their equivalents. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

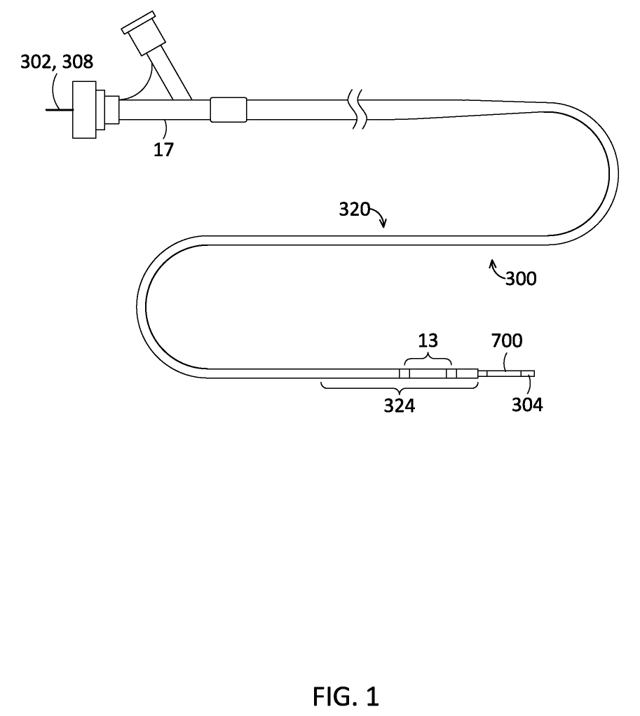

FIG. 1 is a side view of a delivery assembly 300 for accessing the subarachnoid space and/or delivering an endovascular cerebrospinal fluid (CSF) implant (e.g., shunt, implant/shunt) into a target site of a patient, in accordance with embodiments of the disclosed inventions. The delivery assembly 300 may include an anchor 700 (not shown) and the shunt (not shown) detachably coupled to the delivery assembly 300. The delivery assembly 300 and the shunt may be composed of suitable biocompatible materials. The delivery assembly 300 is dimensioned to reach remote locations of the vasculature and is configured to deliver the shunt percutaneously to the target location (e.g., cerebellopontine angle cistern (CP angle cistern), inferior petrosal sinus (IPS)). The delivery assembly 300 includes a tubular member interface having an outer tubular member 320 (i.e., guide catheter) and an inner tubular member 304 (i.e., delivery catheter / micro catheter) coaxially disposed within the outer tubular member 320 and movable relative to the outer tubular member 320. The delivery assembly 300 may include a guidewire 302 coaxially disposed within the guide catheter 320 and/or the delivery catheter 304. The guidewire 302 can be, for example, 0.035’’ (0.889 mm) in diameter. Additionally to the guidewire 302, the delivery assembly 300 may include a delivery wire 308 disposed within the delivery catheter 304. The delivery wire 308 has a smaller diameter (e.g., approximately 0.010’’ (0.254 mm) to 0.018’’ (0.4572 mm) or other suitable dimension to facilitate accessing intracranial venous vasculature with other components of delivery assembly 300) compared to guidewire 302.

The guide catheter 320, delivery catheter 304, and guidewires 302/308 (FIG. 1) may be formed of suitable biocompatible materials, and may include markings 13 for purposes of imaging (e.g., markers composed of radio-opaque materials). Various known and often necessary accessories to the delivery assembly 300, e.g., one or more radiopaque marker bands 13 at the distal portion 324 of the guide catheter 320 to allow viewing of the position of the distal portion under fluoroscopy and a Luer assembly 17 for guidewires and/or fluids access, are shown in FIG. 1. The delivery assembly 300 and/or the shunt may include a penetrating element (not shown) configured to pierce and/or penetrate a venous sinus wall (e.g., IPS wall and arachnoid layer to access a CP angle cistern). The delivery catheter 304, with or without a delivery guide wire, facilitates navigation and delivery of the shunt through the patient’s vasculature to a target deployment location.

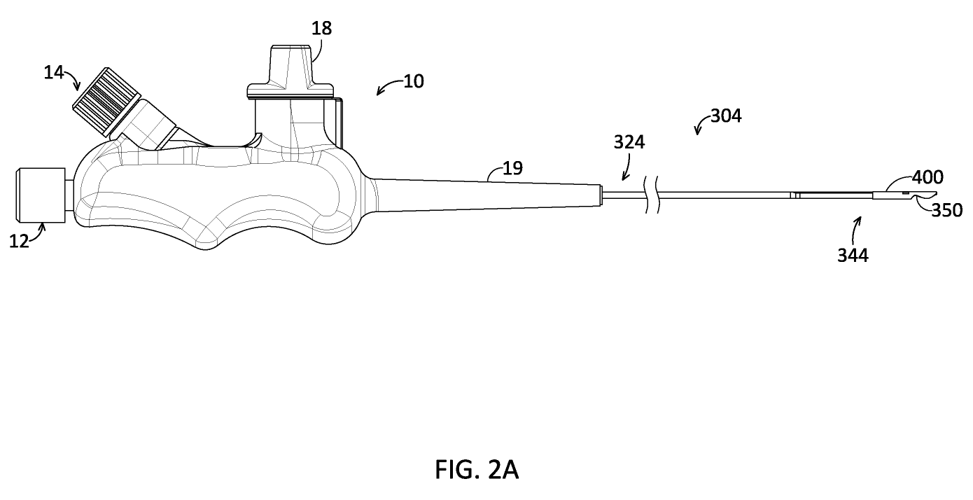

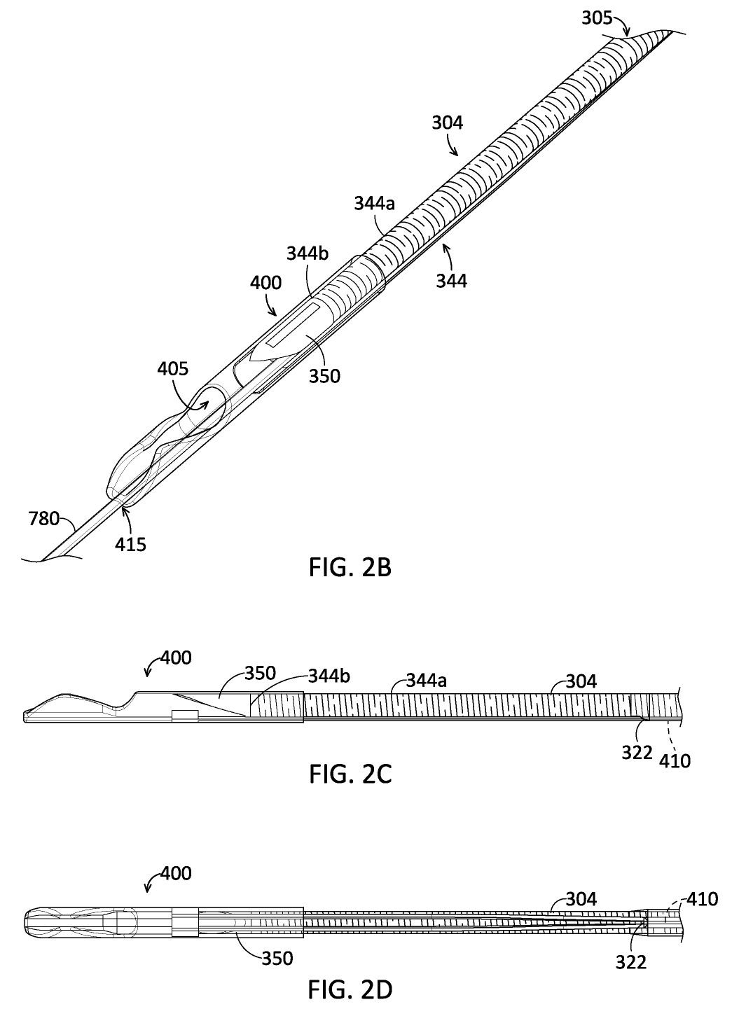

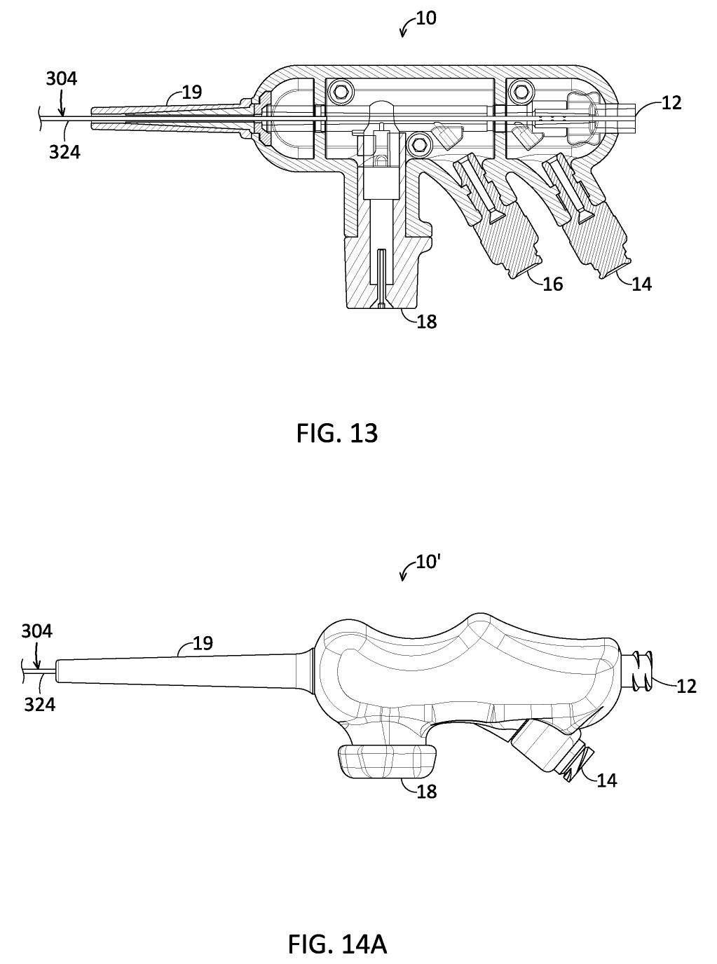

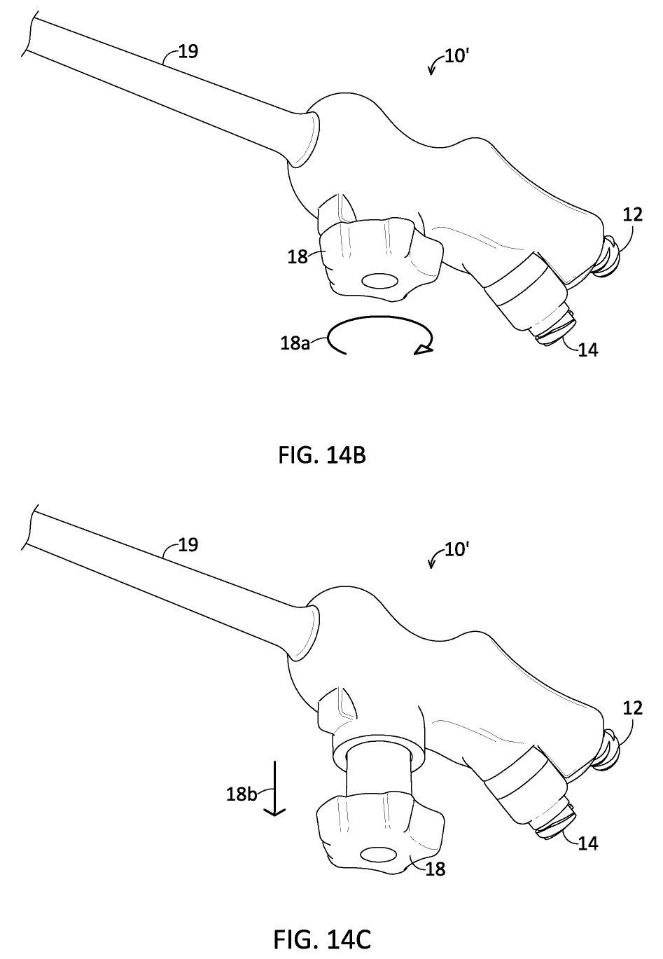

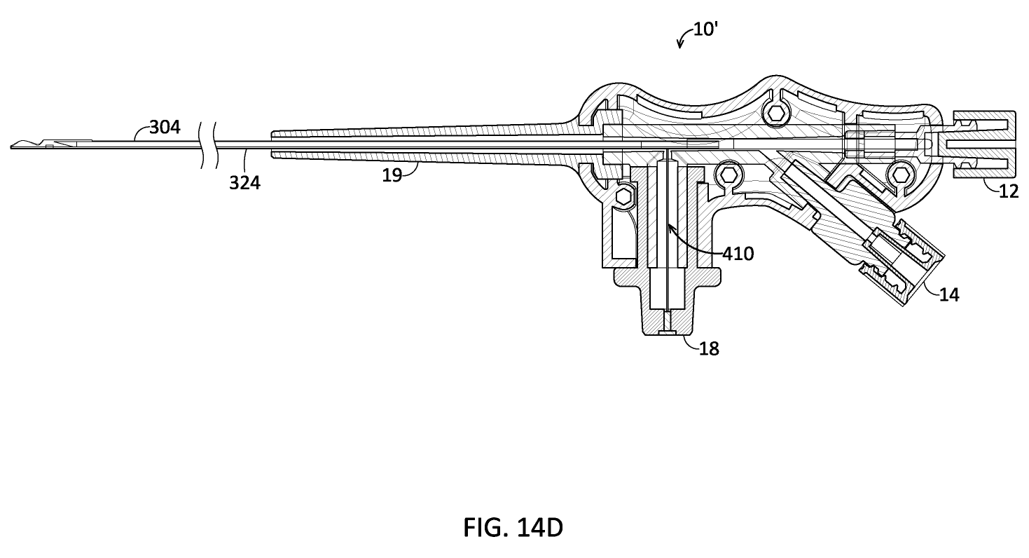

FIG. 2A illustrates a side view of the delivery catheter 304 according to another embodiment of the inventions. Instead of the Luer assembly 17 of FIG. 1, the delivery catheter 304 is coupled and/or integrated to a handle 10, as shown in FIG. 2A. The handle 10 comprises a Luer fitting 12, a delivery lumen flush port 14, a tether retraction mechanism 18, and a strain relief 19; which will be further described in FIGS. 13-14C below. The delivery catheter 304 comprises a proximal portion 324; the proximal portion 324 may be coupled and/or integrated to the handle 10 through the strain relief 19. The delivery catheter 304 further comprises a distal portion 344 (detailed view of FIG. 2B); the distal portion 344 comprises a penetrating element 350 disposed within a guard member 400, which will be further described in FIGS. 2B-D. Embodiments of delivery catheter 304 can include a multi-layer construction. For example, the delivery catheter 304 can be manufactured by having a reinforcing member, such as a hypotube with regions of different flexibility (e.g., interrupted spiral cut, selective slots, fenestrations or their like), where the reinforcing member interior surface is coated, dipped or covered by a lubricious material, such as PTFE lining, and the reinforcing member exterior surface is coated, dipped or covered by a polymeric jacket, as described in U.S. Patent Nos. 10,272,230 and 10,765,846 and U.S. Patent App. Pub. No. US20200030588A1.

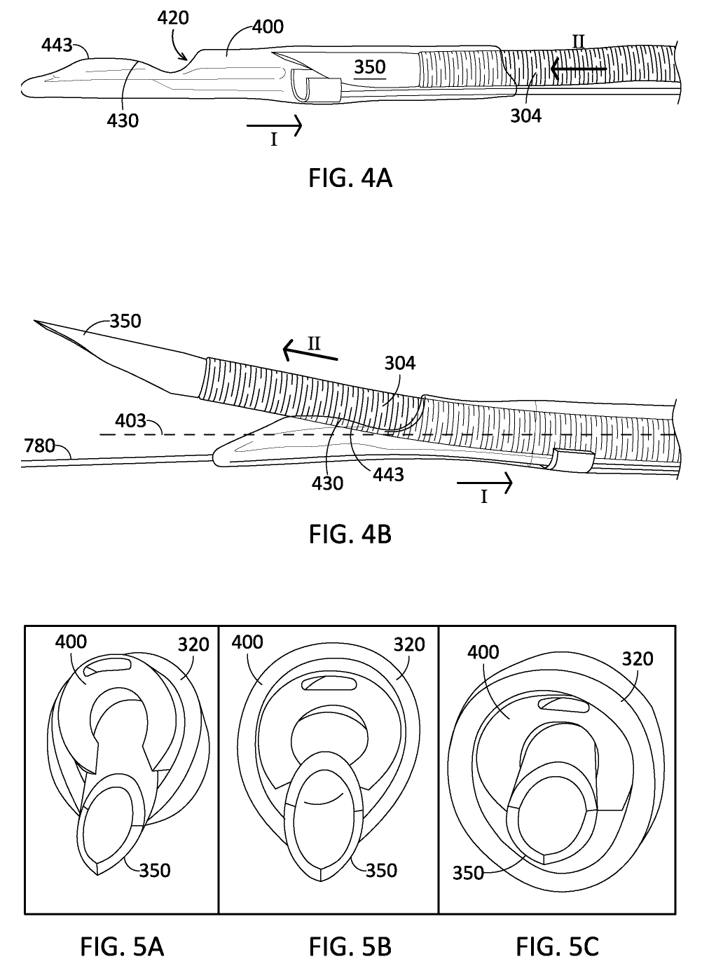

FIGS. 2B -D illustrate a perspective, side and top views of the distal portion 344 of the delivery catheter 304 of the delivery assembly 300, according to embodiments of the invention. The delivery assembly 300 comprises the delivery catheter 304 and the penetrating element guard or guard member 400. In some embodiments, the distal portion 344 of delivery catheter 304 comprises an elongated tubular shaft 344a and a distal end 344b, where the distal end 344b is coupled to or comprises the penetrating element 350. As shown in FIGS. 2A-D, the guard member 400 is configured to cover the penetrating element 350; the penetrating element 350 disposed within the guard member 400 is further illustrated in FIG. 4A. The guard member 400 covers the penetrating element 350 during navigation of the delivery catheter 304 through the patient’s vasculature to the target penetration site (e.g., a venous sinus wall). Further, the guard member 400 is configured to collapse around the shaft 344a of the delivery catheter 304 during withdrawal of the catheter 304, as described and depicted in FIGS. 5A-C In alternative embodiments, the guard member 400 may cover the penetrating element 350 during withdrawal of delivery catheter 304 after accessing the subarachnoid space or shunt deployment, thereby preventing inadvertent puncture or damage to other components of delivery assembly (e.g., guide catheter) and/or the patient’s vasculature. As shown in FIG. 2B, the delivery catheter 304 comprises a first lumen 305 configured to receive and/or accommodate a shunt. The guard member 400 includes a first lumen 405 configured to receive the penetrating element 350 and a second lumen 415 configured to receive and/or accommodate an elongate guide member 780 as shown in FIG. 2B.

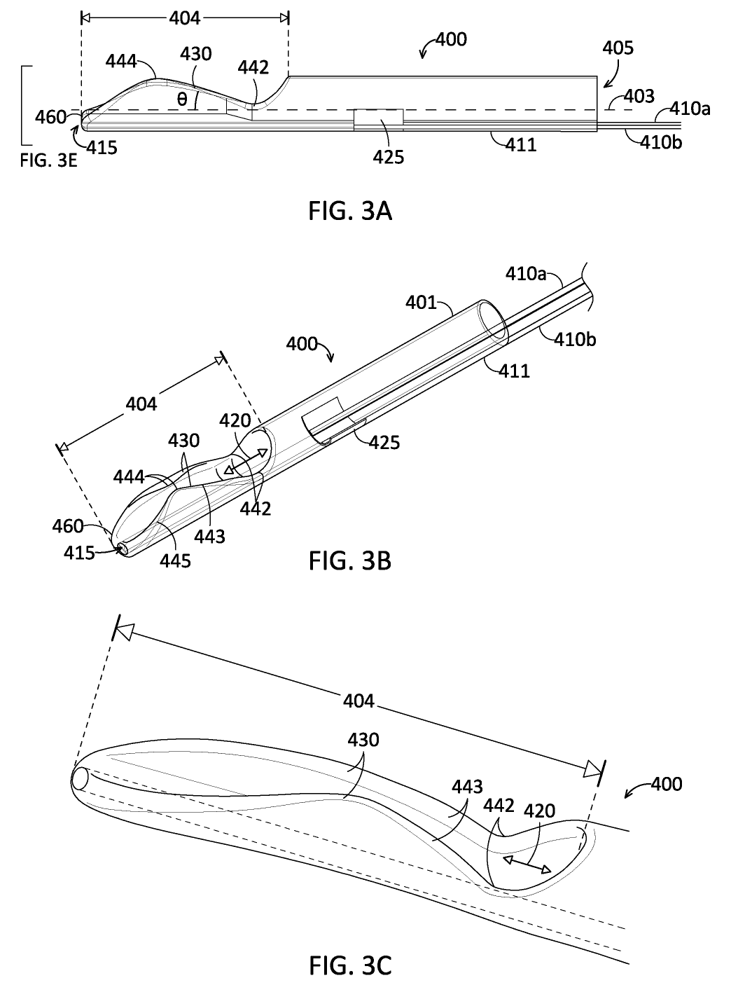

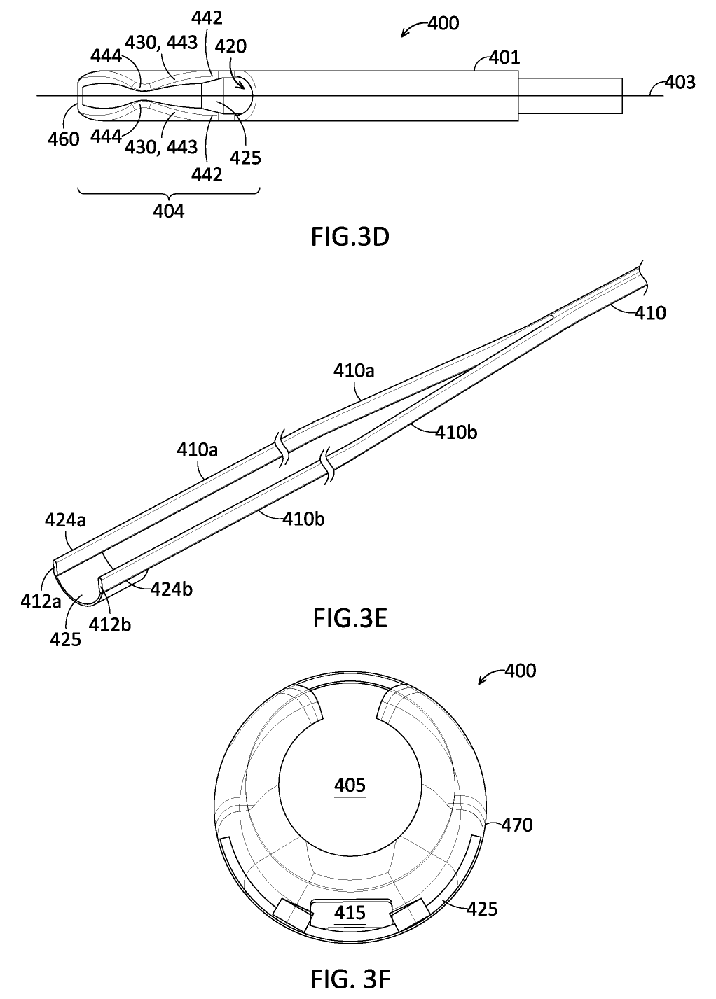

FIGS. 3A-F illustrate the guard member 400 according to embodiments of the inventions. The guard member 400 comprises a pull wire 410, the pull wire 410 bifurcates into two elongated portions 410a and 410b at a distal section 411 of the pull wire 410, as shown in FIG. 3E. The bifurcated pull wire portions 410a and 410b may be produced by laser cutting the distal section 411 of the pull wire 410, or any other suitable techniques. The two elongated portions 410a and 410b of the pull wire 410 are attached to an arcuate radiopaque marker 425 (e.g., partial or half cylindrical configuration, non-annular), as shown in FIG. 3E . The elongated portions 410a and 410bof pull wire 410 are embedded or encased within guard member 400 and include respective distal end tips 412a and 412b, each of the distal end tips 412a and 412b is coupled (e.g., attachment sections by welding or other suitable techniques) to the arcuate radiopaque marker 425 (FIG. 3E); the marker 425 is also embedded or encased within guard member 400.

As shown in FIGS. 2C-D, the delivery catheter 304 comprises a lumen 322 configured to receive and/or accommodate the pull wire 410. The delivery catheter 304 comprises a lubricious liner (e.g., PTFE or the like) covering the inner walls of the lumen 322 (not shown) to increase the lubricity and/or decrease friction of the pull wire 410 within the lumen 322 of the delivery catheter 304, thereby facilitating smooth proximal and/or distal movement, translation and/or actuation of guard member 400 to expose and re-cover the penetrating element 350.

Embodiments of the pull wire 410 of FIGS. 3A-F are configured to translate the guard member 400 proximally and/or distally relative to the delivery catheter 304 to at least partially expose or cover, respectively, the penetrating element 350. The bifurcated pull wire portions 410a and 410b are further configured to evenly transfer translation forces (e.g., pull and/or push) to the guard member 400, since the bifurcated portions 410a and 410b are coupled to substantially opposing lateral sides of guard member 400. As a result, the pull wire 410 provides smoother torque, advancement and/or retraction of the guard member 400 over the penetrating element 350 compared to a single pull wire without distal bifurcated portions 410a and 410b or two separate pull wires extending from guard member 400 to the proximal end of the delivery catheter 304. For example, the pull wire 410 with bifurcated portions 410a and 410b provides a desirable 1:1 torque response between the shaft 344a of the delivery catheter 304 and the guard member 400. The elongated portions 410a and 410b of bifurcated pull wire 410 are radially disposed within the guard member 400, thereby maintaining torque response between the guard member 400 and the shaft 344a of the delivery catheter 304 when the delivery catheter 304 is rotated. Additionally, the bifurcated pull wire portions 410 and 410b are configured to provide column strength to the delivery assembly 300 during advancement of the delivery catheter 304, avoiding and/or minimizing inadvertent proximal translation of the guard member 400 and undesirable exposure of the penetrating element 350. Further, the bifurcated pull wire portions 410a and 410b are configured to resist undesirable guard member 400 rotation around the distal portion 344 of the delivery catheter, thereby reducing the likelihood that the arcuate marker 425 may provide a false indication of delivery catheter 304 orientation in the vasculature. When the target penetration site is reached, thepull wire 410 is configured to proximally translate (e.g., pull) the guard member 400 to expose the penetrating element 350, as described and depicted in further detailed in FIGS. 4A-B.

Referring back to FIGS. 3A-F, the pull wire 410 with bifurcated portions 410a and 410b allow for a reduced diameter in the lumen of the delivery assembly, compared to having multiple pull wires for the guard member 400. Further, the bifurcated pull wire portions 410a and 410b reduce production time and cost of materials during manufacturing, since the pull wire 410 may be manufactured by laser cutting and splitting the distal section 411 of a single pull wire compared to manufacturing two separate pull wires coupled to the guard member 400 that extend between distal and proximal portions of the catheter (not shown).

The pull wire 410 can be composed of uncoated stainless steel, coated (e.g., PTFE) stainless steel or other suitable coated or uncoated materials. A PTFE or other lubricious coating, liners or the like, on pull wire 410 and/or in delivery catheter lumen 322 can increase the lubricity of the wire within the delivery catheter 304, thereby facilitating smooth proximal and distal actuation of guard member 400 to expose and re-cover the penetrating element 350. While the pull wire 410 embodiment depicted in FIGS. 3A-F, including sections 412aand 412b, have respective rectangular profiles (e.g., flat wire configuration), other pull wire embodiments can include non-rectangular cross-sectional profiles (e.g., circular, crescent). The pull wire 410 with bifurcated portions 410a and 410b, including the arcuate radiopaque marker 425, minimizes the overall profile of the guard member 400 by having less material (e.g., flat bifurcated wire and non-annular marker) and consequentially minimizing the French size of the delivery catheter 304.

The arcuate radiopaque marker 425 can comprise platinum-iridium 90/10 alloy or other suitable materials that provide sufficient radiopacity. Further, the arcuate radiopaque marker 425 allows the connection areas 424a and 424b to be coupled to the respective elongated portions 410a and 410b of the pull wire 410, as shown in FIG. 3E. The two elongated portions 410aand 410bof the pull wire 410, each comprises respective distal end tips 412a and 412b; the distal end tips 412a and 412b are coupled (e.g., attached, welded) to the connection areas 424aand 424b of the arcuate radiopaque marker 425. It should be appreciated that in embodiments where the pull wire 410 has a lubricious coating (e.g., PTFE), the distal end tips 412a and 412b do not include coating; the uncoated distal end tips 412a and 412b of the pull wire 410 allow for a weld or other attachment at the connection areas 424a and 424b of the radiopaque marker 425.

As stated above, the arcuate radiopaque marker 425 comprising partial or half cylindrical configuration (FIGS. 3A-B and 3E-F) minimizes the profile of the guard member 440. Further, the arcuate marker 425 is configured to determine the location (e.g., by tracking the marker position and orientation within the guard member 400) of the concentrically disposed penetrating element 350, when alignment of the arcuate marker 425 with a radiopaque marker 354 of the penetrating element 350 (FIGS. 2B, 4A and 6A) occurs. In some embodiments, the alignment of the guard marker 425 and penetrating element marker 354 allows the clinician to determine the location of the penetrating element 350 within the guard member 400 during tracking (e.g., navigation of the delivery assembly 300 through tortuous anatomy towards the target penetration site) so that the clinician may monitor or correct inadvertent withdrawal of the guard member 400 and/or deployment, orientation, and/or trajectory of the penetrating element 350. Additionally, the arcuate marker 425 and penetrating element marker 354 are configured to improve visualization of the delivery catheter 304 under fluoroscopy, since the arcuate marker 425 allows the clinician to determine orientation of the delivery catheter 304 and penetrating element 350 relative to a target penetration site in the vasculature; as opposed to an annular marker (not shown). As shown in FIG. 3F, the arcuate marker 425, embedded within the guard member 400, is adjacently disposed to an outer surface 470 of the guard member 400, such that the second lumen 415 of the guard member 400 is partially surrounded by the arcuate marker 425. In this embodiment, the arcuate marker 425 is configured to provide structural strength to the guard member 400, further avoiding and/or minimizing undesirable tear of the guard member 400 by the elongate guide member 780 translating within the second lumen 415 of the guard member 400. With improved structural strength of the guard member 400, including second lumen 415, embodiments of the delivery catheter 304 do not need a separate, dedicated lumen to receive the elongate guide member 780, for example, as disclosed in U.S. Patent No. 10,272,230. Removal of a lumen in the delivery catheter 304 for receiving an elongate guide member can streamline the delivery catheter 304 manufacturing steps and reduce the overall French profile of the delivery catheter 304 and/or delivery assembly 300. In addition, the delivery assembly 300 usability is improved because an operator is required to thread elongate guide member 780 through only one lumen (e.g., second lumen 415 of the guard member 400) before advancing delivery catheter 304 into the patient at the venous access site toward a target penetration site in the vasculature.

As better appreciated in FIG. 3F depicting a cross-sectional view of FIG. 3A, the guard member 400 comprises the first lumen 405 configured to receive and/or accommodate the penetrating element 350, and the second lumen 415 configured to receive and/or accommodate the elongate guide member 780. The guard member 400 is further configured to allow rapid exchange with the elongate guide member 780, a distal portion of elongated guide member 780 is coupled to a stent anchor or other temporary anchoring element (not shown).

Referring back to FIG. 2B the guard member 400 is disposed in a delivery configuration with respect to the distal portion 344 of delivery catheter 304, thus covering penetrating element 350 (also shown in FIG. 4A). The ppenetrating element 350 is positioned within lumen 405 of the guard member 400 and aligned with the radiopaque marker 425 of the guard member 400 such that, under fluoroscopy, the distal tip of penetrating element, for example, as confirmed by penetrating element marker 354, overlies radiopaque marker 425. The guard member 400 can be approximately 0.5’’ (1.27 cm) long or other suitable dimensions sufficient to cover penetrating element 350 on the distal portion 344 of the delivery catheter 304. The guard member 400 and the delivery catheter 304 including the penetrating element 350 are slidable relative to each other. For example, the guard lumen 405 is sized to allow guard member 400 to retract proximally over the penetrating element 350 and the distal portion 344 of the delivery catheter 304. For example, the inner diameter of guard lumen 405 can be approximately 0.0385’’ (0.09779 cm).

As depicted in FIGS. 3A-B, the marker 425 and the pull wire bifurcated portions 410a and 410b are embedded or encapsulated within the wall of guard member 400. Guard member 400 is composed of polymeric materials such as polyether block amide (Pebax® available from Arkema Group), HTPE, PTFE, Nylon, polyurethane, urethanes or any other polymeric material. Pebax® embodiments of guard member 400 can range from 27D to 70D hardness (e.g., Pebax® 63D). The wall thickness of guard member 400 can vary depending on top-to-bottom orientation of the guard.

As illustrated in the side view of the guard member 400 in FIG. 3A, the guard member 400 comprises a distal portion 404 having a contour/profile similar to a one period sine function with a peak 444 (e.g., maximum) and a valley 442 (e.g., minimum). It should be appreciated that the opposite side view (not shown) of the distal portion 404 of the guard member 400 of FIG. 3A comprises a contour/profile with a mirror-image one period sine function. The contour/profile in distal portion 404 of guard member 400 can facilitate delivery catheter 304 access to tortuous anatomy where the distal portion 404 of guard member 400 first accesses and advances through a narrowing or sharp bend, thereafter allowing the distal portion 344 of the delivery catheter 304 to track and follow through the narrowing or bend.

As shown in the perspective views (FIGS. 3B-C) and the top view (FIG. 3D) of the guard member 400, the guard member 400 comprises a tubular structure 401 having an aperture 420 at the distal portion 404 of the guard member 400. The distal portion 404 of guard member 400 defines the aperture 420 by having the respective peaks 444 of the sine functions to gradually approach each other, and the respective valleys 442 of the sine functions to gradually separate from each other. The split shape of the distal portion 404 of guard member 400 and convergence of the aperture 420 transitioning from valleys 442 to peaks 444 of a deflecting element 430 (better appreciated in FIGS. 3C-D) may be formed by laser cut of the tubular structure 401, molding or any other suitable technique. The shape of the distal portion 404 of guard member 400 creates an atraumatic tip 460 and further facilitates access to narrow or tortuous vasculature as the clinician navigates the delivery assembly toward the target penetration site. For example, the IPS diameter at the ostium in the internal jugular vein is often smaller than the IPS diameter at a target penetration site in the vessel (e.g., axial level of the jugular tubercle) and the operator must track delivery catheter 304 through the ostium to target penetration site for accessing the subarachnoid space. The contour/profile in distal portion 404 of guard member 400 initiates access of distal portion 344 of the delivery catheter 304 through the ostium into the vessel, which allows the remainder of the device to advance distally toward the target penetration site.

Further, the split shape of the distal portion 404 of guard member 400 comprises arcuate slopes 443 between the valleys 442 and the peaks 444, where the arcuate slopes 443 (FIG. 3B) form the deflecting element 430 configured to deflect the penetrating element 350 away from the elongate guide member 780 (FIG. 4B) and toward the penetration site. In some embodiments, the deflecting element 430 may be formed with steeper, or less steep, slopes 443 by varying the height of the peaks 444, depth of the valleys 442 and/or the angle “θ” (FIG. 3A) between deflecting element 430 and the longitudinal axis 403 parallel to the lumen 415 of the guard member 400 (FIGS. 3A-C). The angle “θ” of the slopes 443 that form the deflecting element 430 may range from about five degrees to about 40 degrees, or more. Increasing the angle “θ” of the slopes 443 relative to the longitudinal axis 403 of the guard member 400, increases the distance or separation between the penetrating element 350 and guide member 780, thus increasing the angle of penetration of penetrating element 350 with respect to the target site. In turn, greater separation between the penetrating element 350 and guide member 780 improves penetrating element 350 engagement with an IPS wall to ensure that the penetrating element 350 accesses the SAS, and can be particularly advantageous in vascular anatomies that do no exhibit sufficient curvature about a target penetration site to enable penetrating element 350 to travel off-axis from the guide member 780 and access the SAS.

FIGS. 4A-B illustrate an interface between the penetrating element 350 and guard member 400, according to embodiments of the invention. The guard member 400 is disposed over penetrating element 350 (FIG. 4A) to guard against inadvertent punctures in the vasculature while tracking the delivery catheter to the target penetration site. The guard member 400 may translate proximally over the distal portion of the delivery catheter 304 and the penetrating element 350 (shown by arrow I). Additionally, the penetrating element 350 may translate distally towards the deflecting element 430 of the guard member 400 (shown by arrow II) to expose the penetrating element 350 at the target penetration site (FIGS. 4A-B). For example, the clinician may retract the guard member 400 proximally, so that the penetrating element 350 disposed at the distal portion of the delivery catheter 304 contacts the deflecting element 430 / slopes 443 of the guard member 400, thus deflecting the penetrating element 350 away from the longitudinal axis 403 of the guard member 400 and elongate guide member 780 (FIG. 4B). In alternate embodiments of the delivery catheter 304, the clinician may advance the penetrating element 350 distally to contact the deflecting element 430 / slopes 443 of the guard member 400.

FIGS. 5A-C illustrate another view of the interface between the penetrating element 350 and guard member 400, according to embodiments of the invention. For example, FIGS. 5A-C show the interface between the penetrating element 350 and guard member 400 after the guard member 400 has been withdrawn proximally to expose the penetrating element 350 and/or the penetrating element 350 has been advanced distally to contact the deflecting element 430 / slopes 443 of guard member 400. The split shape of the distal portion 404 of guard member 400 having the slopes 443 is configured to allow the deflecting element 430 to open up and collapse around the shaft 344a of the delivery catheter 304 when the penetrating element 350 of the delivery catheter is retracted into the guide catheter 320. FIGS. 5A-C depict a sequential down-the-barrel view where the delivery catheter 304 is retracted or withdrawn into guide catheter 320 (e.g., while withdrawing the delivery catheter 304 from the SAS) and the split shape of the deflecting element 430 opens up and allows for the penetrating element 350 to at least partially fit within the aperture 420, substantially maintaining the diameter of the guide catheter 320. In contrast, in embodiments where the deflecting element is a solid feature of guard member 400 (e.g., without splits, respective deflecting element 430 or slopes 443), when the delivery catheter 304 is retracted or withdrawn, the penetrating element 350 will be disposed adjacent (e.g., wedging) to a solid deflecting element of the guard member (e.g., double barrel) thus undesirably extending the diameter of the guide catheter (not shown); this undesirable guide catheter extension can cause inadvertent shunt 200 removal from the SAS if, prior to shunt deployment, the shroud 800 and shunt 200 are also wedged in the guide catheter opening by a solid deflecting element of the guard member.

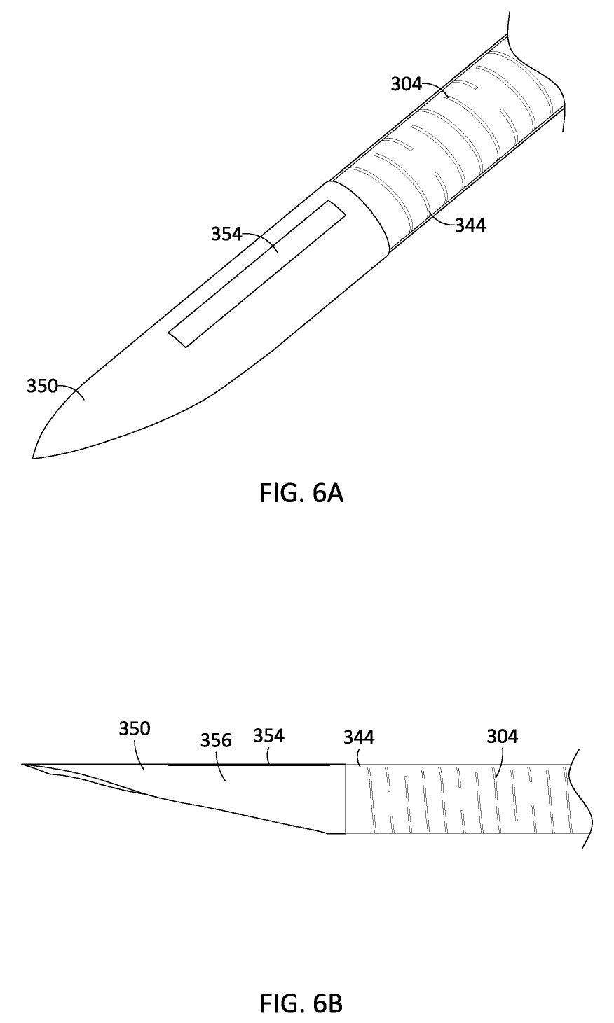

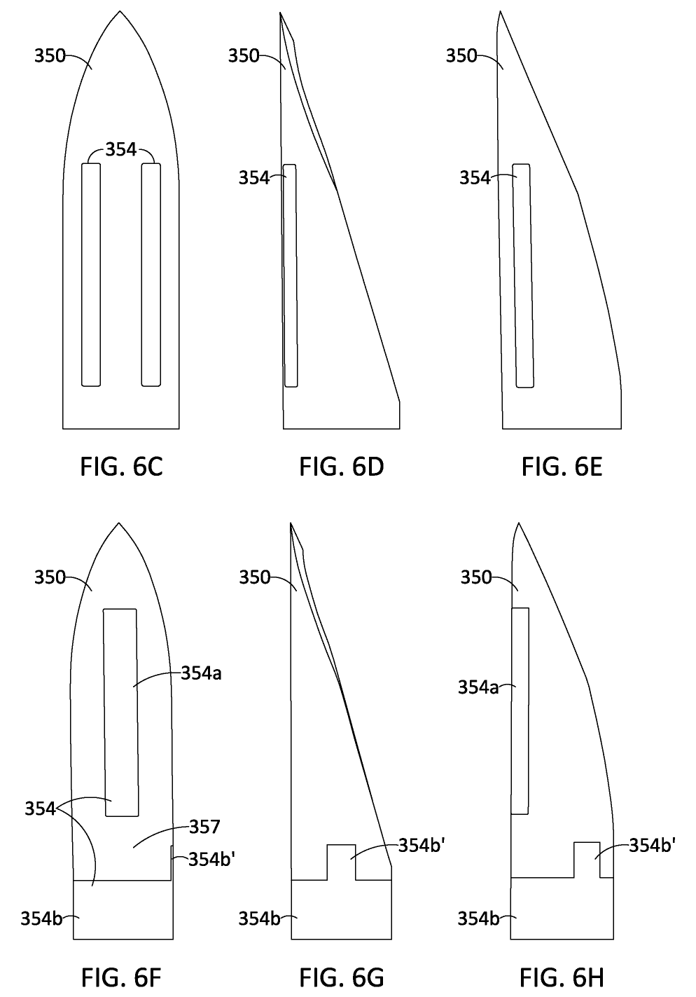

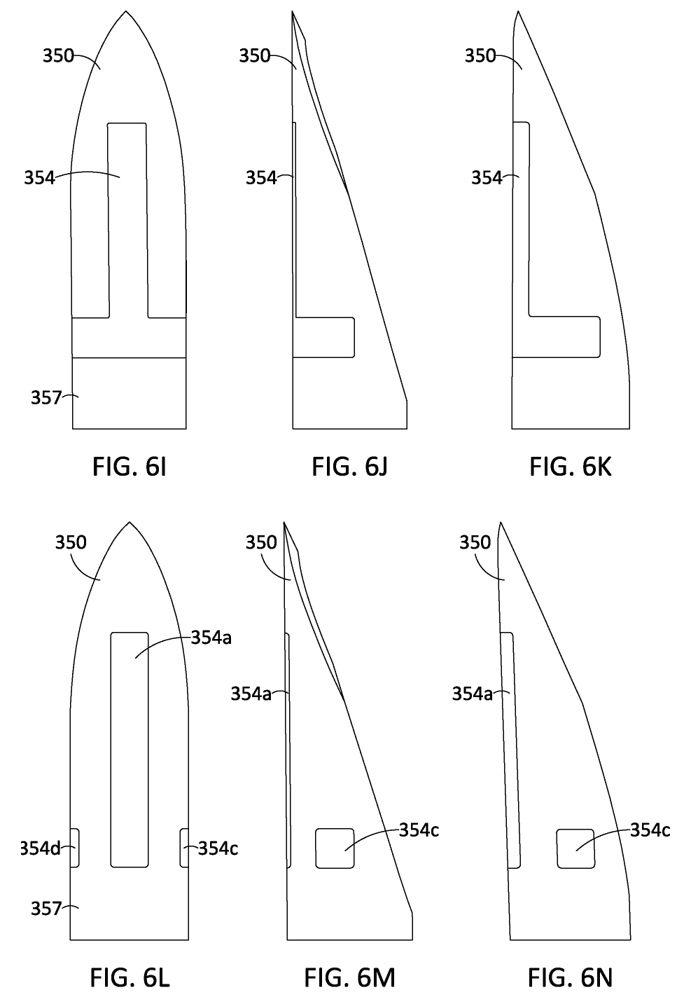

FIGS. 6A-N illustrate the penetrating element 350, according to various embodiments of the inventions. The penetrating element 350 comprises a sharp tapered, cannula-like end, bevel, pencil, or quincke tip needle, or the like. FIGS. 6A-B illustrate the penetrating element 350 disposed at the distal portion 344 of the delivery catheter 304. The penetrating element 350 further comprises a radiopaque marker 354 embedded therein, as better shown in FIG. 6A. FIGS. 6C-N illustrate alternative embodiments of the radiopaque marker 354 embedded in the penetrating element 350. The embedded marker 354 does not protrude from the outer surface 356 of penetrating element 350 (FIGS. 6A-N) or from the inner surface of the of penetrating element 350 (not shown), allowing smooth inner 356 and outer surfaces of the penetrating element 350. The radiopaque marker 354 may be embedded in a recess (not shown) of the outer surface 356 of penetrating element 350 and attached by welding or other suitable techniques. The radiopaque marker 354 may comprise platinum-iridium 90/10 alloy or other suitable materials that provide sufficient radiopacity. The radiopaque marker 354 is configured to allow the clinician to determine the location of the penetrating element 350 relative to an anatomic landmark (e.g., IPS or CP angle cistern), and further allow alignment with radiopaque marker 425 of the guard member 400, as shown in FIG. 2B. Improved visibility of the penetrating element 350 through the radiopaque marker 354 further mitigates the risks of the shunt being deployed in the subdural space or venous system and further, mitigating or avoiding the risks of injury to critical structures in the SAS by penetrating element 350 (e.g., brainstem, vertebral artery, basilar artery, anterior inferior cerebellar artery, cranial nerve VI (abducens nerve)). The radiopaque marker 354 facilitates visualization of the penetrating element 350 preventing inadvertent deployment of the penetrating element 350 through the guard member 400 when the clinician navigates the delivery assembly 300 through tortuous anatomy towards the target penetration site.

The radiopaque marker 354 may comprise an elongated configuration (e.g., longitudinally aligned with the tip of the penetrating element 350), as shown in FIGS. 6A-B. Alternatively, the radiopaque marker 354 may comprise a set of at least two elongated markers disposed substantially parallel to each other (e.g., off-set with respect to the tip of the penetrating element 350), as shown in FIG. 6C. Although FIGS. 6D-E show a single elongated marker 354, FIGS. 6D-E are side views of FIG. 6C having a set of two markers. FIGS. 6F-H illustrate another alternative embodiment of the radiopaque marker 354 of the penetrating element 350, where the radiopaque marker 354 comprises an elongated marker 354a and a annular marker 354b. The elongated marker 354a can be longitudinally aligned with the tip of the penetrating element 350, similar to FIG. 6A, while the annular marker 354b is disposed at a proximal portion 357 of the penetrating element 350. The annular marker 354b comprises a side portion 354b’ (e.g., square, rectangle or other suitable shape) as shown in FIGS. 6F-6H. FIGS. 6I-6K illustrate yet another embodiment of the radiopaque marker 354 of the penetrating element 350, where the radiopaque marker 354 comprises a “T” shape. The “T” shape of the marker 354 has the base of the “T” longitudinally aligned with the tip of the penetrating element 350, and the top of the “T” disposed in a curved, semi-annular configuration at the proximal portion 357 of the penetrating element 350, as shown in FIGS. 6I-6K. FIGS. 6L-6N illustrate another alternative embodiment of the radiopaque marker 354 of the penetrating element 350, having an elongated marker 354a, similar to FIG. 6F, and two side markers 354c and 354d disposed at the proximal portion 357 of the penetrating element 350.

The embodiments of the radiopaque marker 354 shown in FIGS. 6A-6N are configured to provide visual feedback regarding the penetrating element 350 orientation in the vasculature, which facilitates visualization and prevents inadvertent deployment of the penetrating element 350 through the guard member 400. The various embodiments of the radiopaque marker 354 facilitate visualization of the penetrating element 350 about a target penetration site, and visualization of the trajectory of penetrating element 350 into the SAS. For example, the marker 354b and portion 354b’ (FIGS. 6F-H), the top “T” shape of marker 354 (FIGS. 6I-K). and markers 354c and 354d (FIGS. 6L-N) may allow the clinician to determine the location of the penetrating element 350 relative to the radiopaque marker 425 of the guard member 400, while allowing the clinician to determine the orientation of the penetrating element 350 within the guard member 400 and in the vasculature.

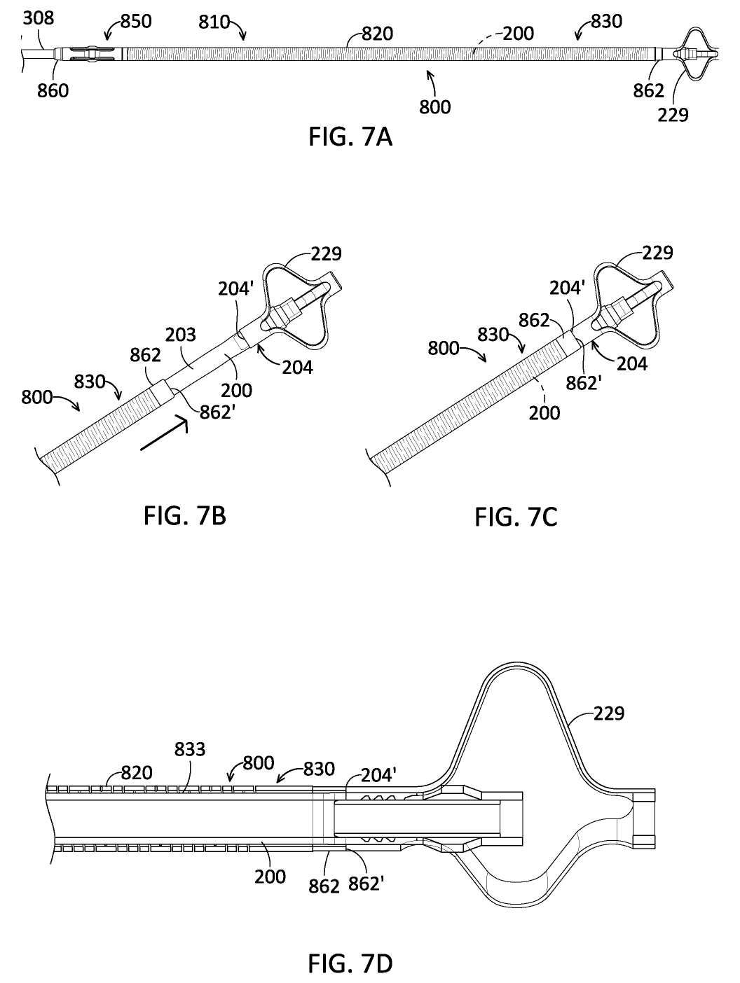

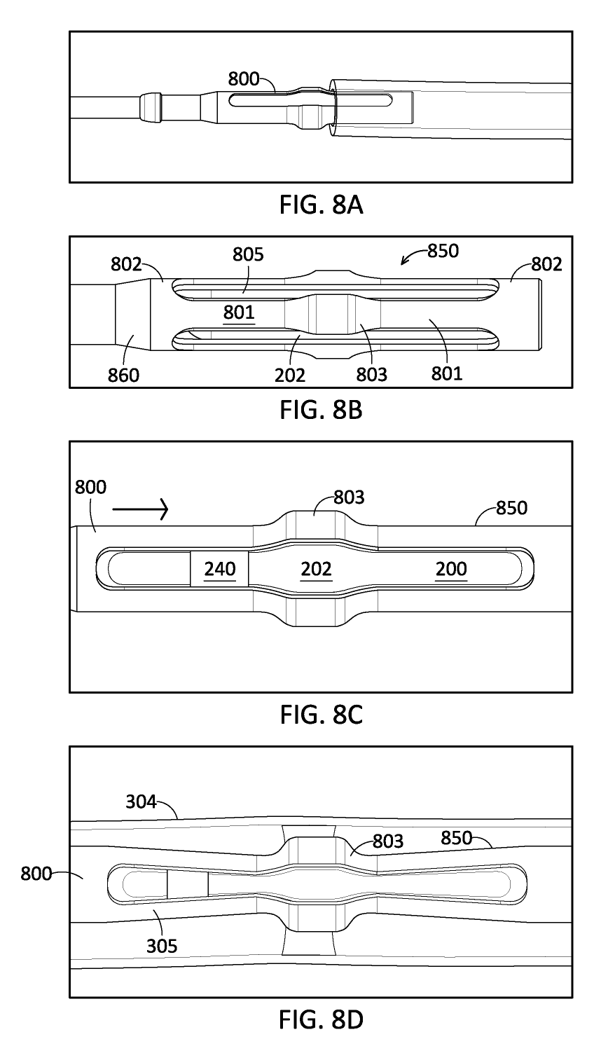

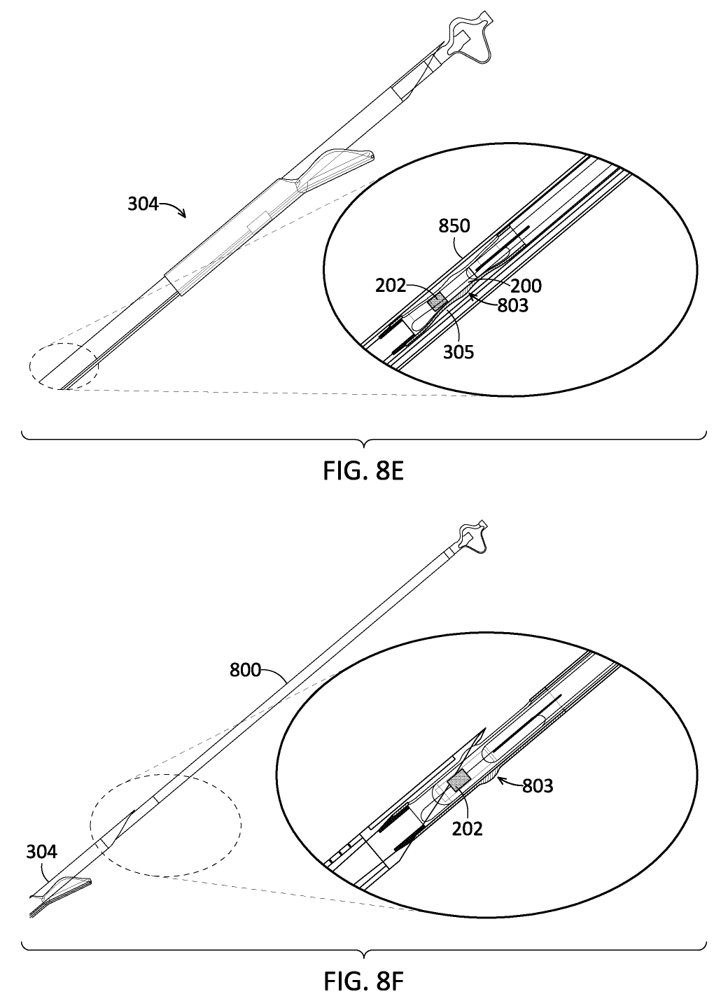

FIGS. 7A-D and FIG. 8A -D illustrate the implant (e.g., shunt) and the shroud according to the embodiments of the inventions. FIG. 7A shows the combined shunt 200 and shroud 800, FIGS. 7B-D. show the interface between the distal portion of shroud 800 and the distal portion of the shunt 200, while FIGS. 8A-D show the interface between the proximal portion of shunt 200 and the proximal portion of shroud 800 and a shroud proximal retention feature 850. The shroud 800 is configured to be at least partially disposed over and movable relative to the shunt 200 (FIGS. 7B-C). The shroud 800 is configured to minimize friction on and/or stretching of the shunt 200 to avoid or mitigate the risk of failure (e.g., tearing of the polymeric shunt body, removal of heparin or other coating on the shunt body, damage to the shunt valve, or the like) of the shunt 200. In addition, the disclosed embodiments of the shroud 800 provide a smoother and more controlled release of the shunt 200 because the shroud 800 does not capture a stretched, “necked down” (e.g., temporarily reduced outer diameter due to shunt body stretching and elongating) compared to shroud embodiments (e.g., shunt delivery shuttle embodiments disclosed in U.S. Patent App. Pub. No. US20200030588A1) that encapsulate a stretched, elongated shunt 200.

The shroud 800 comprises a tubular body 820 having a proximal portion 810, a distal portion 830, and a lumen 833 extending therebetween, as shown in FIGS. 7A-D. The shroud 800 further comprises a proximal marker 860 and a distal marker 862, as shown in FIG. 7A. The proximal and distal markers 860 and 862 are composed of tantalum, or any other radiopaque material, suitable for visualization of the shroud 800 under fluoroscopy. Particularly, the proximal and distal markers 860 and 862 are configured to allow visualization of the shroud 800 relative to the shunt 200 during advancement, deployment and/or delivery of the shunt 200 at the target site. The shroud distal marker 862 comprises an annular configuration, while the shroud proximal marker 860 comprises an annular tapered configuration (FIG. 7A) for gradually leading the shroud 800 (e.g., when the shroud 800 translates from within the penetrating element 350 to advance the shunt 200 into the target site). In alternative embodiments, the shroud proximal and distal markers 860 and 862 may comprise any suitable configuration.

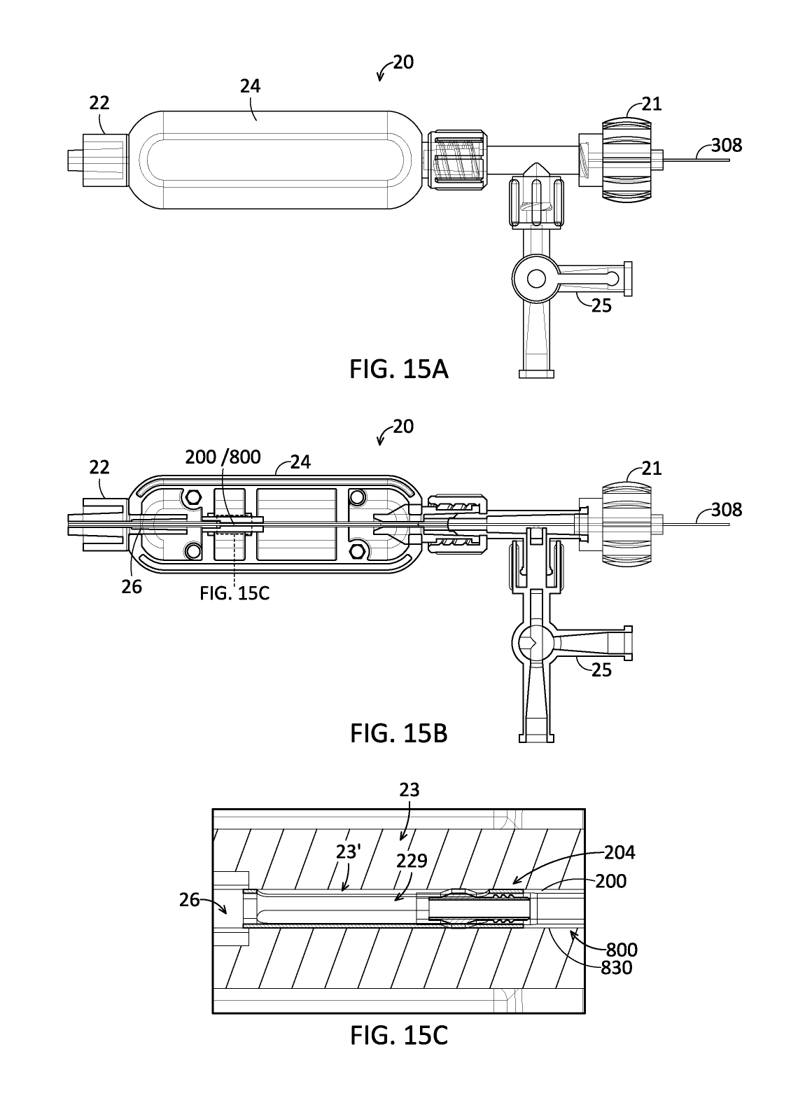

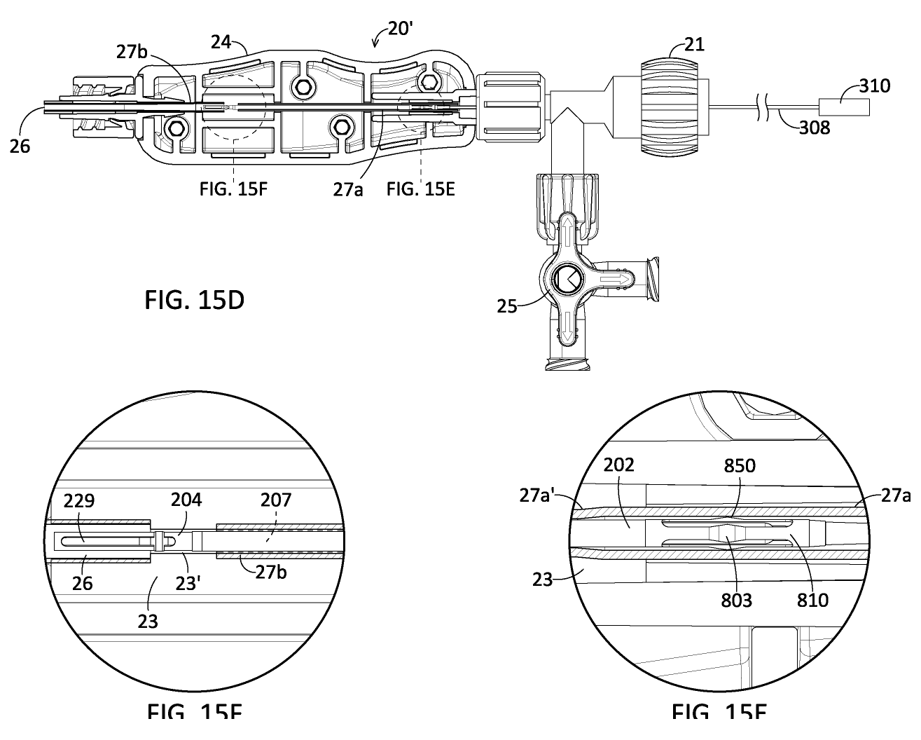

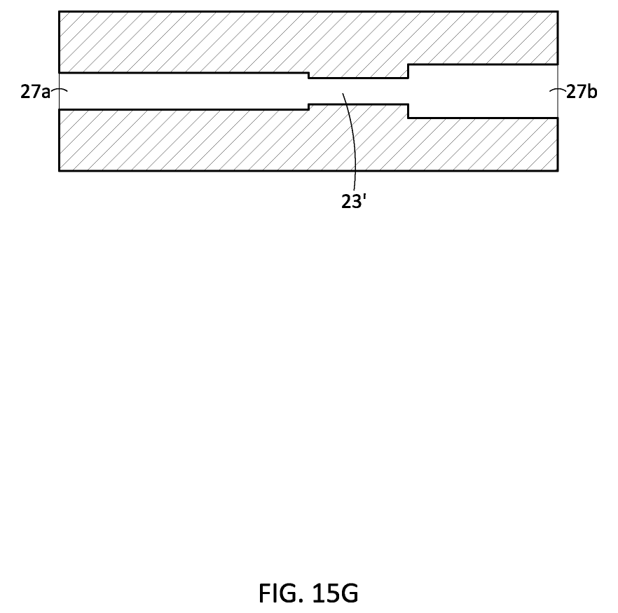

In FIG. 7A, the delivery wire 308 is shown proximately to the proximal marker 860 of the shroud 800. The delivery wire 308 is configured to orient, advance and/or retract the shroud 800 and shunt 200 through the first lumen 305 of the delivery catheter 304 (not shown). The delivery wire 308 comprises a suitable dimension (e.g., diameter of approximately 0.010’’ (0.254 mm) to 0.018’’ (0.4572 mm)) to navigate the shroud 800 and shunt 200 through the first lumen 305 of the delivery catheter 304 into the SAS. As known in the art, the delivery wire 308 comprises a tapered configuration for gradual stiffness (i.e., proximal section) to flexible (i.e., distal section) transitions, such that the delivery wire 308 transfers translational and rotational forces to orient, advance and/or retract the shroud 800 and shunt 200 including when distal portion 344 of delivery catheter 304 is disposed within tortuous anatomy. In some embodiments, the delivery wire 308 is further tapered along the length the shroud 800 to allow for smooth actuation of the shunt 200. Embodiments of the delivery wire can include a stop 310 (e.g., a bushing as shown in FIG. 15D) to prevent the operator from advancing the delivery wire too far distally and losing the proximal end of the delivery wire inside of the transfer device Tuohy borst during the procedure.

The tubular body 820 of the shroud 800 comprises super elastic Nitinol (or any other suitable super-elastic alloys or materials) having an interrupted spiral cut pattern (FIGS. 7A-D) or any other suitable cut pattern configured to provide flexibility and kink resistance to the shroud 800 during tracking through first lumen 305. In some embodiments, the shroud 800 includes at least one axial spine along the length of the tubular body 820. For example, the cut pattern of tubular body 820 includes an interrupted spiral cut leaving sequential uncut portions of the tubular body 820 forming an axial spine (not shown). In the embodiments of the shroud 800 having the at least one axial spine, the axial spine is configured to provide column strength to the shroud 800 to assist the shroud 800 with the translation of the shunt 200 through first lumen 305 of the delivery catheter 304. Additionally, the axial spine of the shroud 800 avoids or minimizes spring forces on the tubular body 820, such as when the shroud 800 transitions from being disposed within the delivery catheter 304 to being deployed out of the penetrating element 350; this advantageously prevents distal portion 830 of shroud 800 and/or shunt 200 from springing or “jumping” at the target deployment site (e.g., excessive and inadvertent distal movement of shunt 200) as the shroud 800 and shunt 200 are advanced through, free and clear of the penetrating element 350. Further, the cut pattern of the shroud 800 having the at least one axial spine is still configured to provide flexibility and kink resistance during tracking of the shroud 800 within the first lumen 305 of the delivery catheter 304. The embodiments of the disclosed inventions are an improvement, compared to shunt delivery shuttle embodiments disclosed in United States Patent App. Pub. No. US20200030588A1. Embodiments of shroud 800 prevent instances where the shunt cannot be released from the delivery assembly because the shroud resists potential kinking and crimping of the tubular body and lumen on shunt body that could prevent shunt from fully releasing from shroud.

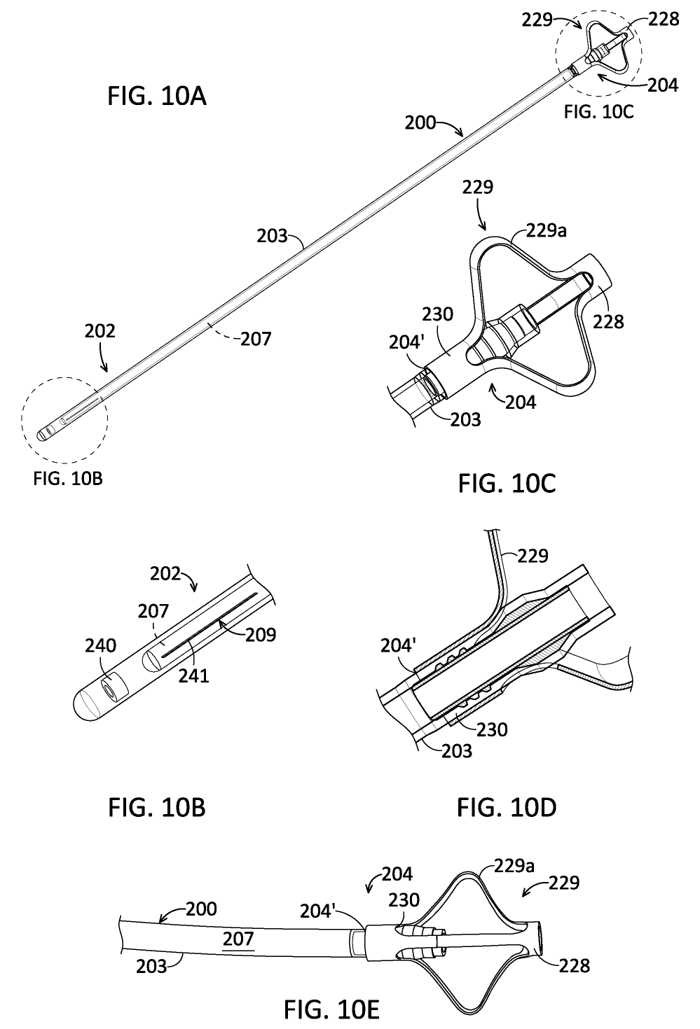

The shroud 800 is configured to slide over the shunt 200 and distally engage the shunt (FIGS. 7B-C) to translate and advance the shunt 200 through the first lumen 305 and penetrating element 350 of the delivery catheter 304 towards the target implantation site. Further, the shroud 800 is configured to engage (e.g., contact, abut, grasp) a proximal portion 202 of the shunt 200 (FIGS. 8C-E) assisting with the controlled positioning and advancement (e.g., moving, translating, pushing) of the shunt 200 through the delivery assembly 300. FIG. 7B illustrates the shroud distal portion 830 sliding over an elongated body 203 of the shunt 200 and FIG. 7C illustrates the shroud 800 disposed over the shunt 200 towards an anchoring mechanism 229 of the shunt (e.g., malecot). As shown in FIGS. 7B and 10D-E, the shunt body 203 comprises an outer diameter “OD” smaller than the OD of the shroud 800, such that the shunt 200 is slidably disposed within the lumen 833 of the shroud 800, except that the OD of the anchoring mechanism 229 of the shunt 200 is substantially the same OD as the OD of the shroud 800. Further, the anchoring mechanism 229 of the shunt 200 comprises an annular edge 204’ configured to contact/abut an annular edge 862’ of the shroud 800 marker 862 (FIGS. 7B-D and 10D-E). During the engagement of the shroud 800 with the shunt 200, while the shroud 800 advances over the shunt 200, the annular edge 862’ of the shroud 800 marker 862 contacts/abuts the annular edge 204’ of the anchoring mechanism 229 at the distal portion 204 of the shunt 200 (FIGS. 7B and 10E). As better appreciated in FIGS. 7C-D, when the annular edge 862’ of the marker 862 is in contact with the annular edge 204’ of the anchoring mechanism 229 of the shunt 200, the OD of the engaged shroud 800 and shunt 200 is substantially the same, such as, the OD is flush, substantially flush, or even between the shroud distal portion 830 (e.g., distal marker 862) and the distal anchoring mechanism 229 of the shunt 200. Therefore, the distal portion 830 of the shroud 800, when engaged to the distal portion 204 of the shunt 200 at the distal anchoring mechanism 229, is configured to translate, advance, and/or push the shunt 200 through the delivery catheter 304 first lumen 305 and the penetrating element 350 for deployment and delivery in the target site.

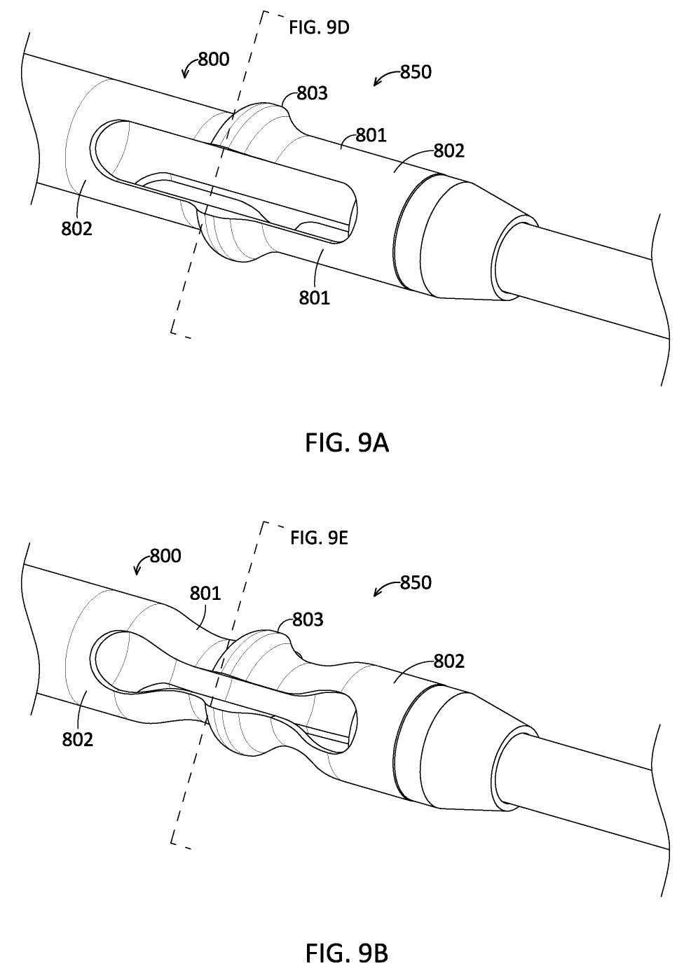

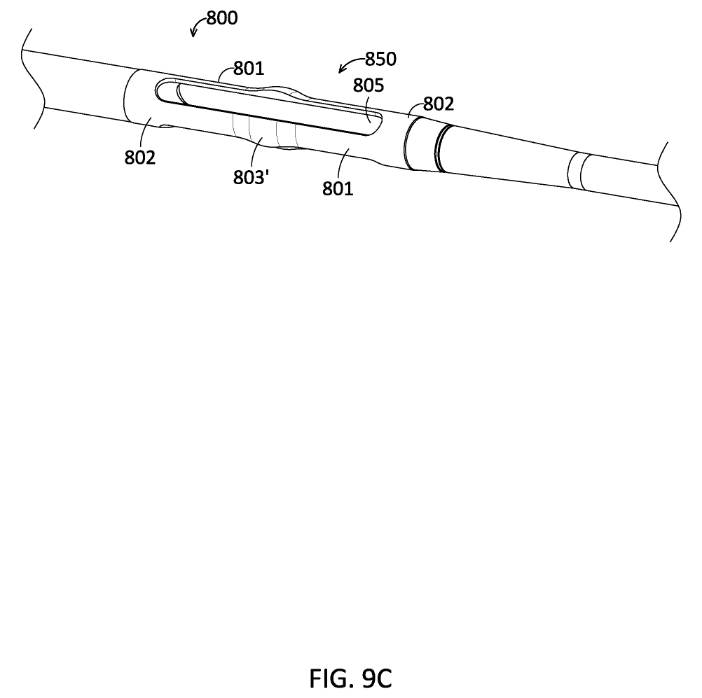

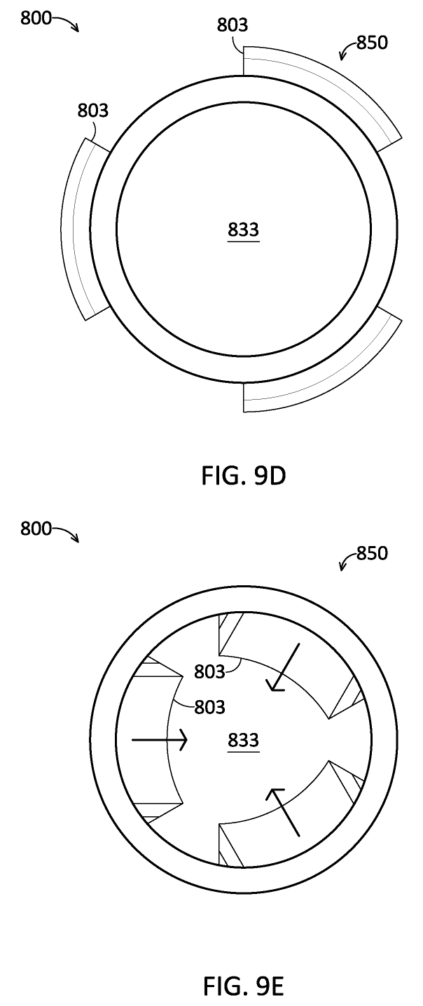

FIGS. 8A-F depict the actuation of embodiments of the shroud 800 with the proximal portion 202 shunt 200. The proximal portion 810 of the shroud 800 comprises a proximal retention feature 850 (FIGS. 7A, 8B-F and 9A-B). FIGS. 9A-E illustrate alternative embodiments of a proximal retention feature 850 of the shroud 800 in a relaxed/expanded configuration (FIG. 9A, 9C and 9D) and in a collapsed/contracted configuration (FIG. 9B and 9E). The proximal retention feature 850 is configured to hold, grasp and/or restrain the proximal portion 202 of shunt 200 to assist with the advancement of the shunt 200, for example, through the delivery catheter lumen 305 toward the target site. The proximal retention feature 850 is configured to slide over the proximal portion 202 of shunt 200 and the proximal portion 202 of shunt 200 is configured to be disposed within the retention feature 850 (FIG. 8B), such that the retention feature 850 grasps the proximal portion 202 of shunt 200 by assuming the retention feature 850 collapsed/contracted configuration (FIGS. 8D-E, 9B and 9E) (e.g., when the proximal portion 202 of shunt 200 and shroud retention feature 850 are constrained within delivery catheter first lumen 305). The proximal retention feature 850 comprises a plurality of elongated members 801 extending between two annular members 802, each of the plurality of elongated members 801 comprises a protrusion 803 disposed approximately in the middle of the respective elongated members 801, as better appreciated in FIGS. 8B and 9A-C.

FIG. 8E illustrates a detailed inside view of the delivery catheter 304, where the constrained retention feature 850 of the shroud 800 is engaged with shunt proximal portion 202 within the delivery catheter lumen 305. The proximal portion 202 of the shunt 200 is securely restrained by the elongated members urged toward the shunt by the constrained protrusions 803 of retention feature 850. While FIG. 8F illustrates a detailed view of the shunt proximal portion 202 and proximal retention feature 850 emerging from the delivery catheter 304, where the protrusions 803 of the retention feature 850 are unconstrained from the shunt proximal portion 202 upon deployment from the delivery catheter penetrating element 350.

FIG. 9C illustrates an alternative embodiment of FIGS. 9A-B, where the protrusion 803’ of FIG. 9C comprises a smaller radius, thickness and/or surface area (as further illustrated in FIGS. 8E-F) than the protrusion 803 of FIGS. 9A-B. Variations on the radius, thickness and/or surface of the protrusion 803 of the retention feature 850 of the shroud 800 depends on the desired retention (e.g., compression force exerted to the shunt or other device) needed on the shunt 200 disposed within delivery catheter 304 first lumen 305. It should be appreciated that a larger radius, thickness and/or surface of the protrusion 803 will exert greater compression force on the shunt 200, when the shunt 200 is disposed within the shroud lumen 833, and the shunt 200 and shroud 800 are disposed within delivery catheter 304 first lumen 305 (FIG. 8D).

In further alternative embodiments, one or more protrusions 803 may be disposed along any suitable portion of their respective elongated members 801 of the proximal retention feature 850. The protrusions 803 of the proximal retention feature 850 are configured to gradually lead the shroud 800, when the shroud 800 translates within the delivery catheter first lumen 305 and through the penetrating element 350 to advance the shunt 200 into the target site. Additionally, the proximal retention feature 850 comprising the plurality of elongated members 801 have less material (e.g., slots 805 between the elongated members 801 in FIG. 8B) as opposed to a cylindrical retention feature with constant surface area (not shown). The proximal retention feature 850 having less material or surface area is configured to minimize friction between the shroud 800 and the penetrating element 350, which facilitates advancement of the shroud 800 through the delivery catheter lumen 305 when the catheter is disposed within tortuous anatomy.

The shroud 800 may be composed of titanium, tantalum, stainless steel, Nitinol, or other super-elastic alloys or materials. In some embodiments, the shroud 800 may be manufactured by cutting, electropolishing, and/or welding suitable materials and components to produce the shroud 800. For example, the retention feature 850 may be manufactured by laser cutting a tubular member, or by welding the plurality of elongated members 801 into the two annular members 802.

FIGS. 9D-E illustrate cross-section views of the proximal retention feature 850 of FIGS. 8F and 8E, respectively. As shown in FIG. 9D, the protrusions 803 of retention feature 850 of the shroud 800 are in a relaxed, expanded or unconstrained configuration, and the lumen 833 of the shroud 800 is unobstructed by the protrusions 803. In the unconstrained configuration, the protrusions 803 will not grasp or compress the shunt proximal portion and the shunt, which will be freely movable within the shroud lumen 833. The unconstrained configuration facilitates loading the shunt into the shroud 800, for example, during device packaging and sterilization, and deployment of the shunt at the target location (FIG. 8F). As shown in FIG. 9D, the protrusions 803 of retention feature 850 of the shroud 800 are in a collapsed, contracted or constrained configuration. When the protrusions 803 are constrained (e.g., by advancing the shroud 800 and shunt 200 distally through a lumen 26 of the transfer device 24 and into the delivery catheter lumen 305), the protrusions 803 are biased into the shroud lumen 833. When constrained, the protrusions 803 grasp or compress onto the shunt proximal portion 202 (FIGS. 8D-E), preferably proximal to the shunt valve 209, which occurs when the shunt 200 and shroud 800 are advanced through the transfer device lumen 26, through handle 10, and into the first lumen 305 of delivery catheter 304. As shown by the arrows of FIG. 9E, the protrusions 803 are biased to be constrained radially inward when proximal retention feature 850 is constrained within the delivery catheter lumen 805. The retention feature 850 constrained configuration within the delivery catheter lumen 805 maintains positive shroud 800 engagement with the shunt 200 (FIGS. 8D-E which enables the delivery wire 803 to advance and retract the shunt 200 and shroud 800 within the delivery catheter lumen 305. When the shroud 800 and shunt 200 are advanced distally via delivery wire 308 through delivery catheter lumen 305 and out of the penetrating element 350 for shunt deployment, the retention feature protrusions 803 are no longer constrained by the inner diameter of delivery catheter lumen 305; the retention feature elongated members 801 expand radially to release retention feature 850 engagement with shunt proximal portion 202 (FIG. 8F). FIGS. 10A-E illustrate perspective, detailed and cross-sectional views of the shunt 200 (e.g., shunt), according to embodiments of the disclosed inventions. The shunt 200 comprises the proximal portion 202, a body portion 203, the distal portion 204 and a lumen 207 extending therebetween, as shown in FIG. 10A. As shown in the detailed view of FIG. 10B, the proximal portion 202 of the shunt 200 comprises a valve 209 and a radiopaque marker 240. The valve 209 is configured to be in fluid communication with the shunt lumen 207. In some embodiments, the valve 209 is a one-way valve comprising one or more slits 241. The marker 240 comprises a cylindrical band embedded in the proximal portion 202 of the shunt 200 (FIG. 8C and 10B). To improve engagement with proximal retention feature 850 and minimize impact to the valve 209 and CSF lumen 207 of shunt 200, the proximal tip of shunt 200 (e.g., the shunt proximal portion 202 proximal to the proximal most edge of shunt lumen 207, shown in FIG. 10B) can be a solid or substantially solid piece of polymeric material of shunt body 203 and the proximal marker 240. In alternative embodiments, the marker 240 may be swaged over the distal portion 204 of the shunt 200. The marker 240 is composed of any suitable radiopaque material and comprises a suitable surface area to assist visualization of the shunt 200 under fluoroscopy, avoiding or minimizing risks to patient during the accessing of the CP angle cistern.

The shunt 200 (e.g., proximal 202 and body 203 portions) includes elastomeric polymer(s) suitable for implant applications including, but not limited to, silicone, polyurethane, polycarbonate urethane, thermoplastic polyurethane, aromatic or aliphatic polycarbonate thermoplastic polyurethane, silicone/polyurethane blends (e.g., thermoplastic silicone polycarbonate polyurethane comprising 20% silicone copolymer), or polyurethane silicone blends (e.g., polyurethane silicone copolymer). The shunt 200 materials are selected to advantageously resists thrombus formation, particularly on the proximal portion 202 of the shunt 200. Optionally, the shunt 200 includes an anti-thrombotic coating to prevent thrombus formation including, but not limited to, heparin-based or phosphorylcholine-based anti-thrombotic coatings.

The distal portion 204 of the shunt 200 comprises the anchoring mechanism 229 (e.g., malecot) having a compressed configuration (FIG. 15C) for navigating through the delivery catheter 304 and an expanded configuration (FIGS. 7A-D, 10A, and 10C-E) for anchoring the shunt 200 at the target site in the patient. The anchoring mechanism 229 is composed of titanium, stainless steel, Nitinol, or other super-elastic alloys. The anchoring mechanism 229 comprises a proximal retainer element or collar 230, a distal radiopaque marker 228, and a plurality of elongated deformable elements 229a (e.g., arms) disposed therebetween (FIGS. 10A, and 10C-E). The proximal retainer element or collar 230 includes the distal anchoring mechanism 229, shunt distal portion 204 and annular edge 204’ described above in connection with FIGS. 7B and 10D-E. The proximal collar 230 and the distal marker 228 are composed of suitable radiopaque materials and can comprise an annular configuration. The deformable elements 229aare disposed radially outward in the expanded configuration of the anchoring mechanism 229. When shunt 200 is deployed out of the delivery catheter 304, the anchoring mechanism 229 transitions (e.g., self-expands) from the compressed configuration within the delivery catheter 304 to its expanded or deployed configuration. During deployment, under fluoroscopy, the clinician may observe the proximal collar 230 and the distal marker 228 move closer together, confirming that the anchoring mechanism 229 has properly transitioned to its the expanded or deployed configuration.

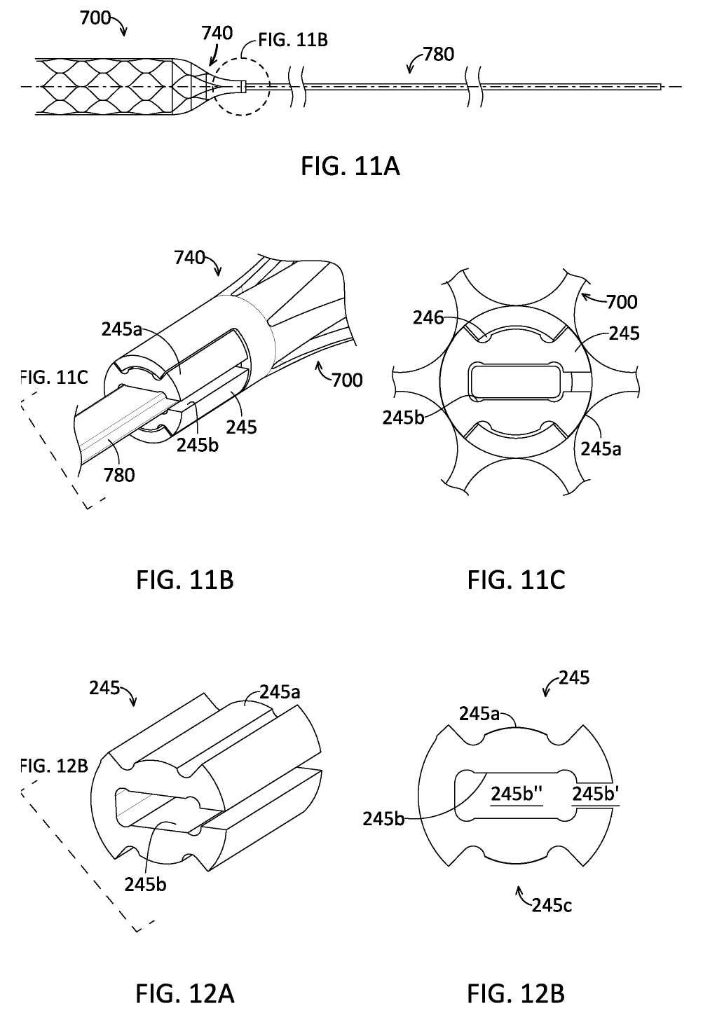

FIGS. 11A-C illustrate perspective and down-the-barrel views of the elongate guide member 780, an anchor 700 and a proximal marker 245, according to embodiments of the disclosed inventions. The elongate guide member 780 can have a flat, rectangular, or otherwise non-circular, cross-sectional profile, as shown for example in FIGS. 2A and FIG. 11B. As shown in the detailed view of FIG. 11B, the elongate guide member 780 is coupled (e.g., directly or indirectly, attached, secured, joined, or the like) to the proximal portion 740 of the anchor 700. The anchor 700 may be composed of shape memory, self-expandable and biocompatible materials, such as Nitinol^{®}, or other super-elastic alloys, stainless steel, or cobalt chromium, or combinations thereof, and comprises a stent-like configuration. In some embodiments, the anchor 700 may include materials that are compatible with magnetic resonance imaging and have radiopacity sufficient to allow the use of known imaging techniques. Alternatively, the anchor 700 can be composed of magnesium, zinc, or other bio-absorbable or dissolvable components. The anchor 700 further comprises a radially collapsed or delivery configuration (not shown) and, a radially expanded or deployed configuration (FIG. 11A). In the deployed configuration the anchor 700 is configured to radially expand and anchor itself within the IPS or CS, allowing tracking and advancement of the guard member 400, slidably disposed around the elongate guide member 780 (FIG. 4B).

The proximal portion 740 of the anchor 700 comprises a proximal marker 245, as shown in FIGS. 11A-B. FIGS. 12A-B illustrate perspective and down-the-barrel views of the proximal marker 245. The proximal marker 245 comprises an outer surface 245a including peaks and recesses, and an inner surface 245b defining an opening 245b’ and a lumen 245b’’ (FIGS. 11A-12B), the outer 245a and inner 245b surfaces form a contour 245c of the proximal marker 245, as better appreciated in FIG. 12B. The proximal marker outer surface 245a, inner surface 245b and contour 245c are configured to snuggly fit within the proximal portion 740 of the anchor 700, as shown in FIGS. 11A-B. The proximal marker 245 is configured to provide and maximize tensile strength when coupled to the anchor 700 and the elongate guide member 780. The proximal marker 245 is composed of tantalum or any other suitable radiopaque material. In some embodiments, the proximal marker 245 is manufactured by electrode wire cutting mechanism “EDM” programmed to form the contour 245c of the proximal marker 245. The EDM manufacturing process produces the proximal marker 245 having a larger volume compared to the prior art markers, which allows smaller gaps 246 between the proximal marker 245 and the proximal portion 740 of the anchor 700, as shown in FIG. 11B.

FIG. 13 illustrates a cross-sectional view of a handle 10 for the delivery catheter 304 of FIG. 1, according to embodiments of the disclosed inventions. The handle 10 is configured to be coupled to or integrated into the delivery catheter 304, in place of the Luer assembly 17 of FIG. 1. As shown in FIG. 13, the handle 10 comprises a Luer fitting 12, a delivery lumen flush port 14, a tether lumen flush port 16, a tether actuation mechanism 18, a strain relief 19 and the delivery catheter 304 proximal portion 324. The delivery lumen flush port 14 is in fluid communication with the first lumen 305 of the delivery catheter 304. The tether lumen flush port 16 is an optional feature of the handle 10; the tether lumen flush port 16 is in fluid communication with the delivery catheter lumen 322 that receives the pull wire 410 for actuating the guard member 400. In some embodiments, the flush ports 14 and 16 are one way lumen flush ports. During clinical use, the flush port 14 and the flush port 16 can be connected to pressurized saline bags to maintain fluid flow through the respective delivery catheter lumens 305 and 322 to prevent blood reflux into and the formation of air bubbles within such delivery catheter lumens. The tether actuation mechanism 18 is integrated into the handle 10, for actuating guard member 400. In some embodiments, the tether actuation mechanism 18 is separate from the handle (not shown).

FIGS. 14A-D illustrate perspective and cross-sectional views of an alternative embodiment of the handle of FIG. 13, according to embodiments of the disclosed inventions. As shown in FIGS. 14A-D, the handle 10’ comprises the same features as of the handle 10 (i.e., Luer fitting 12, delivery lumen flush port 14, tether retraction mechanism 18, strain relief 19 and delivery catheter 304 proximal portion 324) except that such handle 10’ does not include a separate, optional, tether lumen flush port. The tether actuation mechanism 18 is configured to turn, as shown by arrow 18a in FIG. 14B, so that the mechanism 18 is loosened from the handle 10’ to allow translational movement 18b, as shown in FIG. 14C. Further, the tether actuation mechanism 18 is configured to be pulled down, as shown by arrow 18b in FIG. 14C, so as to transfer translation forces to the pull wire 410 to actuate the guard member 400 proximally and expose the penetrating element 350 from the guard member 400. It should be appreciated that the tether actuation mechanism 18 of FIG. 13 is configured to be actuated in the way shown and described in FIGS. 14B-D. In addition, the tether actuation mechanism 18 of FIG. 13 can be configured to be actuated in the opposite direction shown and described in FIGS. 14B-D, as to transfer translation forces to the pull wire 410 to actuate the guard member 400 distally and cover the penetrating element 350 with the guard member 400. FIG. 14D illustrates a cross-sectional view of the handle 10’ of FIGS. 14A-C. As shown in FIG. 14D, the tether retraction mechanism 18 is coupled to the pull wire 410. In some embodiments, the proximal end of the pull wire 410 is fixedly coupled to the tether retraction mechanism 18.

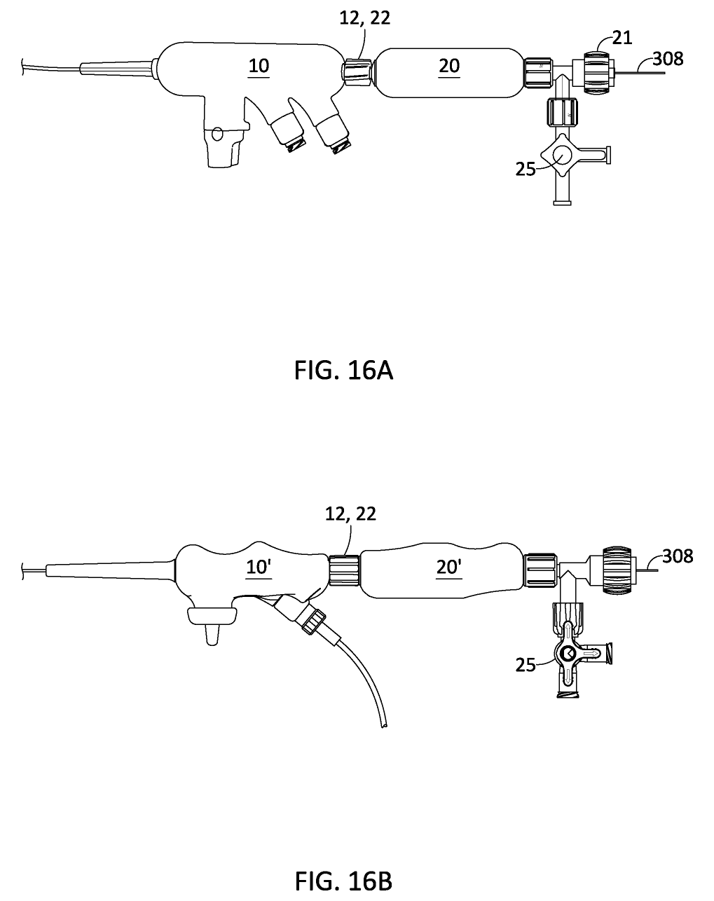

FIGS. 15A-C illustrate perspective and detailed views of a transfer device 20, according to embodiments of the disclosed inventions. FIGS. 15D-G illustrate perspective and detailed views of a transfer device 20’, according to embodiments of the disclosed inventions. The transfer devices 20/20’ are configured to transfer the shunt 200 and the shroud 800 into either handle 10 or 10’ (FIGS. 16A-B). As shown in FIG. 16A, the transfer device 20 is coupled to the handle 10, and as shown in FIG. 16B, the transfer device 20’ is coupled to the handle 10’. It should be appreciated that either transfer device 20/20’ is configured to be coupled to either handle 10/10’.

Each transfer device 20/20’ comprises a Touhy-borst adapter 21, a Luer fitting 22, a passive seal 23, a housing 24, and a stopcock 25, as shown in FIGS. 15A-B and 15D. The housing 24 comprises the lumen 26 extending therethrough, where the lumen 26 and passive seal lumen 23’ are configured to receive the shunt 200 and the shroud 800. As shown in FIGS. 15D-E, the transfer device 20’ comprises the same features of the transfer device 20, additionally the transfer device 20’ comprises a support member 27 disposed within the housing 24. The support member 27 is configured to provide column support to the housing 24, lumen 26 and/or shroud 800 for transferring of the shunt 200 and the shroud 800 through lumen 26 and passive seal lumen 23’ into either handle 10 or 10’. The support member 27 may comprise a metallic, polymeric or any other suitable material tube, elongated member, or the like. In the embodiments of FIGS. 15D-E, the support member 27 comprises a first support member 27a and a second support member 27b; FIG. 15G shows a cross-sectional view of first support member 27a, passive seal 23, and second support member 27b according to the transfer device embodiment of FIG. 15D. The support member 27a comprises an inner diameter (ID) between 0.0665 to 0.685 to mm; while the support member 27b comprises an ID between 1.30 to 1.42 to mm. It should be appreciated that the ID of the respective support members 27a/27bmay taper between the above-identified ranges or the ID may be constant (e.g., ID of 0.675 mm for support member 27a and ID of 1.37 mm for support member 27b).

As shown in FIGS. 15D-F, the shunt 200 and the shroud 800 are disposed within the lumen 26 of the housing 24, where the proximal portion 202 of the shunt 200 and the proximal portion 810 of the shroud 800, including the retention feature 850, are disposed within the first support member 27a of transfer device lumen 26 (detailed view in FIG. 15E), and the distal portion 204 of the shunt 200 and the anchoring mechanism 229 are disposed within the second support member 27b of transfer device lumen 26 and the passive seal lumen 23’ (detailed view in FIGS. 15F). In some embodiments, the support member 27a ID tapers down (e.g., tapers radially inward in a distal direction) towards the passive seal 23’ of the housing 24 (shown as taper 27a’ in FIG. 15E), such that the retention feature 850 of the shroud 800 (shown in a relaxed/expanded configuration in FIG. 15E), will assume the collapsed /contracted configuration (FIG. 9B and 9E) as the shroud is advanced distally through the taper of support member 27a. For example, the ID of the tubular support member 27a lumen decreases in a distal direction along a length of the distal portion, and such that, as the shroud 800 and shunt 200 are advanced distally through tubular support member 27a lumen, the shroud retention feature 850 is compressed to thereby secure or grasp a proximal portion 202 of the shunt 200. The support member 27a is configured to collapse, constrain and/or restrain the protrusions 803 of retention feature 850, as the shroud 800 and shunt 200 are distally or longitudinally translated/pushed through the lumen 26 of the housing 24, thereby securing the proximal portion 202 of the shunt 200 in the retention feature 850 of the shroud 800. The passive seal lumen 23’ and the ID of the support member 27b are also configured to maintained the collapsed/contracted configuration of the retention feature 850, such that the shroud 800 engages and secures the proximal portion 202 of the shunt 200 throughout the transfer through transfer device lumen 26 into the handle 10/10’. During clinical use, the Touhy-borst adapter 21 is configured to open and allow distal translation of the delivery wire 308, the shroud 800 and the shunt 200 through the transfer device lumen 26 into the handle 10/10’ of the delivery catheter 304. Prior to clinical use (e.g., during device shipping), the Touhy-borst adapter 21 is closed around the delivery wire 308 to prevent inadvertent movement or dislodgement of the shunt 200 and the shroud 800 from transfer device 20/20’. Further, the Touhy-borst adapter 21 is configured to prevent backflow of fluid during clinical use of the device. The Luer fitting 22 of the transfer device 20 is configured to be engaged and be coupled to the Luer fitting 12 of the handle 10/10’, as shown in FIGS. 16A-16B. The stopcock 25 is a 3-way valve configured to stop fluid flow when the stopcock 25 is disposed in a closed position. The housing 24 and the transfer device lumen 26 are configured to have the shroud 800 and shunt 200 disposed within, as shown in FIGS. 15C-D, and the detailed views of the housing 24 in FIGS. 15C and 15E-F. When the shroud 800 and shunt 200 are disposed within the transfer device lumen 26, the shroud 800 is disposed over the shunt 200. The anchoring mechanism 229 of the shunt 200 is in the compressed configuration within the housing 24 as shown in the detail views of FIGS. 15C, 15F; a passive seal 23 of the housing 24 holds the anchoring mechanism 229 in the compressed configuration (FIGS. 15C, 15F) for delivery assembly 300 handling, preparation and flushing of shunt 200.

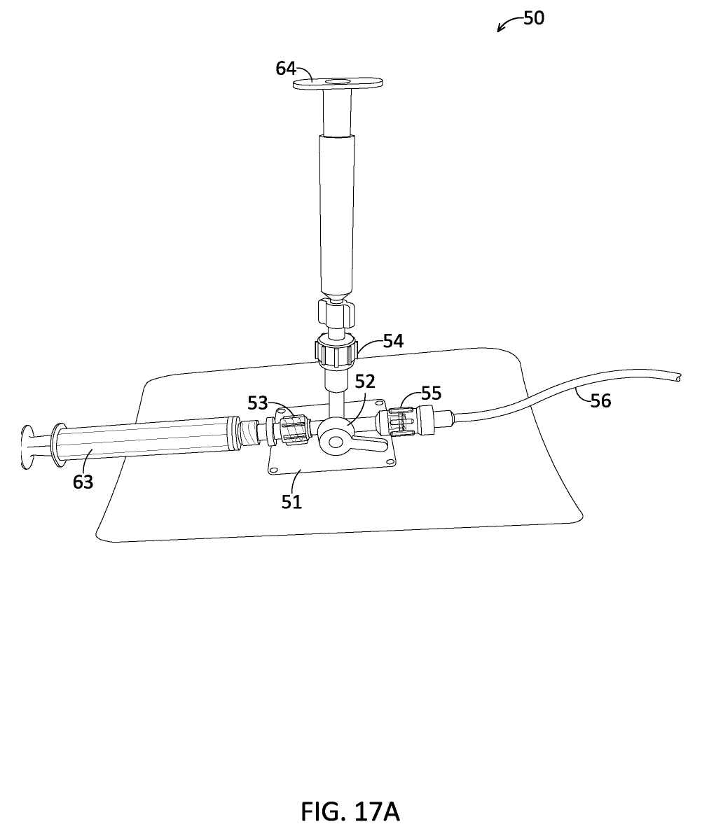

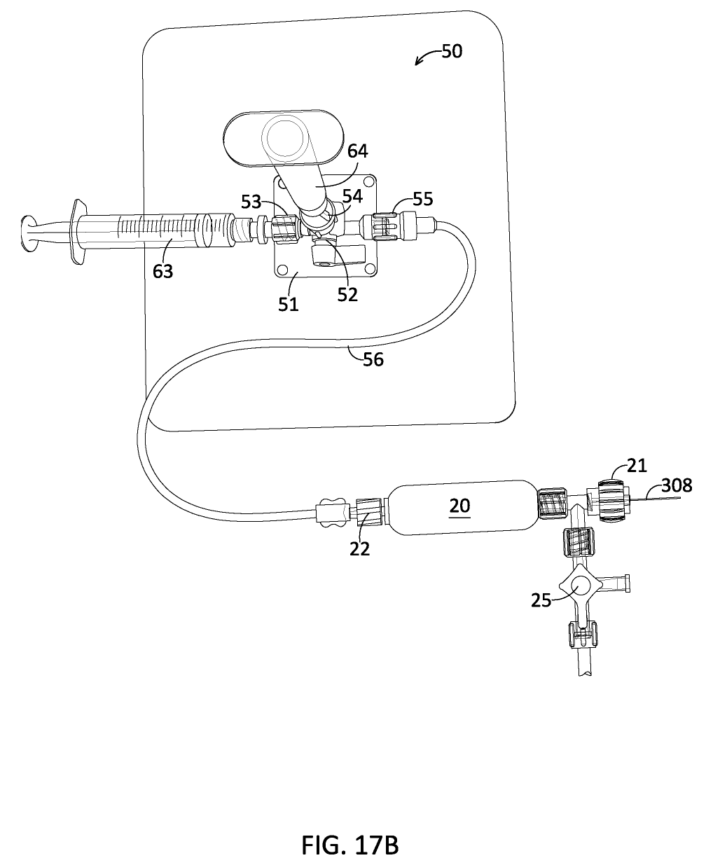

With the shunt 200 disposed within the shroud 800, the shunt 200 and shroud 800 are loaded into the transfer device 20/20’. As shown in FIGS. 15C and 15F, the distal portion 204 of the shunt 200 and the distal portion 830 of the shroud 800 are disposed partially within the passive seal lumen 23’, within the fluid path of the transfer device lumen 26. The passive seal 23 is configured to seal around a portion of the distal anchoring mechanism 229 and the distal portion 204 of the shunt 200 (FIG. 15F) to facilitate fluid flushing through the shunt lumen 207 and fluid flow from reservoir 50 (described below in FIGS. 17A-B) while confirming patency of the shunt valve 209 while preparing the shunt 200 for implantation. The passive seal lumen 23’ provides an interference fit with the distal anchoring mechanism 229 and the distal portion 204 of shunt 200 such that fluid entering the transfer device lumen 26 via Luer fitting 22 is directed into the shunt lumen 207 and minimizes fluid flow around the exterior surfaces of the distal anchoring mechanism 229 and the shunt body 203.

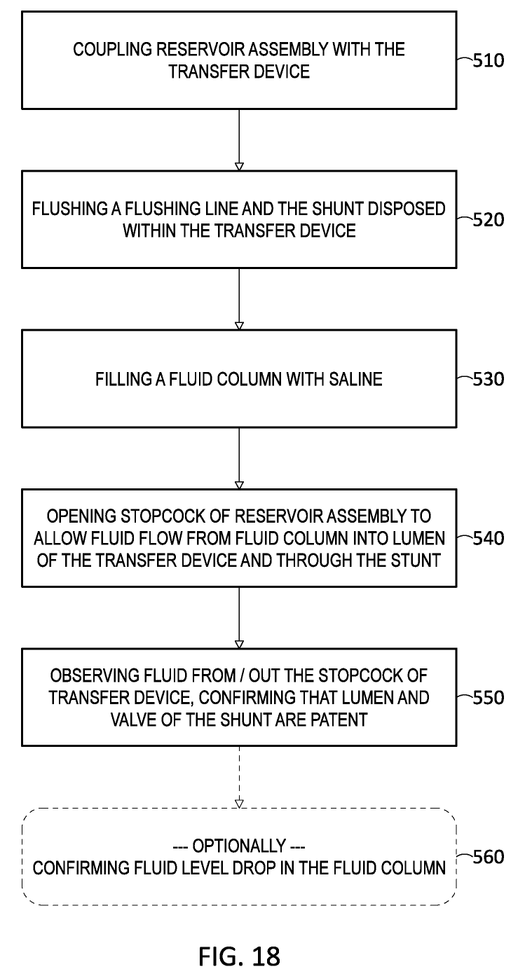

The clinician may complete a patency test 500 (FIG. 18) when the shroud 800 and shunt 200 are disposed within the transfer device 20/20’, then, confirm flow out of the stopcock 25 (e.g., prior to implantation of the shunt 200), as further described in FIGS. 17A-B.

Following valve patency test 500, the clinician couples the transfer device 20 to the handle 10/10’ (FIGS. 16A-B) and distally translates (i.e., pushes) the shroud 800 and the shunt 200 using the delivery wire 308 from the passive seal 23 into and through lumen 26 (FIGS. 15BG and 16A-B) of the transfer device 20/20’ into the handle 10/10’, and further into the lumen 305 of the delivery catheter 304. As previously disclosed, the shroud 800 translates the shunt 200 through the handle 10/10’ and into the delivery catheter lumen 305 through the shroud distal radiopaque marker 862 pushing against the shunt engagement region 244 and the proximal retention feature 850 pushing against the shunt proximal portion 202. The transfer device lumen 26 is in fluid communication with the handle 10/10’. The clinician may use the markers of the shroud 800 and/or the delivery wire 308 to assist with the positioning of the shroud 800 and the shunt 200 within the handle 10/10’, so that the delivery assembly 300 is suitable for the medical procedure. The clinician can continue advancing the shroud 800 and the shunt 200 into a distal portion of delivery catheter first lumen 305 via delivery wire 308. One or more markings on a proximal portion of the delivery wire 308 are configured to indicate when the delivery wire 308 has been sufficiently advanced, such that the shunt 200 and the shroud 800 are advanced into a distal portion of lumen 305, which provides additional column strength and resistance to kinking in the distal portion 344 of delivery catheter 304 during the implantation procedure.

FIG. 17 A illustrates a perspective view of a reservoir assembly 50, according to embodiments of the disclosed inventions. FIG. 17B illustrates a top view of the reservoir assembly 50 and a perspective view of the transfer device 20/20’, according to embodiments of the disclosed inventions. As shown in FIGS. 17A-B, the reservoir assembly 50 comprises a 3-way stopcock 52 having a plurality of Luer fittings 53, 54 and 55; the stopcock 52 is secured into a base 51. Base 51 includes a hinged or swivel mechanism coupled to stopcock 52; reservoir assembly 50 is packaged with fluid column 64 lying parallel to the die card surface of FIG. 17; when a clinician or technician is ready to prepare shunt 200 for clinical use, the reservoir assembly 90 is rotated 90 degrees upward from the die card until the base 51 provides a positive lock to hold reservoir assembly 50 and fluid column 64 upright as shown in FIG. 17A; the positive lock is achieved by an interference fit of the molded hinge components in base 51 (not shown).

As shown in FIG. 17B, the reservoir assembly 50 is in fluid communication with the Luer fitting 22 and lumen 26 of the transfer device 20, via a flushing line 56 of the reservoir assembly 50. The Luer fitting 53 of the reservoir assembly 50 is coupled to a syringe 63 to allow fluid flushing through the stopcock 52 and Luer fitting 55, into the flushing line 56 connected to the transfer device 20 into the transfer device lumen 26. The stopcock 52 can open to allow fluid flow from the syringe 63 for flushing of the flushing line 56 connected to the transfer device 20. The fluid flushing from the reservoir assembly 50 into the transfer device 20/20’ is configured to flush fluid through shunt lumen 207 via the passive seal 23 to remove air bubbles that can prevent, decrease fluid flow or increase backflow through valve 209 of the shunt 200 (not shown). Additionally, the Luer fitting 54 of the reservoir assembly 50 is coupled to a fluid column 64. The stopcock 52 can open to fill the column 64 with fluid (e.g., saline, heparinized saline, sterile water); and the stopcock 52 can open to allow fluid to flow from the fluid column 64 into transfer device 20 via the flushing line 56. With the fluid column 64 filled and the stopcock 52 open to the transfer device 20/20’, fluid is allowed to flow from the column 64 through the flushing line 56 into transfer device 20/20’ and the transfer device lumen 26. The passive seal 23 of the housing 24 of the transfer device 20/20’ holds the shunt anchoring mechanism 229 in the compressed configuration and provides a fluid seal around the shunt 200 disposed in the lumen 26 of the transfer device 20/20’ (FIGS. 15C and 15F-G). The passive seal 23 directs fluid flow received from the flushing line 56 through the lumen 26 into the distal portion of shunt lumen 207, out of shunt valve 209, the transfer device lumen 26 and out of the stopcock 25 of the transfer device 20. Fluid column 64 can be filled to a height known to provide a desired or known amount of fluid pressure on valve 209 (via flushing line 56, transfer device lumen 26 and passive seal lumen 23’).

FIG. 18 illustrates a patency test 500 for confirming patency of the shunt 200, according to the embodiments of the inventions. The patency test 500 provides confirmation that the shunt 200 and the shunt valve 209 will provide a therapeutic flow rate of CSF (e.g., more than 5 mL/hr.) from the SAS to the venous system at a target differential pressure range between the SAS and venous system after the shunt deployment procedure. The transfer device 20/20’ (FIGS. 15A-G and 16A-B) and reservoir assembly 50 (FIGS. 17A-B) are configured to be used in the manner, actions, and/or steps described in the patency test 500 of FIG. 18. The patency test 500 comprises: coupling 510 the reservoir assembly 50 with the transfer device 20/20’ through the flushing line 56; flushing 520 (e.g., at least one time or preferably two or more times), the flushing line 56 and the shunt 200 disposed within the transfer device 20 with saline or heparinized saline via the syringe 63; filling 530 the fluid column 64 with saline to a height that correlates with an expected differential pressure between the SAS and venous system; opening 540 the stopcock 52 to allow fluid flow from the fluid column 64 to the flushing line 56 into the lumen 26 of the transfer device 20 and through the shunt 200 (e.g., into CSF lumen 207 and out valve 209); observing 550 fluid flow from/out of 3-way stopcock 25 of transfer device 20/20’, confirming that lumen 207 and valve 209 of the shunt 200 are patent. Optionally, confirming 560 that the fluid level in the fluid column 64 drops a predetermined distance (e.g., 1 cm) with a specified time period (e.g., five minutes) during which fluid flows from the fluid column 64 into the flushing line 56 and transfer device 20/20’ and out of shunt 200. The predetermined fluid drop in fluid column 64 from a specified fluid height in fluid column 64 over a specified time period can be correlated to known fluid flow rates for a range of fluid pressures applied through shunt lumen 207 and valve 209 to provide an indicator of therapeutic performance and anticipated CSF flow rates through shunt 200 during clinical use of the device.

Using the transfer device 20/20’ and reservoir assembly 50, for example, as described in the patency test 500, eliminates the prior art need for the clinician to advance or proximally withdraw the shunt 200 into delivery catheter 304 through the penetrating element 350; avoiding the risk of the penetrating element 350 skiving, tearing, or damaging the shunt 200 including the shunt lumen 207 and the shunt valve 209 during loading. Further, the use of the transfer device 20/20’ and reservoir assembly 50 eliminates the prior art need for a clinician to directly handle the shunt 200 during device preparation for clinical use and the implantation procedure; the direct handing of the shunt 200 or conventional shunt devices implanted through open surgical procedures increases the risk of compromising the device sterility and introducing contaminants onto the shunt 200 before the deployment procedure which can cause patient infection. Additionally, the transfer device 20/20’ eliminates the need to directly handle the shunt 200 through all of the preparation for clinical use steps such as flushing, patency test 500, and delivery catheter loading.

Further, it should be appreciated that the transfer device 20/20’ lumen 26 and the first lumen 305 of the delivery catheter 304 should have substantially similar diameters, such that there is no need for the shroud 800 to accommodate stretching the shunt 200 and necking down the outer diameter of shunt 200 while loading the shunt 200 into delivery catheter 304. Stretching the shunt 200 creates the potential for damaging the polymer shunt body and/or biocompatible coating on the exterior surface of the shunt 200 and/or valve 209. Stretching of the shunt 200 was needed in prior art systems, which requires a complex shroud design to maintain control over the shunt 200 within delivery catheter first lumen 305 and significantly increases the friction between shunt 200 and first lumen 305 during the implantation procedure. In addition to the complex shroud design, the prior art systems relied on a complex apparatus for flushing shunt 200, stretching shunt 200, and loading shunt 200 through delivery catheter penetrating element 350 into delivery catheter first lumen 305, for example, as disclosed in U.S. Patent Application Publication No. US20210228846. Further, the prior art need for stretching of the shunt 200 required the clinician to overcome the resistance between the shunt 200 and first lumen 305 of the delivery catheter 304, while advancing the shunt 200 through first lumen 305 and out of the penetrating element 305 during the shunt deployment procedure. The act of overcoming said resistance can reduce important tactile feedback received by the clinician during the shunt deployment procedure. Embodiments of the delivery catheter 304, shroud 800 and transfer device 20 disclosed herein overcome the drawbacks stemming from shunt stretching, decrease resistance to shunt advancement, and provide an overall smoother and more controlled deployment of shunt 200.

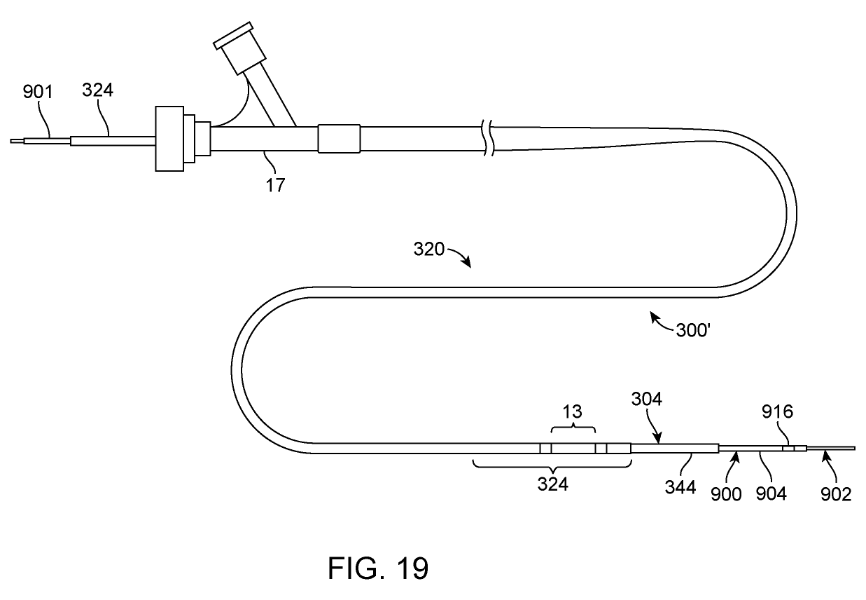

Referring now to FIG. 19, another delivery assembly 300’ for facilitating a shunt delivery procedure will be described. The delivery assembly 300’ is similar to the delivery assembly 300 illustrated in FIG. 1, with the exception that, instead of an anchor 700, the delivery assembly 300’ comprises one or more elongate navigation members (and in this case, a microcatheter 900 and a guidewire 902) to facilitate navigation of the delivery catheter 304. Thus, the delivery assembly 300’ comprises the guide catheter 320, delivery catheter 304, microcatheter 900, and guidewire 902.

The microcatheter 900 can have an outer diameter smaller than the inner diameter of the lumen 305 (shown in FIG. 2B) of the delivery catheter 304, such that microcatheter 900 can be introduced into lumen 305 via the Luer fitting 12 on handle 10 (shown in FIG. 2A) of the delivery catheter 304, advanced through the lumen 305 and guard member lumen 405 (shown in FIG. 2B), and further advanced out of the distal portion 344 of delivery catheter 304. For example, the microcatheter 900 can be an 0.017” microcatheter (e.g., Scientia Plato 17, Medtronic Phenom 17, Wallaby PNovus 17, Terumo Headway Duo 156, etc.) or intravascular catheter advancement device (e.g., as disclosed in US11224450B2, which is expressly incorporated herein by reference in its entirety). The microcatheter 900 comprises a proximal end 901 having a hub (not shown) a distal end 904, and a lumen (not shown) extending between an opening in each of the proximal end 901 and distal end 904 of the microcatheter 900. The microcatheter 900 further comprises one or more radiopaque markers 916 disposed on the distal end 904 of the microcatheter 900. The guidewire 902 may be similar to the guidewire 302 described above, but has an outer diameter smaller than the inner diameter of lumen of microcatheter 900 (e.g., a 0.014” guidewire), which provides for introduction of a distal end 914 of the guidewire 902 into the hub of the microcatheter 900, through the lumen, and out of the distal end 904 of the microcatheter 900.

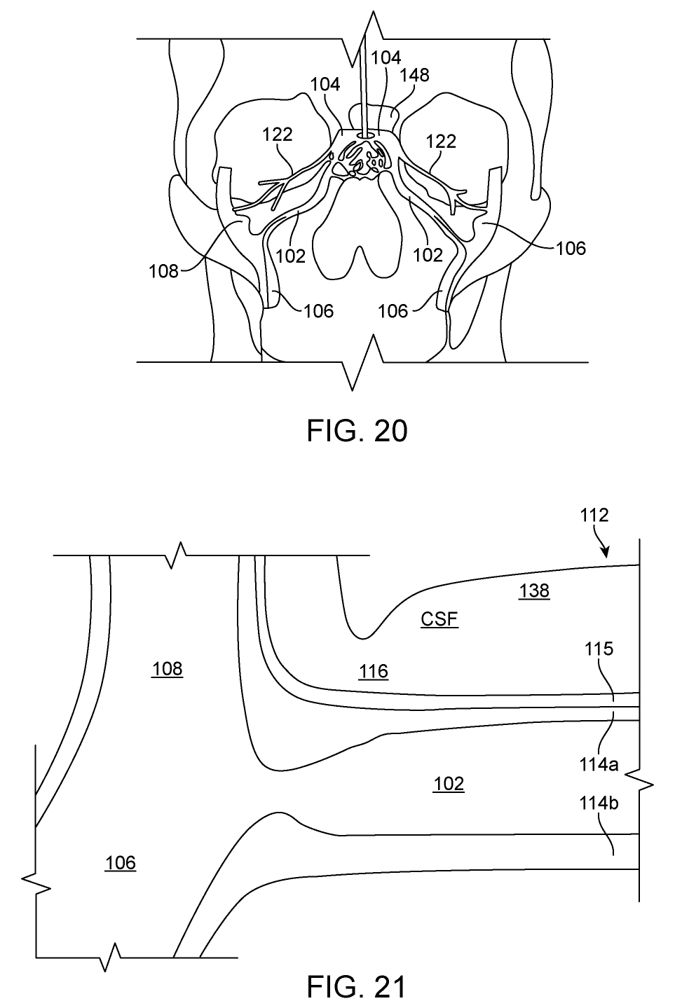

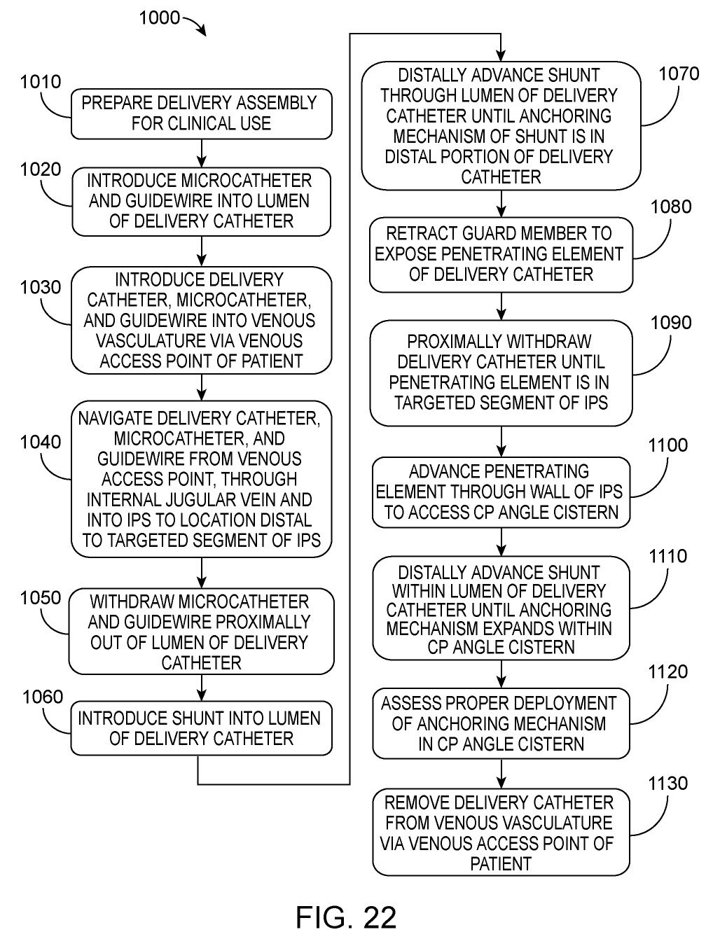

Referring to FIGS. 20-21, the delivery assembly 300’ may be used to access the subarachnoid space and deliver the endovascular CSF shunt 200 into the subarachnoid space (SAS) 116 of a patient. With each side of the patient’s head, an inferior petrosal sinus (IPS) 102 connects a cavernous sinus (CS) 104 to a jugular vein 106 and/or a jugular bulb 108. The IPS 102 is a relatively small diameter intracranial venous sinus that facilitates drainage of cerebral venous blood into the jugular veins 106. In some patients, the junction of the IPS 102 and the jugular vein 106 occurs within the jugular bulb 108. However, in other patients, this junction can occur at other locations in the jugular vein 106. Moreover, while the IPS 102 is illustrated in FIGS. 20 and 21 as a single sinus passageway, in some patients, the IPS 102 can be a plexus of separate channels that connect the CS 104 to the jugular vein 106 (not shown) and/or the jugular bulb 108. A superior petrosal sinus (SPS) 122 also connects the CS 104 to a sigmoid sinus (distally located to jugular bulb 108).

The IPS 102 is formed by a cylindrical layer of dura mater 114, which creates a hollow lumen through which blood flows. A cross-section of the IPS 102 orthogonal to the plane depicted in FIG. 21 would show that the cylindrical layer of dura mater 114 forming the IPS 102 is surrounded by bone for about 270° of its circumference with the remaining portion of the circumference of the IPS 102 (i.e., the IPS wall 114) covered by a delicate and avascular layer of arachnoid matter 115 (also referred to herein as the arachnoid layer). The IPS wall 114 may be topologically divided between an upper IPS wall 114a outside of which a portion of the SAS 116, and in particular, the cerebellopontine (CP) angle cistern 138, resides, and an lower IPS wall 114b that sits in a channel in the clivus bone (not shown). A suprasellar cistern 148 exists above the upper portion or roof of the CS 104. The CP angle cistern 138 and suprasellar cistern 148 are both filled with cerebrospinal fluid (CSF).

As will be described in further detail below, the delivery assembly 300’ can be used for deploying the shunt 200 within the CP angle cistern 138 of the patient. In particular, the delivery assembly 300’ can be employed to penetrate the IPS wall 114 and the arachnoid layer 115 to access the CP angle cistern 138 from within the IPS 102 for delivery of the shunt 200 at the target implantation site within the anastomosis between the IPS 102 and the CP angle cistern 138.

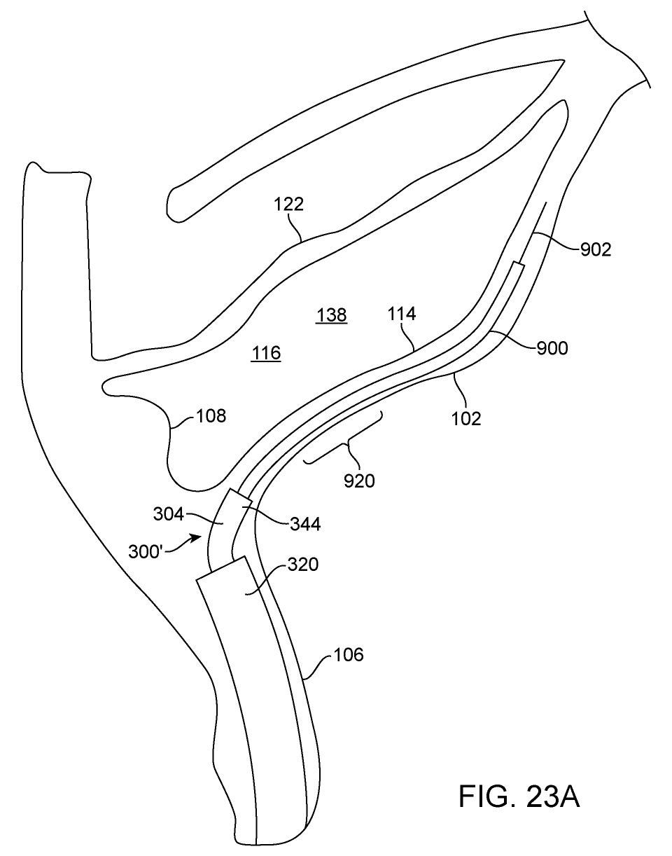

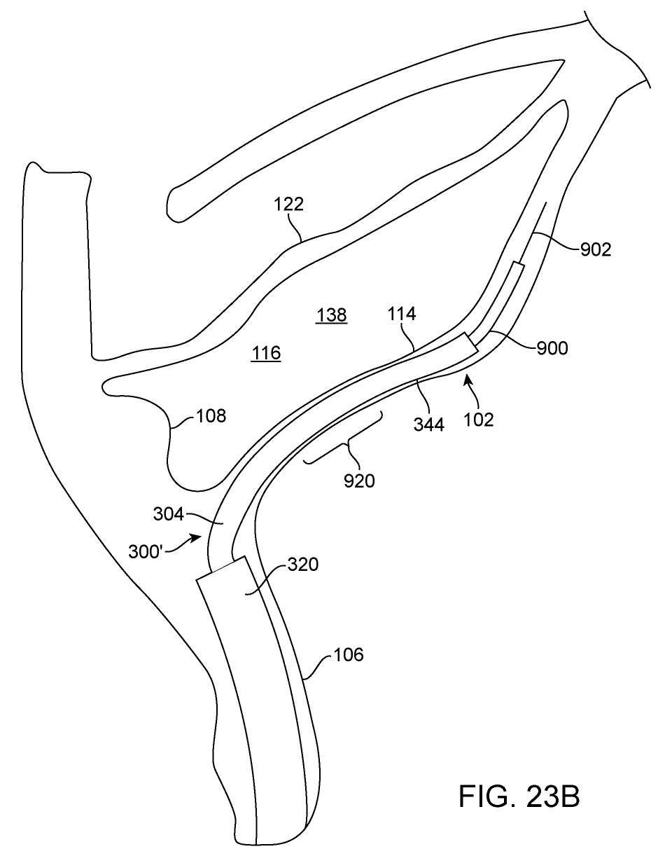

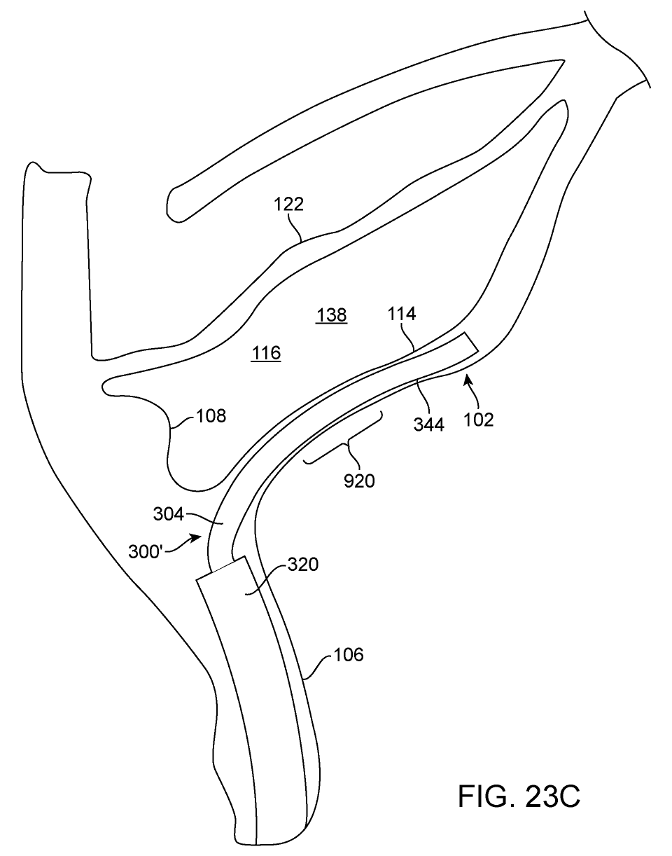

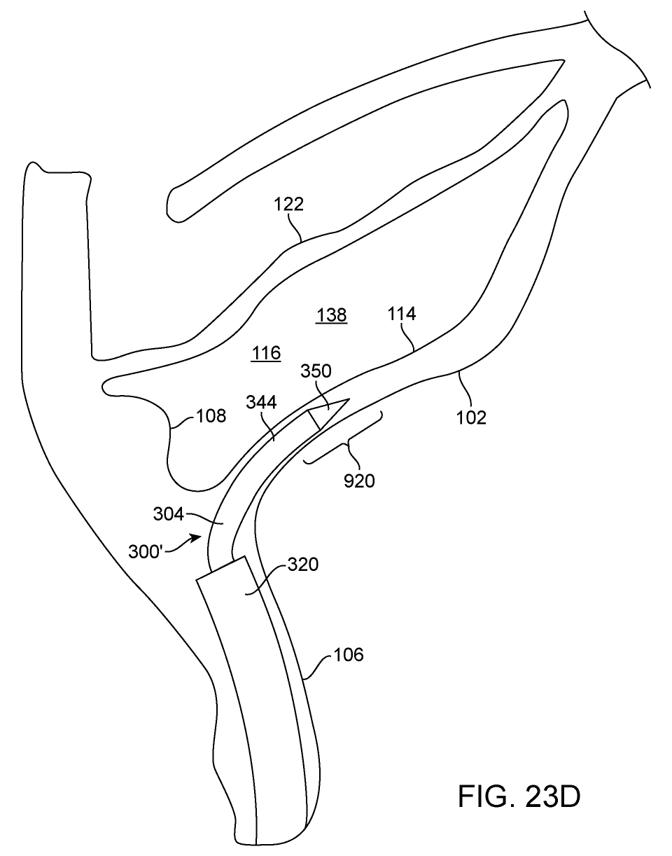

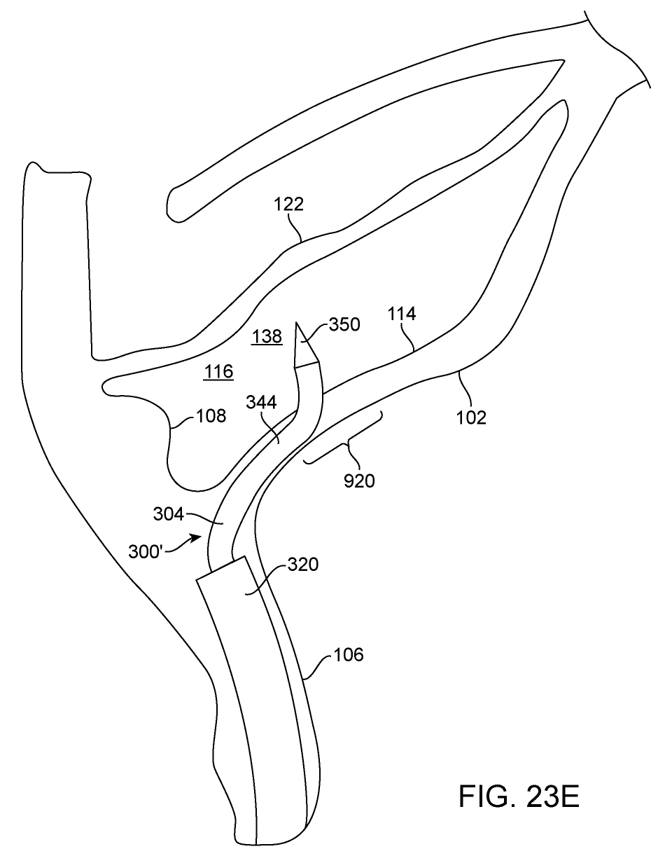

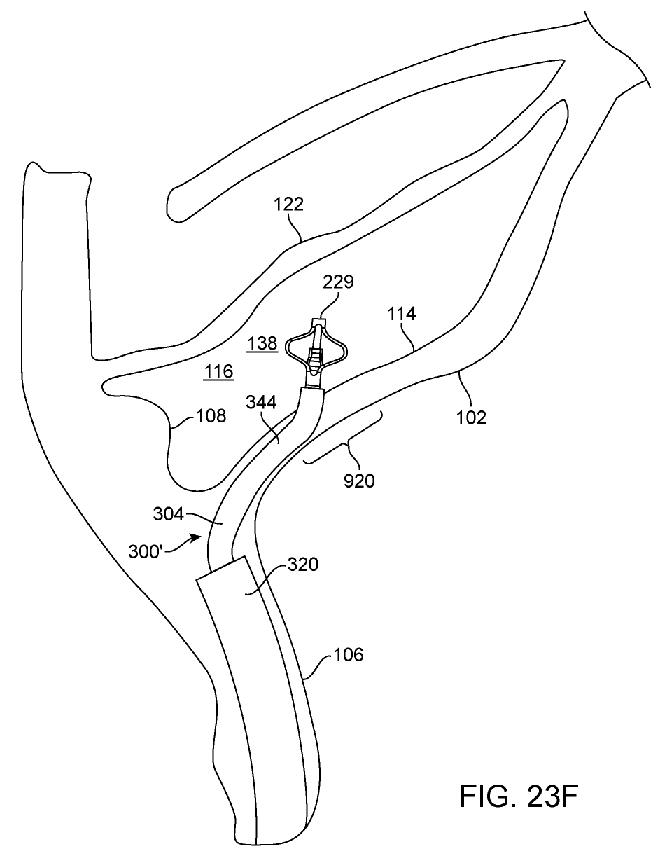

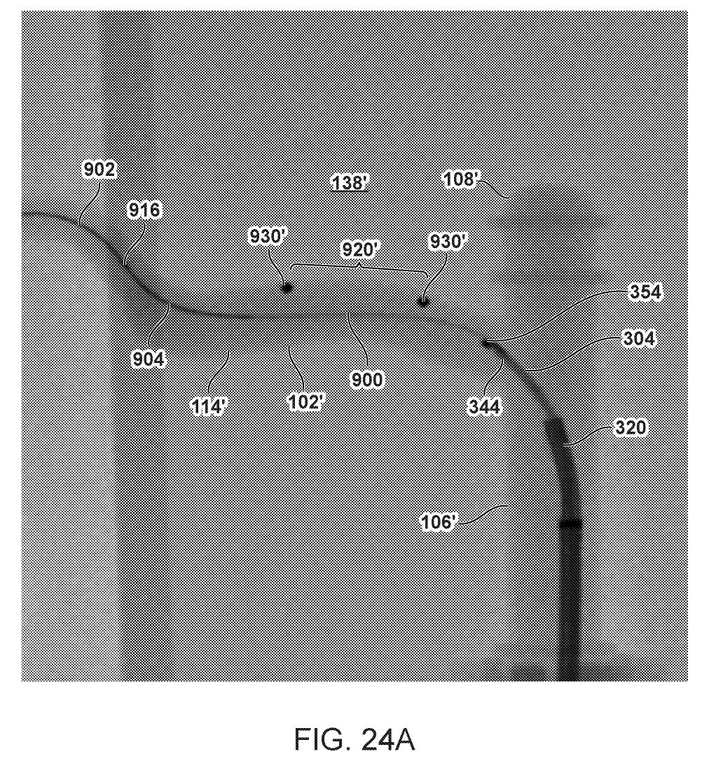

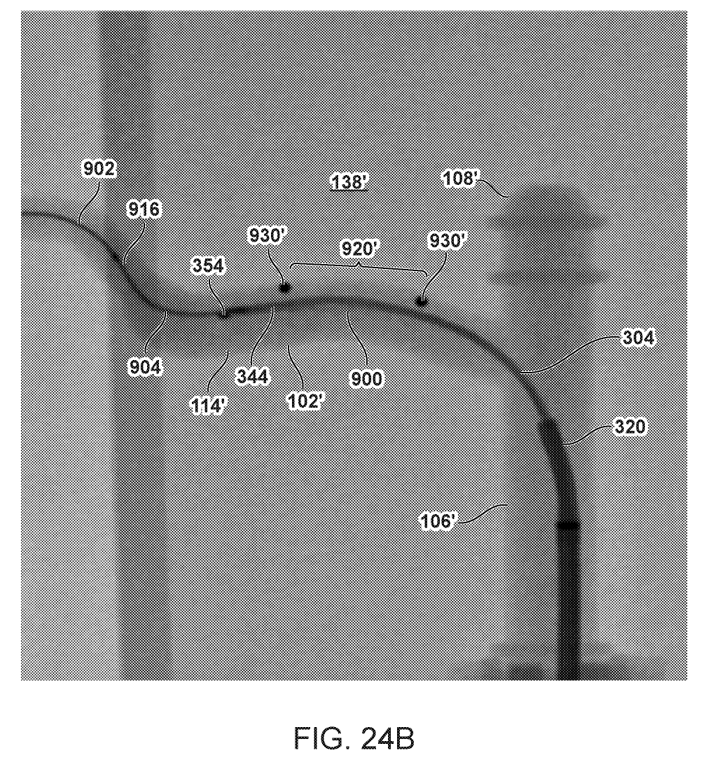

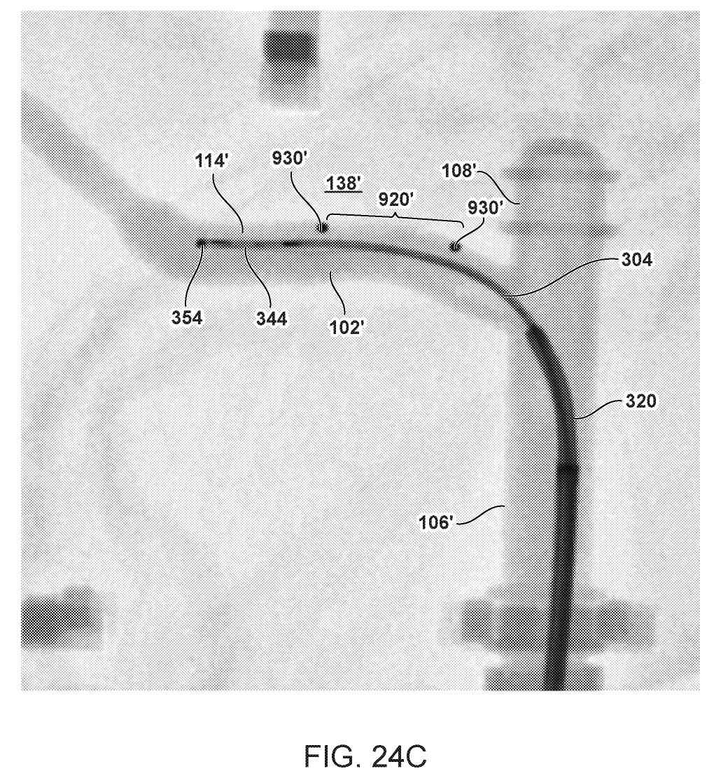

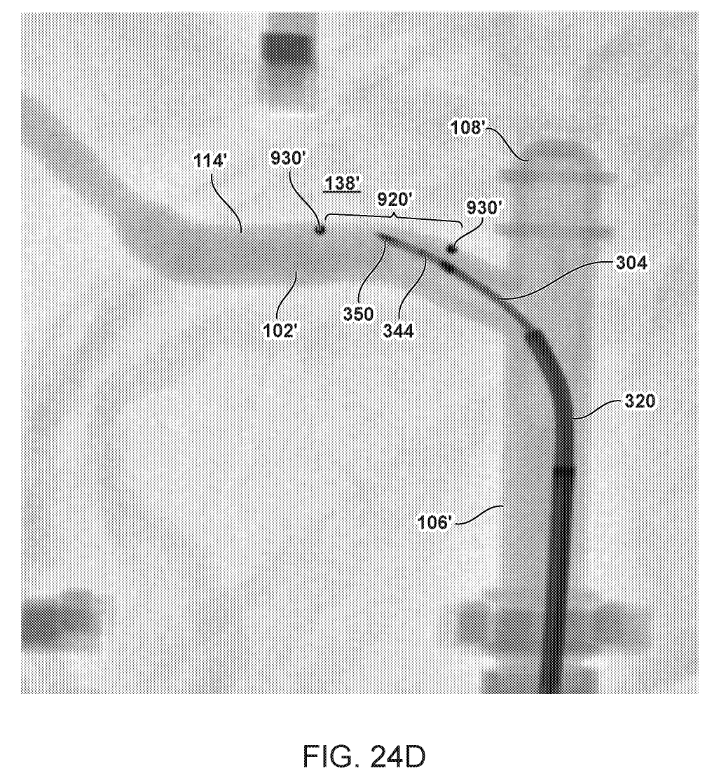

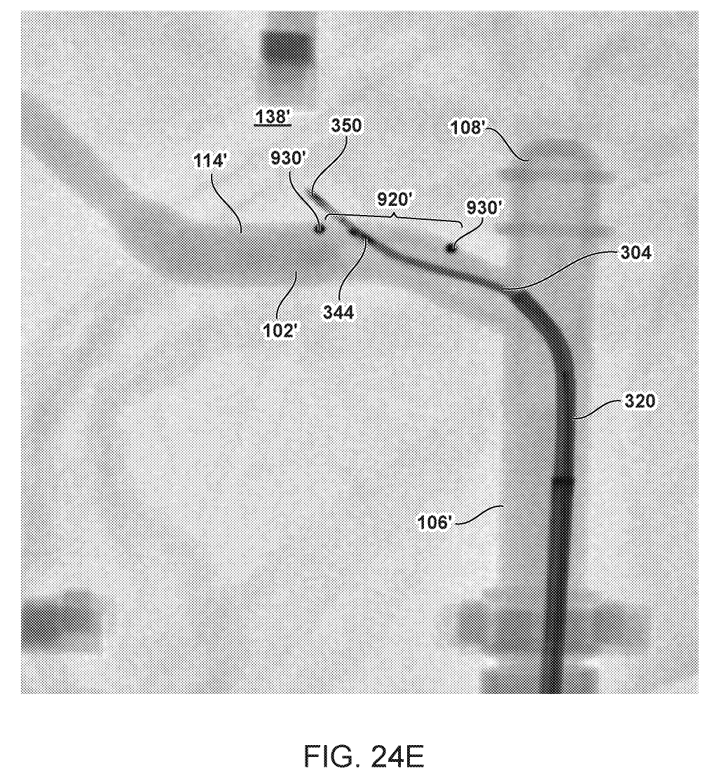

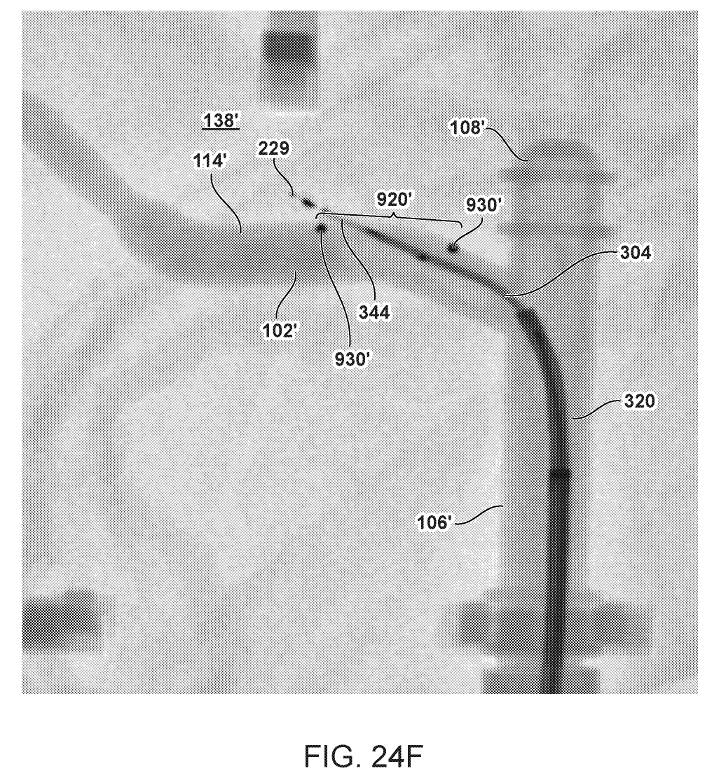

Referring now to FIGS. 22 and 23A-23F, one shunt deployment procedure 1000 of operating the delivery assembly 300’ to endovascularly deploy a shunt 200 within the SAS 116 will be described. In the illustrated embodiment, the shunt deployment procedure 1000 is described with respect to an exemplary target penetration site in the IPS 102 to access the CSF-filled CP angle cistern 138 with the delivery catheter 304 and deploying the shunt 200 within an anastomosis between the IPS 102 and CP angle cistern 138. In particular, the delivery assembly 300’ disclosed herein can be used to penetrate, for example, the IPS wall 114 and the arachnoid layer 115 (shown in FIG. 21) to access the CP angle cistern 138 from within the IPS 102 for endovascular delivery of the shunt 200 within the anastomosis between the IPS 102 and the CP angle cistern 138. The shunt deployment procedure 1000 is also illustrated in FIGS. 24A-24F through x-ray images of a simulated procedure completed within an in vitro anatomic model that includes features representative of the jugular vein 106’, jugular bulb 108’, IPS 102’, IPS wall 114’, and CP angle cistern 138’.

It should be appreciated, however, that the delivery assembly 300’ can be used to access any region of the SAS 116 from target penetration sites located in other dural venous sinuses (e.g., CS 104 and SPS 122, sagittal sinus, transverse sinus, sigmoid sinus, etc. not shown), and are not limited exclusively to IPS 102. For example, the cerebrospinal fluid (CSF)-filled CP angle cistern 138 can be accessed from a target penetration site in the SPS 122 for implantation of shunt 200 at the target implantation site within an anastomosis between the SPS 122 and the CP angle cistern 138, or the CSF-filled suprasellar cistern 148 (shown in FIG. 20) can be accessed from a target penetration site in the upper portion or roof of the CS 104 (via the IPS 102) for implantation of shunt 200 at a target implantation site within an anastomosis between the CS 104 and the suprasellar cistern 148.

Initially, the delivery assembly 300’ is prepared for clinical use, for example, by hydrating and/or flushing with saline or heparinized saline the guide catheter 320, delivery catheter 304, microcatheter 900, and guidewire 902 (step 1010). The microcatheter 900 and the guidewire 902 are then introduced into the lumen 305 of the delivery catheter 304 (step 1020). For example, the guidewire 902 may be introduced and advanced through the lumen of the microcatheter 900, and the microcatheter 900 and guidewire 902 may then be introduced into the Luer fitting 12 on the handle 10 (see FIG. 13) of the delivery catheter 304, such that the delivery catheter 304, microcatheter 900, and guidewire 902 are in a coaxial arrangement, and then advanced distally within the lumen 305 until their respective distal ends 904 and 914 of the microcatheter 900 and guidewire 902 are disposed at the distal portion 344 of the delivery catheter 304 or distal to the distal opening of the lumen 405 of the guard member 400 (shown in FIG. 2B).

The coaxial arrangement of the delivery catheter 304, microcatheter 900, and guidewire 902 are then introduced into the patient’s venous vasculature via venous access point (e.g., a femoral vein, subclavian vein, or internal jugular vein access point) in the patient created using the Seldinger technique (step 1030). In a preferred method, the delivery catheter 304, microcatheter 900, and guidewire 902 may be simultaneously introduced into the patient’s venous vasculature. In an alternative method, the delivery catheter 304, microcatheter 900, and guidewire 902 may be sequentially introduced into the patient’s venous vasculature. For example, the delivery catheter 304, then the microcatheter 900, and then the guidewire 902 may be introduced into the patient’s venous vasculature. In optional methods, an initially introduced microcatheter or guidewire may be swapped out for a new or different microcatheter or guidewire. In another optional method, the guide catheter 320 can first be introduced into the venous vasculature until a distal end portion of the guide catheter 320 is disposed within internal jugular vein 106, and then the coaxial arrangement of the delivery catheter 304, microcatheter 900, and guidewire 902 can be inserted through the guide catheter 320 into the venous vasculature of the patient. It should be appreciated that other venous access points (e.g., subclavian vein) in the patient can be used to navigate the coaxial arrangement of the delivery catheter 304, microcatheter 900, and guidewire 902 to the internal jugular vein 106. The coaxial arrangement of the delivery catheter 304, microcatheter 900, and guidewire 902 is then navigated from the venous access point in the patient through the internal jugular vein 106 and into the IPS 102 of the patient to a location distal to a targeted IPS segment 920 (step 1040). For example, while the distal portion 344 of the delivery catheter 304 is located proximal to the targeted IPS segment 920 in the IPS 102, the microcatheter 900 and guidewire 902 can be distally advanced within the IPS 102 until the respective distal ends of the microcatheter 900 and guidewire 902 are distal to the targeted IPS segment 920 in the IPS 102 (see FIGS. 23A and 24A). Notably, as shown in FIG. 24A, the targeted IPS segment 920’ is located between two radiopaque markers 930’ embedded in the IPS wall 114’ of the in vitro model. Visualization of the radiopaque marker 916 located on the distal end 904 of the microcatheter 900 facilitates location of the distal end of the microcatheter 900 at a location distal to the targeted IPS segment 920 of the IPS 102, while visualization of the radiopaque marker 354 located on the distal portion 344 of the delivery catheter 304 (and in particular, on the penetrating element 350) facilitates location of the distal portion 344 of the delivery catheter 304. The delivery catheter 304 may then be distally advanced over microcatheter 900 until the distal portion 344 is distal to the targeted IPS segment 920 in the IPS 102 (see FIGS. 23B and 24B).

The microcatheter 900 and guidewire 902 are then withdrawn proximally out of the lumen 305 of the delivery catheter 304 while maintaining the distal portion 344 of the delivery catheter 304 at a location within IPS 102 distal to the targeted IPS segment 920 (step 1050) (see FIGS. 23C and 24C). If a guide catheter 320 is used in the procedure, a rotating hemostasis valve (not shown) on the Luer assembly 17 can be tightened down around the proximal portion 324 of delivery catheter 304 to secure the position of the delivery catheter 304 for the next step of the shunt deployment procedure 1000.

Next, the shunt 200 is introduced into the lumen 305 of the delivery catheter 304 (step 1060). In particular, the transfer device 20 may be coupled to the handle 10 of the delivery catheter 304. The transfer device 20 may be coupled to the handle 10 of the delivery catheter 304 via the Luer fitting 22 of the transfer device 20 and Luer fitting 12 of the delivery catheter handle 10 (see FIGS. 16A-16B). The shunt 200 may be associated with a shroud assembly comprising the shroud 800 and the delivery wire 830 connected to the shroud 800, while the shunt 200 may be disposed within the lumen 833 of the shroud 800 (e.g., as detailed above in connection with FIGS. 7-9). The shroud assembly, with the shunt 200 disposed within the lumen 833 of the shroud 800, may be disposed within the lumen 26 of the transfer device 20 (see FIGS. 15A-15D). The shroud assembly, while the shunt 200 is disposed within the lumen 833 of the shroud 800, may then be distally advanced from the lumen 26 of the transfer device 20 into the lumen 305 of the delivery catheter 304. Optionally, the shunt lumen 207 may be flushed and valve patency may be performed for the shunt 200 in the manner described above with respect to FIG. 18. The shunt 200 may be advanced from the transfer device 20 into the lumen 305 of delivery catheter 304 by distal advancement of delivery wire 830 and shroud 800.

Then, the shunt 200 is distally advanced through the lumen 305 of the delivery catheter 304 until the anchoring mechanism 229 (shown in FIGS. 7A-7D) of the shunt 200 is at the distal portion 324 of the delivery catheter 304 (step 1070). Next, the guard member 400 (see FIG. 2B) is retracted to expose the penetrating element 350 of the delivery catheter 304 (step 1080). The guard member 400 may be retracted using the tether actuation mechanism 18 as described in connection with FIG. 14. Next, the delivery catheter 304 is proximally withdrawn in the IPS 102 until the penetrating element 350 is within targeted IPS segment 920 (step 1090) (see FIGS. 23D and 24D). The orientation of the penetrating element 350 relative to the targeted IPS segment 920 can be adjusted through manipulation of the handle 10. The penetrating element marker 354 can be used to visualize the orientation of penetrating element 350 at the target penetration site in the IPS wall 114.

It should be appreciated that distally advancing the distal portion 344 of the delivery catheter 304 distal to the targeted IPS segment 920, retracting the guard member 400 to expose the penetrating element 350, and proximally withdrawing the delivery catheter 304 to the targeted IPS segment 920 may have several benefits. For example, advancing the distal portion 344 of the delivery catheter 304 distal to the targeted IPS segment 920 within the IPS 102 prior to retracting the guard member 400 to expose the penetrating element 350 facilitates the placement of distal portion 344 of the delivery catheter 304 in a more mechanically neutral state. That is, due to the curvature between the jugular vein 106 and the IPS 102, the distal portion 344 of the delivery catheter 304 positioned directly at the targeted IPS segment 920 may retain stored bending or compressive forces (spring forces) that can bias the distal portion 344, such that it may cause unintended motion of the penetrating element 350 upon retraction of the guard member 400. By overshooting the distal portion 344 of the delivery catheter 304 into a more distal, straighter, segment of the IPS 102, these spring forces are reduced, allowing the penetrating element 350 to be exposed in a lower-risk, more controlled environment. Subsequent proximal withdrawal of the delivery catheter back to the targeted IPS segment 920 further improves mechanical stability by relieving forward axial loading caused by the curvature between the jugular vein 106 and the IPS 102 and allowing the delivery catheter 304 to settle into better alignment with the IPS wall 144. This pullback positions the system in a more favorable force regime, reducing the likelihood of forward “kick” and enabling more controlled and predictable engagement of the penetrating element 350 with the IPS wall 114, thereby facilitating safer and more precise penetration.

Although the foregoing overshoot-and-pullback sequence may be preferable in many implementations, it is not required in all cases. In certain anatomies, for example, where limited space exists distal to the targeted IPS segment 920 within the IPS 102 or where distal advancement is otherwise constrained, the distal portion 344 of the delivery catheter 304 can instead be positioned directly at the targeted IPS segment 920 within the IPS 102 and the guard member 400 retracted to expose the penetrating element 350 at the targeted IPS segment 920. In such cases, the procedure may be performed without distal overshoot, provided that appropriate control of position and orientation of the distal portion 344 of the delivery catheter 304 is maintained.

Next, the penetrating element 350 is advanced through IPS wall 114 to access CP angle cistern 138 of SAS 116 (step 1100). For example, the delivery catheter 304, while angled toward the IPS wall 114, may be advanced, such that the penetrating element 350 pierces and then traverses through the through IPS wall 114 (and arachnoid layer 115) into the CP angle cistern 138 (see FIGS. 23E and 24E). Next, the shunt 200 is advanced distally in the lumen 305 of the delivery catheter 304 until the anchoring mechanism 229 expands within CP angle cistern 138 (step 1110) (see FIGS. 23F and 24F). For example, the delivery wire 830 and shroud 800, with the shunt 200 disposed within the shroud lumen 833 (e.g., as detailed above in connection with FIGS. 7-9), can be distally advanced within the lumen 305 of the delivery catheter 304, such that the anchoring mechanism 229 exits the lumen 305 of the delivery catheter 304 into the CP angle cistern 138. Once the anchoring mechanism 229 is no longer constrained by the delivery catheter 304, the anchoring mechanism 229 transitions from its compressed delivery configuration to its expanded deployed configuration.

Next, proper deployment of the anchoring mechanism 229 of the shunt 200 may be assessed (step 1120). In one embodiment, this assessment may be performed using imaging, such as fluoroscopic imaging, to confirm appropriate positioning and expansion of the anchoring mechanism 229 relative to the target anatomy, including the CP angle cistern 138 and adjacent venous structures. In addition to, or as an alternative to, imaging-based confirmation, a mechanical assessment may be performed by applying a proximal force to the delivery catheter 304 (e.g., a slight “tug” or pullback). Because the proximal retention feature 850 of the shroud 800 (see FIGS. 7A, 8B-F,9A-9B, and 15D-15F) is in the constrained configuration within the lumen 305 of the delivery catheter 304, the proximal retention feature 850 maintains engagement with the proximal portion 202 of the shunt 200, such that the shunt 200, shroud 800, and delivery catheter 304 translated together as a unit. As a result, proximal movement of the delivery catheter 304 transmits a corresponding proximal force to the shunt 200 and anchoring mechanism 229.

If the anchoring mechanism 229 is properly deployed within the CP angle cistern 138, proximal movement of the delivery catheter 304 will be resisted due abutment of the expanded anchoring mechanism 229 against an exterior surface of the arachnoid layer 115 and IPS wall 114. This resistance provides tactile feedback to the clinician indicating that the anchoring mechanism 229 is securely positioned within the CP angle cistern 138. The resistance may also provide visual feedback in the form of visual confirmation under fluoroscopy. For example, the force applied creates tension in the system that will visibly show separation between the distal end of the shroud 800 and the proximal end of the anchoring mechanism 229 due to the polymer construction of the shunt 200. Conversely, if little or no resistance is encountered upon proximal movement of the delivery catheter 304, and the delivery catheter 304 translates proximally without corresponding resistance, this may indicate that the anchoring mechanism 229 has not been fully deployed within the CP angle cistern 138 and/or that penetrating element 350 has not accessed CP angle cistern 138 and that anchor mechanism 229 deployed within IPS 102. In such cases, (1)(i) delivery wire 830 may be pulled proximally to withdraw the shunt 200 and shroud 800 into the delivery catheter lumen 305, (ii) the tether actuation mechanism 18 can be pushed down toward handle 10 of the delivery catheter 304 (i.e., in the opposite direction from arrow 18b in FIG. 14C) to actuate the guard member 400 distally to cover the penetrating element 350, and (iii) the delivery catheter 304 can be repositioned for another attempt to access CP angle cistern 138, or (2) the used delivery assembly 300 (i.e., delivery catheter 304, shroud 800, and shunt 200) can be withdrawn from the patient and the procedure can be reattempted with a new delivery assembly 300. Thus, the mechanical assessment using proximally movement of the delivery catheter 204 provides a relatively rapid intra-procedural check of deployment status, supplementing imaging-based evaluation and enabling confirmation of anchoring engagement without requiring additional imaging runs.

Lastly, the delivery catheter 304 is removed from the venous vasculature via venous access point of the patient (step 1130). For example, while pinning or holding delivery wire 830 in place, the delivery catheter 304 may be withdrawn proximally over the shroud 800 until the proximal retention feature 850 is no longer constrained within the lumen 305 of the delivery catheter 304, such that the shunt 200 may be released from the shroud 800. The delivery catheter 304, shroud 800, and delivery wire 830 may then be withdrawn from the venous vasculature of the patient.

In an alternative embodiment of the shunt deployment procedure 1000, a guidewire having a larger outer diameter and providing more support than the guidewire 902 may be used in place of the smaller guidewire 902 and the microcatheter 900. For example, the larger guide wire may have an outer diameter commensurate with the outer diameter of the microcatheter 900 (e.g., a 0.018” guide wire may be used). Thus, in steps 1010, 1020, 1030, 1040, and 1050, only the larger guide wire is used for accessing the IPS 102 and supporting navigation of delivery catheter 304 into the IPS 102 and distal to the targeted IPS segment 920.

In another alternative embodiment of the shunt deployment procedure 1000, instead of using a penetrating element 350 coupled to the delivery catheter 304, a stylet (not shown) slidably disposed within the lumen 304 of the delivery catheter 304 may be employed. In this case, after the distal portion 344 of the delivery catheter 304 has been navigated to the targeted segment 920 of the IPS 102 in step 1090, the stylet may be used to pierce through the IPS wall 114 and access the CP angle cistern 138, as well as to deploy the shunt 200 within the CP angle cistern 138. Details discussing the use of a stylet to access and deploy a shunt within a CP angle cistern are disclosed in PCT Publication WO2016/070147, which is expressly incorporated herein by reference.

It should be appreciated that, in contrast to anchor-based techniques (e.g., as illustrated in FIGS. 11-12), which primarily utilize a distal anchoring construct as a navigation aid to facilitate advancement and positioning of the delivery catheter 304 within the venous anatomy, the microcatheter-based approach illustrated in FIG. 22 provides an alternative navigation strategy without requiring deployment of an anchor within the IPS 102 that introduces additional procedural steps, including anchor delivery, deployment, and subsequent retrieval, as well as multiple device exchanges across the neurovascular anatomy. The microcatheter-based approach provides a predictable and controlled method of positioning the distal portion 344 of the delivery catheter 304 while reducing reliance on additional implanted support structures. The microcatheter-based approach can advantageously improve patient safety and procedural efficiency compared to anchor-based navigation techniques. Procedure time can be significantly reduced, for example on average by 50% or more, by eliminating anchor deployment and retrieval steps and reducing the number of device exchanges. In turn, fewer procedural steps may reduce reliance on fluoroscopic guidance, thereby lowering radiation exposure to the patient. Additionally, eliminating the need for anchor deployment within the dural venous sinuses, including the IPS 102 and CS 104 avoids radial expansion forces and mechanical interaction associated with anchor use, which may otherwise contribute to transient irritation or injury of adjacent cranial nerves. Accordingly, the microcatheter-based approach simplifies the procedure while maintaining controlled and reliable delivery of the shunt 200 into the SAS 116.

Although embodiments of the delivery catheter 304 have been disclosed for navigation and deployment of shunt 200, the delivery catheter 304 can also be used in other procedures where it is necessary to access and/or deploy devices into the SAS 116 of a patient. For example, embodiments of the delivery catheter 304 can be used to access the SAS 116 and deliver a therapeutic agent from the lumen 305 of the delivery catheter 304. Alternatively, a microcatheter can be navigated through the lumen 305 of the delivery catheter 304 to a location or region within the SAS 116, and a therapeutic agent can be delivered through the microcatheter into the SAS 116. Non-limiting examples of therapeutic agents that can be delivered to the SAS 116 with embodiments of delivery catheter 304 can include compositions comprising anti-sense RNA or anti-sense oligonucleotides, anti-bodies, anti-biotics, anti-vasospasm agents, biosimilars, chemotherapy agents, GABA receptor agonists, therapies intended for the treatment of neurodegenerative diseases (e.g., Alzheimer’s disease, Parkinson’s disease, Huntington’s disease, prion disease, motor neurone diseases, spinocerebellar ataxia, and spinal muscular atrophy), therapies intended for the treatment of trigeminal neuralgia, therapies intended for the treatment of pontine glioma, tissue plasminogen activator, and any other composition(s) intended to have a therapeutic effect on all or a portion(s) of the central nervous system.

While the delivery catheter navigation techniques described above enable controlled advancement of the delivery catheter 304 within the venous anatomy, accurate performance of the subsequent access to the IPS 102 and penetration through the IPS wall 114 into the SAS 116 remain dependent on fluoroscopic visualization of the IPS 102, the delivery catheter 304, and the penetrating element 350 (or alternatively a stylet) relative to the IPS wall 114. In particular, because the IPS 102 follows a complex three-dimensional and obliquely oriented course within the skull base, conventional fluoroscopic imaging techniques may not consistently provide a complete or anatomically faithful representation of the longitudinal extent of the IPS 102 or the spatial relationship between the penetrating element 350 and the IPS wall 114. As a result, during critical stages of the procedure—such as positioning the penetrating element 350 at the targeted IPS segment 920 and advancing the penetrating element 350 through the IPS wall 114 into the subarachnoid space 116—visualization limitations may reduce the operator’s ability to fully appreciate device orientation, depth of advancement, and penetration status within the IPS 102.

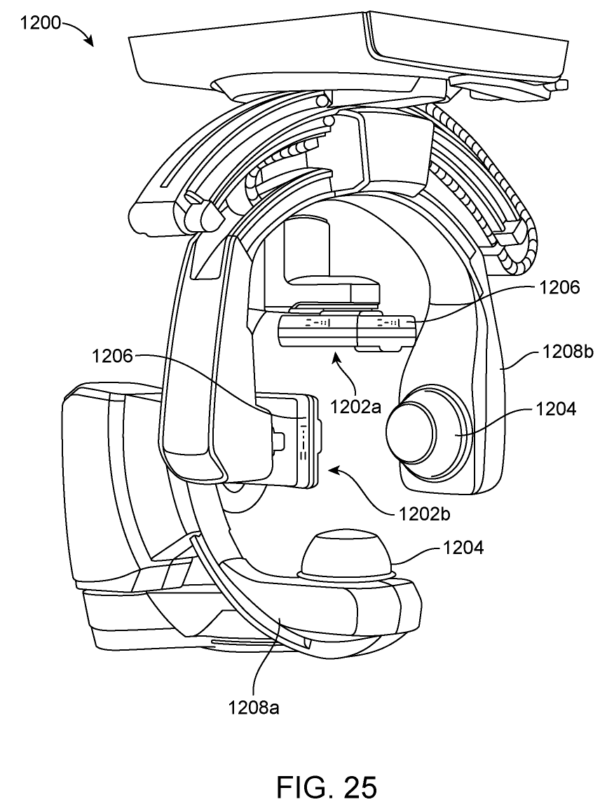

In one imaging technique, a bi-plane fluoroscopic imaging system may be utilized to visualize the relevant anatomy of the patient during any of the IPS navigation and SAS access procedures described above. For example, one embodiment of a bi-plane fluoroscopic imaging system 1200 is illustrated in FIG. 25. The fluoroscopic imaging system 1200 is configured for acquiring real-time fluoroscopic images of the patient simultaneously in two distinct projections of a region of interest, including the IPS 102 and surrounding anatomical structures, such as the SAS 116, during a medical procedure, e.g., one of the IPS navigation and SAS access procedures described above. To this end, the fluoroscopic imaging system 1200 comprises a first imaging assembly 1202a and a second imaging assembly 1202b arranged to acquire images from different orientations relative to the patient, thereby enabling simultaneous or near-simultaneous visualization of the IPS 102 from multiple perspectives.

Each imaging assembly 1202a, 1202b includes an x-ray source 1204 and a corresponding x-ray detector 1206 positioned on opposite sides of the patient (not shown) to define an imaging axis through the region of the interest in the patient. Each x-ray source 1204 may comprise an x-ray tube (not shown) configured for generating a collimated x-ray beam directed toward the corresponding x-ray detector 1206. Each x-ray detector 1206 may comprise a flat-panel detector or other digital imaging sensor configured for converting transmitted x-rays into image data representative of internal anatomical structures in the region of interest. Each x-ray detector 1206 may be operatively coupled to an image processing system (not shown) configured for reconstructing and displaying fluoroscopic images in real time during the IPS navigation and SAS access procedures.

Each imaging assembly 1202a, 1202b comprises a respective mechanical support structure, such as C-arms, that permits controlled positioning of the x-ray source 1204 and x-ray detector 1206 about the patient. In particular, in a biplane configuration, the imaging assembly 1202a comprises a frontal imaging C-arm 1208a configured for providing a frontal imaging view, such as an AP projection or an oblique projection relative to the patient, while the imaging assembly 1202b comprises a lateral imaging C-arm 1208b configured for providing a lateral imaging view oriented generally orthogonal to the frontal imaging plane. Each of the C-arms 1208a, 1208b may be independently adjustable to permit variation in rotational angulation about the patient as well as cranial and caudal tilt, thereby enabling acquisition of images at a plurality of projection angles tailored to the anatomy of the IPS 102.

The fluoroscopic imaging system 1200 may further comprise a control system (not shown) configured for coordinating operation of the x-ray sources 1204, x-ray detectors 1206, and the positioning mechanisms of the C-arms 1208a, 1208b. The control system may include one or more user input devices, such as joysticks, foot pedals, or graphical user interfaces, enabling a clinician to dynamically adjust projection angles and imaging parameters during the procedure. In some embodiments, the control system may further permit storage and recall of predetermined projection configurations associated with visualization of the IPS 102 or adjacent neurovascular structures.

The fluoroscopic imaging system 1200 may further comprise a display system configured for presenting fluoroscopic images to the clinician during the procedure. In a biplane configuration, images acquired from the first and second imaging assemblies 1202a, 1202b may be displayed simultaneously or in a synchronized manner to provide complementary views of the IPS 102 and associated instruments, including the delivery catheter 304 and penetrating element 350 (or alternatively a stylet). Such simultaneous visualization facilitates real-time assessment of device position, orientation, and advancement within the complex three-dimensional anatomy associated with the IPS 102.

Although a biplane fluoroscopic system is described herein, it will be appreciated that the imaging techniques disclosed may also be implemented using a single-plane fluoroscopic system. In such embodiments, a single imaging assembly may be repositioned between different projection angles to acquire multiple images sequentially. Accordingly, references to first and second imaging assemblies or projections are not intended to require simultaneous acquisition unless expressly stated.

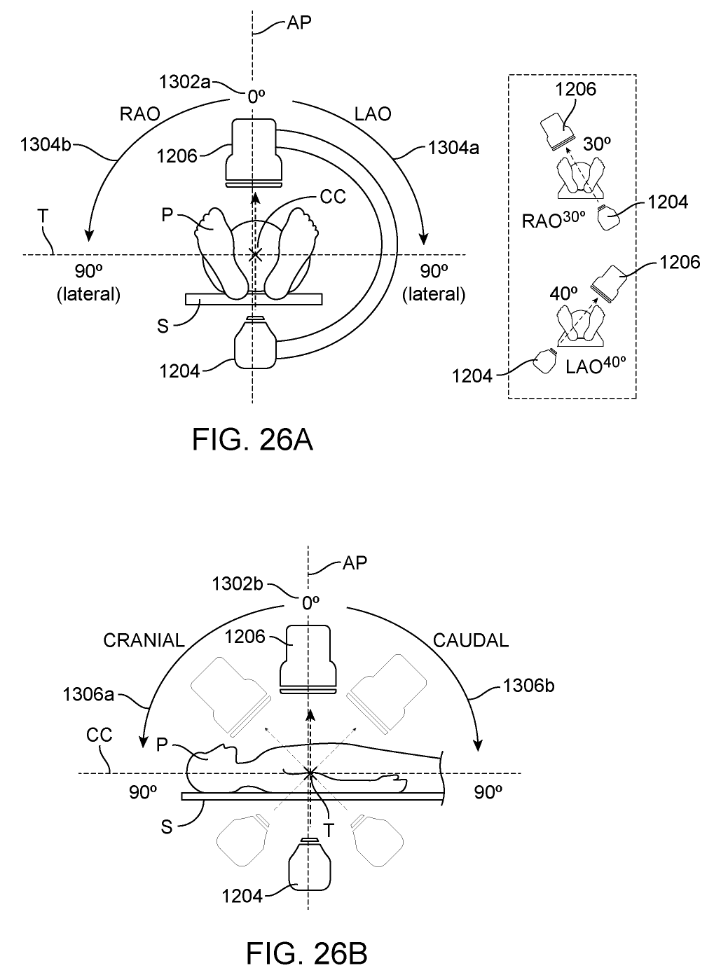

Referring now to FIGS. 26A and 26B, a coordinate framework for fluoroscopic imaging in which imaging projections are defined relative to an AP reference projection and a cranial-caudal axis of a patient P will now be described. The patient P is positioned in a supine orientation on a support surface 1300, defining a neural anatomical position. An AP reference projection is defined as an imaging axis oriented generally perpendicular to the support surface S and passing from an anterior side to a posterior side of the patient P. A cranial-caudal (CC) axis extends longitudinally through the patient P from the head toward the feet and serves as a common anatomical reference axis for angular positioning of either of the imaging assemblies 1202a, 1202b of the fluoroscopic imaging system 1200. A transverse (T) axis extends laterally across the patient P from a left side to a right side and is orthogonal to the cranial-caudal axis and the AP reference projection, thereby completing a three-dimensional anatomical reference framework.

As illustrated in FIG. 26A, the imaging geometry is defined relative to the AP reference projection, with angular adjustment occurring about the cranial-caudal axis. A neutral position 1302a at 0° corresponds to the AP reference projection, which is aligned perpendicular to the support surface 1300. From this neutral position 1302a, rotational angulation about the CC axis defines anterior oblique projections. Rotation in a counterclockwise direction 1304acorresponds to a right anterior oblique (RAO) projection, extending from the AP reference projection toward a lateral orientation of approximately 90 degrees. Rotation in a clockwise direction 1304b corresponds to a left anterior oblique (LAO) projection, likewise extending from the AP reference projection toward an opposite lateral orientation of approximately 90 degrees. The illustrated angular range from the AP reference projection to approximately ±90 degrees defines a continuum of rotationally oblique imaging geometries that may be selectively used to visualize anatomical structures, including the IPS 102. As one example, the combined x-ray source 1204 and x-ray detector 1206 may be rotated in the counterclockwise direction 1304ato a 30° RAO angle (shown in inset in FIG. 26A). As another example, the combined x-ray source 1204 and x-ray detector 1206 may be rotated in the clockwise direction 1304bto a 40° LAO angle (shown in inset in FIG. 26A).

As illustrated in FIG. 26B, the imaging geometry is defined by angular adjustment relative to the cranial-caudal axis of the patient P. A neutral position 1302b at 0° corresponds to an imaging axis aligned with the AP reference projection. From this neutral position 1302b, the imaging axis may be tilted about the transverse axis relative to the patient P to define cranial or caudal angulation. Tilting in a counterclockwise 1306a corresponds to cranial angulation, in which the imaging axis is directed toward the head of the patient. Tilting in a clockwise direction 1306b corresponds to caudal angulation, in which the imaging axis is directed toward the feet of the patient P. The illustrated range of motion extends from the neutral position 1302b to approximately 90 degrees in both cranial and caudal directions, defining a continuum of tilt-based imaging geometries that may be combined with rotational angulation to form compound oblique projections. The rotational angulation described with respect to FIG. 26A and the cranial–caudal angulation described with respect to FIG. 26B are defined within a common anatomical reference frame based on the AP reference projection and the cranial-caudal axis. These two degrees of freedom—rotation about the cranial-caudal axis and tilt about the transverse axis—may be combined to define compound oblique imaging projections, including oblique projections having both rotational and cranial or caudal components.

In one embodiment, the imaging geometries illustrated in FIGS. 26A and 26B are implemented using the fluoroscopic imaging system 1200. Each of the imaging assemblies 1202a, 1202bof the fluoroscopic imaging system 1200 is independently controllable and positionable relative to the patient P within a common anatomical reference frame defined by the AP reference projection, the cranial–caudal axis, and the transverse axis. Each of the imaging assemblies 1202a, 1202bis capable of both rotational angulation about the cranial–caudal axis and cranial or caudal tilt about the transverse axis. The imaging assemblies 1202a, 1202bmay be arranged at different nominal orientations relative to the patient P, such that the first imaging assembly 1202a is positioned to acquire images from a frontal or AP-referenced geometry, while the second imaging assembly 1202b is positioned to acquire images from a complementary geometry, such as a lateral or near-lateral orientation. From their respective nominal positions, each imaging assembly 1202a, 1202b may be independently adjusted using the rotational and cranial–caudal angulation degrees of freedom described with respect to FIGS. 26A and 26B to obtain selected imaging projections.

In particular, when an imaging assembly is positioned in a lateral or near-lateral orientation, rotational angulation may alternatively be described relative to a transverse or lateral anatomical reference axis, rather than the AP reference projection. In such configurations, rotational adjustments about the cranial-caudal axis define oblique projections relative to the lateral orientation of the imaging assembly, thereby providing an additional reference framework for describing compound oblique projections. In this manner, the first and second imaging assemblies 1202a, 1202b may be configured for acquiring different oblique projections of a region of interest, including compound oblique projections incorporating both rotational angulation and cranial or caudal tilt. The imaging assemblies 1202a, 1202b may be operated simultaneously in a biplane mode, although as described above, the imaging assembly of a single-plane fluoroscopic imaging system may be repositioned sequentially in a single-plane mode to obtain complementary views within the shared anatomical reference frame.

In some embodiments, the first imaging assembly 1202a is configured such that its neutral position corresponds to the AP reference projection, which is the same reference geometry represented by the neutral positions 1302a and 1302b in FIGS. 26A and 26B. Accordingly, in the neutral state of the first imaging assembly 1202a, the imaging axis is aligned with the AP reference projection and is oriented generally perpendicular to the support surface 1300, from which rotational angulation about the cranial-caudal axis and cranial–caudal tilt may be performed. In contrast, the second imaging assembly 1202b may be arranged such that its neutral position corresponds to a lateral reference projection, in which the imaging axis is oriented generally along the transverse axis of the patient P. In this neutral state, the imaging axis is approximately orthogonal to the AP reference projection, thereby providing a lateral or near-lateral baseline imaging geometry. From this neutral lateral orientation, the second imaging assembly 1202b may be adjusted using rotational angulation about the cranial–caudal axis and cranial or caudal tilt about the transverse anatomical axis to obtain selected imaging projections.

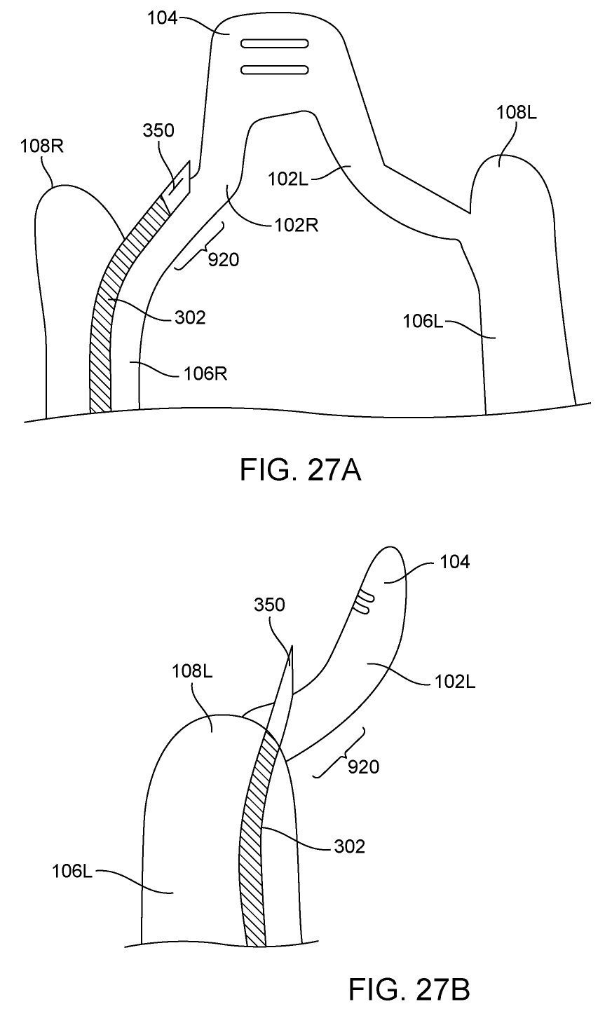

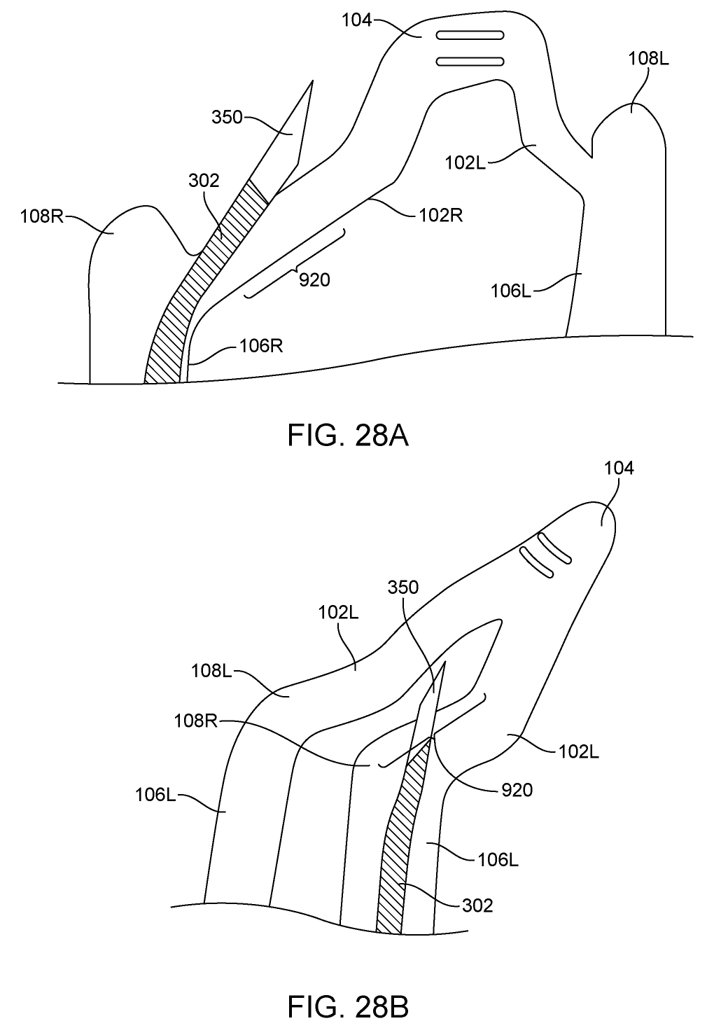

Referring now to FIGS. 27A-27B and FIGS.28A-28B, a fluoroscopic imaging technique that facilitates visualization during a medical procedure in which the delivery catheter 304 and associated penetrating element 350 are used to access an SAS 116 from an IPS 102 of a patient (see, e.g., FIGS. 22, 23A-23F and FIGS. 24A-24F) will be described. In one embodiment, the fluoroscopic imaging system 1200 illustrated in FIG. 25 is used to perform the fluoroscopic imaging technique.

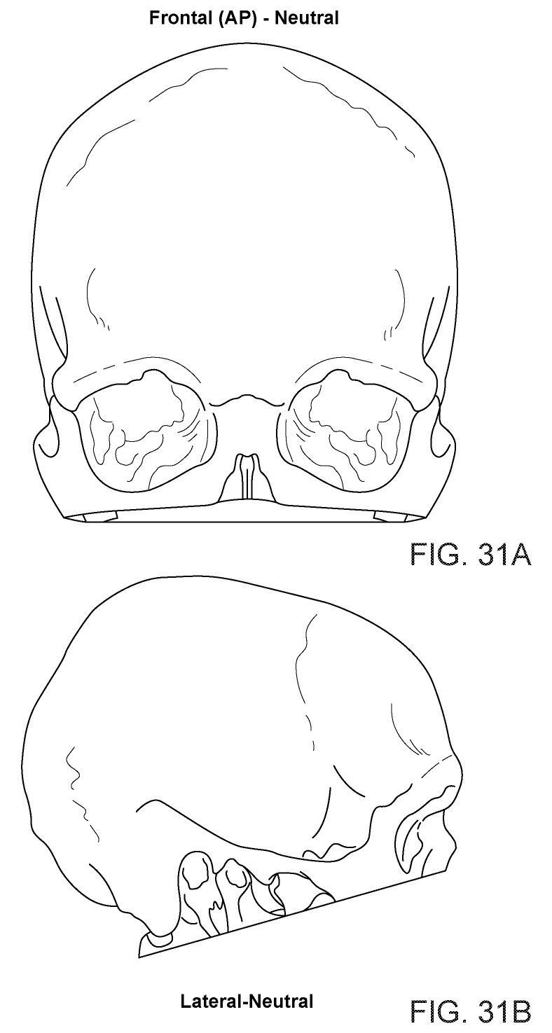

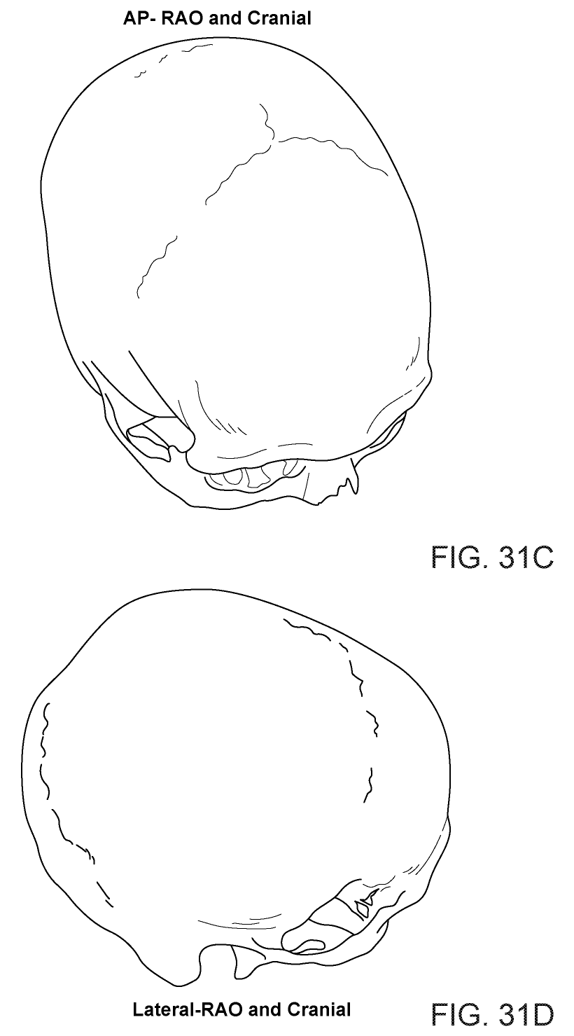

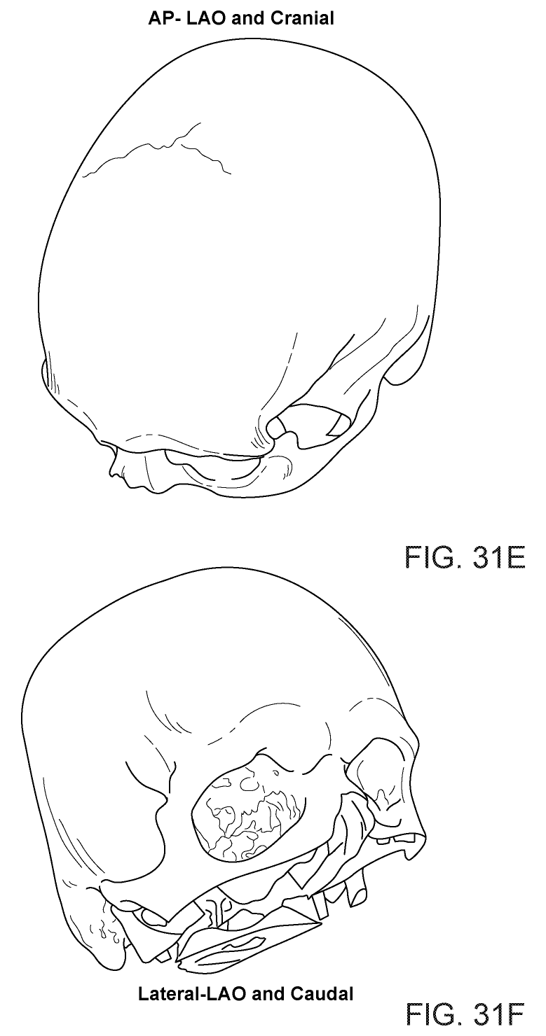

Referring first to FIGS. 27A-27B, the relevant anatomical structures within the base of the skull, including the IPS 102 (both left and right IPSs 102L, 102R), CS 104, jugular veins 106 (both left and right jugular veins 106L, 106R), and jugular bulbs 108 (both left and right jugular bulbs 108L, 108R) may be visualized using conventional projections, including an AP projection and a lateral projection. In the AP projection, the imaging axis is aligned with the AP reference projection (directed towards the front of the skull SK as illustrated in FIG. 31A), while in the lateral projection, the imaging axis is oriented generally orthogonal to the AP reference projection (directed towards the lateral side (in this case, the right lateral side) of the skull SK as illustrated in FIG. 31B).

Due to the oblique, three-dimensional course, of the IPS 102 relative to these standard projections, the segment 920 of the IPS 102 (in this case, the right IPS 102L) that is targeted for penetration into the CP angle cistern 138 may appear foreshortened in the AP projection (FIG. 27A), such that an apparent length of the targeted IPS segment 920 does not correspond to its anatomical length. This foreshortening may obscure portions of the longitudinal course of the IPS segment 920 and reduce the ability to determine a precise location along the IPS segment 920 for penetration of the wall of the IPS 102. In the lateral projection (FIG. 27B), although a complementary perspective is obtained relative to the AP projection, visualization of the targeted IPS segment may still be limited. In particular, because the lateral projection represents a single projection plane, spatial relationships of the delivery catheter 304 and penetrating element 350 relative to the targeted IPS segment 920 and adjacent structures may be ambiguous without additional imaging perspectives. Further, anatomical structures on the left and right sides of the patient, including the left and right IPSs 102, may be superimposed in the lateral projection, making it more difficult to distinguish between ipsilateral and contralateral anatomy and to isolate the targeted IPS segment 920. For example, as illustrated in FIG. 27B, the right IPS 102R, right jugular vein 106R, and right jugular bulb 108R may be hidden and obscured behind the left IPS 102L, left jugular vein 106L, and left jugular bulb 108L. As a result, the lateral projection alone may not reliably permit precise determination of a penetration location along the targeted IPS segment 920 or definitive confirmation that a penetrating element 350 has traversed the wall of the IPS 102 and entered the CP angle cistern 138.

Visualization of the relevant anatomical structures within the base of the skull, including the IPSs 102, CS 104, and jugular veins 106 may be greatly improved using certain oblique projections. In particular, as illustrated in FIG. 28A, the targeted IPS segment 920 (in this case, the targeted segment of the right IPS 102R) may be visualized in a first oblique projection including a rotational angulation relative to the AP reference projection and a cranial angulation relative to the cranial-caudal axis (e.g., an RAO/cranial projection or LAO/cranial projection), which is selected to reduce foreshortening of the targeted IPS segment 920, such that an apparent length of the targeted IPS segment 920 more closely corresponds to its anatomical length. As illustrated in FIG. 28B, the targeted IPS segment 920 may be visualized in a second oblique projection different from the first oblique projection, including a rotational angulation relative to a lateral or transverse axis and a cranial or caudal angulation relative to the cranial-caudal axis, which provides a complementary perspective that reduces superimposition of left and right IPSs 102 and facilitates improved visualization of the depth and trajectory of the penetrating element 350 relative to the targeted IPS segment 920 and the CP angle cistern 138.

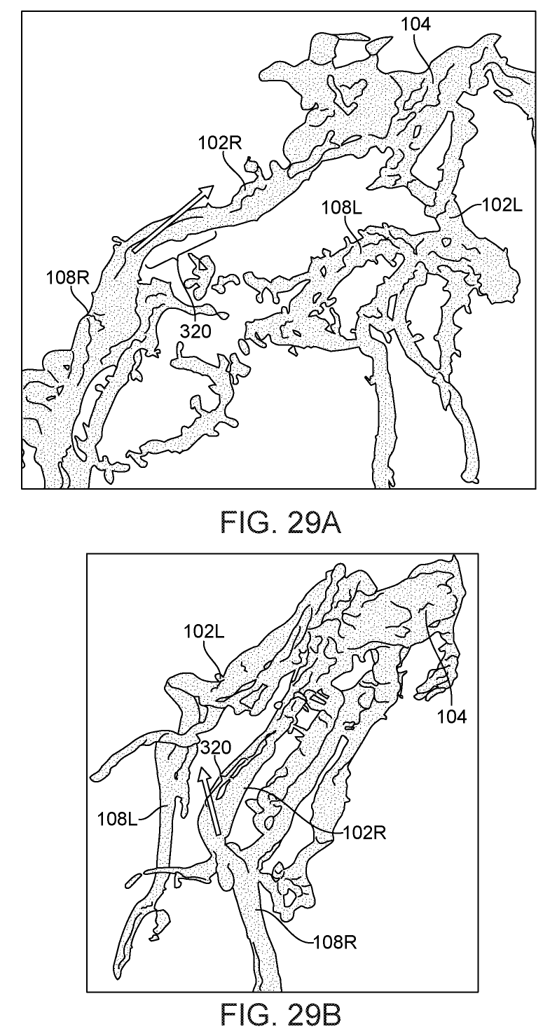

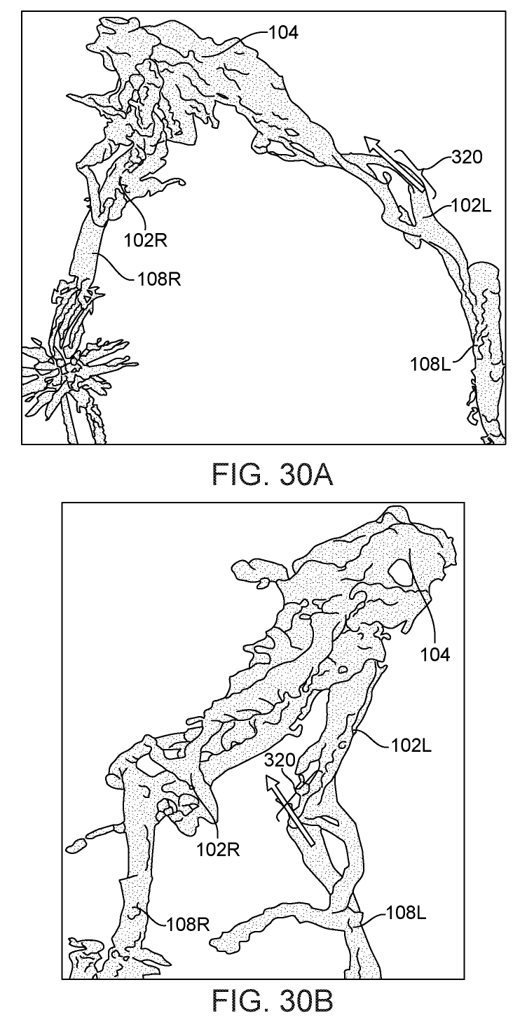

Referring now to FIGS. 29A and 30A, the first oblique projection may be obtained by positioning an imaging assembly AP reference orientation, corresponding to an imaging axis aligned with the AP reference projection, and further adjusting the imaging assembly from the AP reference projection using a combination of rotational angulation about the cranial–caudal axis and cranial angulation relative to the cranial–caudal axis. It has been discovered that selection of a rotational angulation within a range of approximately 25°–35° relative to the AP reference projection, and selection of a cranial angulation within a range of approximately 25°–35° relative to a neutral cranial–caudal orientation minimizes foreshortening of the targeted IPS segment 920. As illustrated, the first oblique projection may comprise different compound oblique configurations depending on laterality of the targeted IPS segment 920. For example, the first oblique projection may comprise either AP oblique RAO/cranial projection (FIG. 29A), such that the imaging axis is directed towards the skull, as illustrated in FIG. 31C, or an AP oblique LAO/cranial projection (FIG. 29B), such that the imaging axis is directed towards the skull, as illustrated in FIG. 31E. The AP oblique RAO/cranial projection may be used for visualization of a right IPS 102R, while the AP oblique LAO/cranial projection may be used for visualization of a left IPS 102L. In each case, the rotational angulation is directed toward the ipsilateral side of the targeted IPS segment 920 in combination with cranial angulation. In use, each of these oblique projections provides an imaging perspective in which a delivery catheter 304 and associated penetrating element 350 may be visualized along a substantial portion of the longitudinal course of the targeted IPS segment 920. In this orientation, the projection also provides meaningful visualization of the needle depth and trajectory (shown by arrow in FIGS. 29A and 30A) of the penetrating element 350 along the axis the targeted IPS segment 920, due to alignment of the imaging axis with the oblique anatomical course of the IPS 102. This facilitates positioning of the distal portion of the delivery catheter 104 relative to a selected penetration site and enables controlled alignment of the penetrating element 350 with respect to the targeted IPS segment 920.

Referring now to FIGS. 29B and 30B, the second oblique projection may be obtained by positioning an imaging assembly at a lateral or near-lateral reference orientation, corresponding to an imaging axis oriented generally along a transverse anatomical axis of the patient, and further adjusting the imaging axis using rotational angulation about the cranial–caudal axis and cranial or caudal angulation relative to the cranial–caudal axis. It has been discovered that selection of a rotational angulation within a range of approximately 25°–35° relative to the lateral axis, and a cranial or caudal angulation within a range of approximately 25°–35° relative to the cranial–caudal axis, provides sufficient isolation of the left and right IPSs 102 and improved visualization of the needle trajectory (shown by arrow in FIGS. 29B and 30B) in a plane complementary to the first oblique projection, including enhanced delineation of the exit location of the penetrating element 350 relative to the targeted IPS segment 920. As illustrated, the second oblique projection may comprise different compound oblique configurations depending on laterality of the targeted IPS segment 920. For example, the second oblique projection may comprise either lateral oblique RAO/cranial projection (FIG. 29B), such that the imaging axis is directed towards the skull, as illustrated in FIG. 31D, or a lateral oblique LAO/cranial projection (FIG. 30B), such that the imaging axis is directed towards the skull, as illustrated in FIG. 31F.

The lateral oblique RAO/cranial projection may be used for visualization of a right IPS 102, while the lateral oblique LAO/caudal projection may be used for visualization of a left IPS 102. In each case, the rotational component is defined relative to the lateral reference orientation, and the cranial or caudal tilt is selected to provide a complementary viewing angle relative to the first oblique projection. In use, these second oblique projections provide a complementary imaging perspective relative to the first oblique projection, in which the spatial relationship of the penetrating element 350 relative to the targeted IPS segment 920 and adjacent structures within the SAS 116 may be more clearly resolved in an orthogonal plane. In particular, this projection facilitates identification of the location at which the penetrating element 350 exits wall of the IPS 102 and enters the CP angle cistern 138, and enables improved assessment of whether the needle trajectory is directed appropriately within the CP angle cistern 138 rather than along or within adjacent venous structures.

During the medical procedure, the first and second oblique projections may be used in combination to guide advancement of the delivery catheter 304 and penetrating element 350 within the targeted IPS segment 920. The first oblique projection may be used to guide positioning of the delivery catheter 304 along the targeted IPS segment 920, to identify and maintain alignment with a selected penetration site, and to assess the depth and trajectory of the penetrating element 350 along the longitudinal axis of the targeted IPS segment 920. The second oblique projection may be used to monitor advancement of the penetrating element 350 in a complementary plane and to evaluate the location and orientation of needle exit through the wall of the IPS 102, including assessment of whether the penetrating element 350 has entered the CP angle cistern 138 at an intended anatomical location. By correlating the longitudinal and depth-related information from the first oblique projection with the orthogonal exit-location information from the second oblique projection, the clinician is provided with a more complete spatial understanding of needle position relative to the targeted IPS segment 920 and the CP angle cistern 138.

In one embodiment, the first and second oblique projections are acquired simultaneously using the biplane fluoroscopic imaging system 1200 illustrated in FIG. 25,. In this case, the first imaging assembly 1202a acquires the first oblique projection and a second imaging assembly 1202b acquires the second oblique projection. The imaging assemblies 1202a, 1202b may be independently positioned and maintained at the selected projection angles during manipulation of the delivery catheter 304 and penetrating element 350. In other embodiments, the first and second oblique projections may be acquired sequentially using a single-plane fluoroscopic imaging system by repositioning a single imaging assembly between the respective projection angles. The images obtained from the first and second oblique projections may be alternately visualized during the procedure to provide the complementary perspectives described herein.

Although particular embodiments have been shown and described herein, it will be understood by those skilled in the art that they are not intended to limit the present inventions, and it will be obvious to those skilled in the art that various changes, permutations, and modifications may be made (e.g., the dimensions of various parts, combinations of parts) without departing from the scope of the disclosed inventions, which is to be defined only by the following claims and their equivalents. The specification and drawings are, accordingly, to be regarded in an illustrative rather than restrictive sense. The various embodiments shown and described herein are intended to cover alternatives, modifications, and equivalents of the disclosed inventions, which may be included within the scope of the appended claims.

1. A method for deploying a shunt in a patient, the shunt comprising a lumen and a valve, the method comprising:
introducing a delivery catheter and one or more elongate navigation members into a venous access point in the patient, wherein the one or more elongate navigation members is disposed within a lumen of the delivery catheter;
navigating the delivery catheter and one or more elongate navigation members through a jugular vein and into a venous sinus of the patient;
distally advancing a distal portion of the delivery catheter distal to a targeted segment of the venous sinus;
removing the one or more elongate navigation members from the lumen of the delivery catheter;
introducing the shunt into the lumen of the delivery catheter;
distally advancing a shunt through the lumen of the delivery catheter within the venous sinus of the patient;
exposing a penetrating element coupled to a distal portion of the delivery catheter when the distal portion of the delivery catheter is distal to the targeted segment of the venous sinus;
proximally withdrawing the distal portion of the delivery catheter to the targeted segment of the venous sinus subsequent to exposing the penetrating element;
distally advancing the exposed penetrating element through a wall in the venous sinus at the targeted segment of the venous sinus to create an anastomosis between an intracranial subarachnoid space (SAS) and the venous sinus; and
implanting the shunt from the delivery catheter into the anastomosis.

2. The method of claim 1, wherein the shunt comprises a self-expanding distal anchoring mechanism, wherein implanting the shunt from the delivery catheter into the anastomosis comprises deploying the distal anchoring mechanism in the SAS.

3. The method of claim 2, further comprising:
after deploying the distal anchoring mechanism in the SAS, applying a proximal force to the delivery catheter while the shunt remains engaged with a proximal retention feature of a shroud disposed within the lumen of the delivery catheter; and
assessing sufficient deployment of the distal anchoring mechanism in the SAS based on a resistance to the proximal force applied to the delivery catheter.

4. The method of claim 1, wherein introducing the shunt into the lumen of the delivery catheter comprises:
connecting a transfer device to a handle of delivery catheter;
disposing a shroud assembly within a lumen of the transfer device, the should assembly comprising a shroud having a lumen in which the shunt is disposed and a delivery wire connected to the shroud; and
distally advancing the shroud assembly, while the shunt is disposed within the lumen of the shroud, from the lumen of the transfer device into the lumen of the delivery catheter.

5. The method of claim 4, wherein the shroud assembly further comprises a shroud retention feature that retains a proximal portion of the shunt when the shroud assembly is disposed within the lumen of the delivery catheter, the method further comprising unsheathing the shroud assembly from the lumen of the delivery catheter lumen, thereby releasing the proximal portion of the shunt from the retention feature.

6. The method of claim 4, further comprising flushing a lumen of the shunt and performing a valve patency confirmation test on the shunt before distally advancing the shunt assembly from the lumen of the transfer device into the lumen of the delivery catheter.

7. The method of claim 1, wherein the venous sinus is an inferior petrosal sinus (IPS), and wherein the anastomosis is created between the IPS and a cerebellopontine (CP) angle cistern of the SAS.

8. The method of claim 1, wherein the one or more elongate navigation members comprises a microcatheter and a guide wire disposed within a lumen of the microcatheter.

9. A method for fluoroscopic visualization during a medical procedure that accesses a subarachnoid space (SAS) from the inferior petrosal sinus (IPS) of a patient, comprising:
configuring a fluoroscopic imaging system to acquire images of the IPS during the medical procedure;
acquiring a first fluoroscopic image of the IPS in a first oblique projection including a rotational angulation in the range of 25°-35° relative to an anteroposterior (AP) reference projection and a cranial angulation relative to a cranial-caudal axis of the patient in the range of 25°-35°;
acquiring a second fluoroscopic image of the IPS in a second oblique projection different from the first oblique projection to provide a complementary view of the IPS; and
visualizing the first and second fluoroscopic image while performing the medical procedure.

10. The method of claim 9, wherein the first oblique projection comprises a right anterior (RAO)/cranial projection if the IPS is a right IPS and a left anterior (LAO)/cranial projection if the IPS is a left IPS.

11. The method of claim 9, wherein the first and second oblique projections are orthogonal to each other.

12. The method of claim 9, wherein the second oblique projection includes a rotational angulation in the range of 25°-35° relative to a lateral or transverse axis and a cranial or caudal angulation relative to the cranial-caudal axis of the patient in the range of 25°-35°.

13. The method of claim 9, wherein the fluoroscopic imaging system is a single-plane fluoroscopic imaging system, and the first and second fluoroscopic images are sequentially acquired.

14. The method of claim 9, wherein the fluoroscopic imaging system is a bi-plane fluoroscopic imaging system, and the first and second fluoroscopic images are simultaneously acquired.

15. The method of claim 14, wherein the biplane fluoroscopic imaging system comprises a frontal imaging arm and a lateral imaging arm, wherein the frontal imaging arm acquires the first fluoroscopic image and the lateral imaging arm acquires the second fluoroscopic image.

16. The method of claim 9, wherein visualizing the first and second fluoroscopic images while performing the medical procedure comprises guided advancement of a delivery catheter and a penetrating element associated with the delivery catheter.

17. The method of claim 16, wherein the guided advancement of the delivery catheter and penetrating element comprises determining a location of a penetration of a wall of the IPS along a longitudinal course of the IPS based on the first fluoroscopic image.

18. The method of claim 16, wherein the guided advancement of the delivery catheter and penetrating element comprises assessing whether the penetrating element has traversed a wall of the IPS and entered the SAS based on the first and second fluoroscopic images.

19. A method for fluoroscopic visualization during a medical procedure that accesses a subarachnoid space (SAS) from an inferior petrosal sinus (IPS) of a patient, comprising:
configuring a fluoroscopic imaging system to acquire images of the IPS during the medical procedure;
acquiring a first fluoroscopic image of the IPS in a first oblique projection configured for reducing foreshortening of a targeted segment of the IPS relative to a conventional anteroposterior (AP) projection; and
acquiring a second fluoroscopic image of the IPS in a second oblique projection different from the first oblique projection and configured to provide a complementary, at least partially orthogonal, view of the targeted segment of the IPS; and
visualizing the first and second fluoroscopic image while performing the medical procedure.

20. The method of claim 19,
wherein the first oblique projection enhances visualization of a delivery catheter and a penetrating element associated with the delivery catheter within a longitudinal course of the IPS and facilitating assessment of a trajectory of the penetrating element along the targeted segment of the IPS; and
wherein the second oblique projection facilitates confirmation that the penetrating element has traversed a wall of the IPS and identification of an exit location of the penetrating element in the targeted segment of the IPS.

21. The method of claim 19, wherein the first oblique projection comprises a right anterior (RAO)/cranial projection if the IPS is a right IPS and a left anterior (LAO)/cranial projection if the IPS is a left IPS.

22. The method of claim 19, wherein the first and second oblique projections are orthogonal to each other.

23. The method of claim 19, wherein the first oblique projection includes a rotational angulation in the range of 25°-35° relative to an anteroposterior (AP) reference projection and a cranial angulation relative to a cranial-caudal axis of the patient in the range of 25°-35°.

24. The method of claim 23, wherein the second oblique projection includes a rotational angulation in the range of 25°-35° relative to a lateral or transverse axis and a cranial or caudal angulation relative to the cranial-caudal axis of the patient in the range of 25°-35°.

25. The method of claim 19, wherein visualizing the first and second fluoroscopic image while performing the medical procedure comprises collectively using the first fluoroscopic image and the second fluoroscopic image to improve visualization of a longitudinal course of the IPS and a spatial relationship of the delivery catheter and penetrating element relative to the IPS and SAS.

26. The method of claim 19, wherein the fluoroscopic imaging system is a single-plane fluoroscopic imaging system, and the first and second fluoroscopic images are sequentially acquired.

27. The method of claim 19, wherein the fluoroscopic imaging system is a bi-plane fluoroscopic imaging system, and the first and second fluoroscopic images are simultaneously acquired.

28. The method of claim 27, wherein the biplane fluoroscopic imaging system comprises a frontal imaging arm and a lateral imaging arm, wherein the frontal imaging arm acquires the first fluoroscopic image and the lateral imaging arm acquires the second fluoroscopic image.

29. A method for deploying a shunt in a patient, the shunt comprising a lumen and a valve, the method comprising:
introducing a delivery catheter and one or more elongate navigation members into a venous access point in the patient, wherein the one or more elongate navigation members is disposed within a lumen of the delivery catheter;
navigating the delivery catheter and one or more elongate navigation members through a jugular vein and into a venous sinus of the patient;
navigating a distal portion of the delivery catheter within a targeted segment of the venous sinus of the patient;
removing the one or more elongate navigation members from the lumen of the delivery catheter;
introducing the shunt into the lumen of the delivery catheter;
distally advancing a shunt through the lumen of the delivery catheter within the venous sinus of the patient;
distally advancing a penetrating element coupled to a distal portion of the delivery catheter through a wall in the venous sinus at the targeted segment of the venous sinus to create an anastomosis between an intracranial subarachnoid space (SAS) and the venous sinus; and
implanting the shunt from the delivery catheter into the anastomosis.

30. The method of claim 29, wherein the shunt comprises a self-expanding distal anchoring mechanism, wherein implanting the shunt from the delivery catheter into the anastomosis comprises deploying the distal anchoring mechanism in the SAS.

31. The method of claim 30, further comprising:
after deploying the distal anchoring mechanism in the SAS, applying a proximal force to the delivery catheter while the shunt remains engaged with a proximal retention feature of a shroud disposed within the lumen of the delivery catheter; and
assessing sufficient deployment of the distal anchoring mechanism in the SAS based on a resistance to the proximal force applied to the delivery catheter.

32. The method of claim 29, wherein introducing the shunt into the lumen of the delivery catheter comprises:
connecting a transfer device to a handle of delivery catheter;
disposing a shroud assembly within a lumen of the transfer device, the should assembly comprising a shroud having a lumen in which the shunt is disposed and a delivery wire connected to the shroud; and
distally advancing the shroud assembly, while the shunt is disposed within the lumen of the shroud, from the lumen of the transfer device into the lumen of the delivery catheter.

33. The method of claim 32, wherein the shroud assembly further comprises a shroud retention feature that retains a proximal portion of the shunt when the shroud assembly is disposed within the lumen of the delivery catheter, the method further comprising unsheathing the shroud assembly from the lumen of the delivery catheter lumen, thereby releasing the proximal portion of the shunt from the retention feature.

34. The method of claim 32, further comprising flushing a lumen of the shunt and performing a valve patency confirmation test on the shunt before distally advancing the shunt assembly from the lumen of the transfer device into the lumen of the delivery catheter.

35. The method of claim 29, wherein the venous sinus is an inferior petrosal sinus (IPS), and wherein the anastomosis is created between the IPS and a cerebellopontine (CP) angle cistern of the SAS.

36. The method of claim 29, wherein the one or more elongate navigation members comprises a microcatheter and a guide wire disposed within a lumen of the microcatheter.
